SEQUENCE LISTING (1 sequence)

SEQ ID NO: 1 - DNA, 7904 bp, Human papillomavirus type 16
actacaataattcatgtataaaactaagggcgtaaccgaaatcggttgaaccgaaaccggttagtataaaagcagacattttatgcaccaaaagagaactgcaatgtttcaggacccacaggagcgacccagaaagttaccacagttatgcacagagctgcaaacaactatacatgatataatattagaatgtgtgtactgcaagcaacagttactgcgacgtgaggtatatgactttgcttttcgggatttatgtatagtatatagagatgggaatccatatgctgtatgtgataaatgtttaaagttttattctaaaattagtgagtatagacattattgttatagtgtgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaacacgtagagaaacccagctgtaatcatgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccataatctaccatggctgatcctgcaggtaccaatggggaagagggtacgggatgtaatggatggttttatgtagaggctgtagtggaaaaaaaaacaggggatgctatatcagatgacgagaacgaaaatgacagtgatacaggtgaagatttggtagattttatagtaaatgataatgattatttaacacaggcagaaacagagacagcacatgcgttgtttactgcacaggaagcaaaacaacatagagatgcagtacaggttctaaaacgaaagtatttgggtagtccacttagtgatattagtggatgtgtagacaataatattagtcctagattaaaagctatatgtatagaaaaacaaagtagagctgcaaaaaggagattatttgaaagcgaagacagcgggtatggcaatactgaagtggaaactcagcagatgttacaggtagaagggcgccatgagactgaaacaccatgtagtcagtatagtggtggaagtgggggtggttgcagtcagtacagtagtggaagtgggggagagggtgttagtgaaagacacactatatgccaaacaccacttacaaatattttaaatgtactaaaaactagtaatgcaaaggcagcaatgttagcaaaatttaaagagttatacggggtgagtttttcagaattagtaagaccatttaaaagtaataaatcaacgtgttgcgattggtgtattgctgcatttggacttacacccagtatagctgacagtataaaaacactattacaacaatattgtttatatttacacattcaaagtttagcatgttcatggggaatggttgtgttactattagtaagatataaatgtggaaaaaatagagaaacaattgaaaaattgctgtctaaactattatgtgtgtctccaatgtgtatgatgatagagcctccaaaattgcgtagtacagcagcagcattatattggtataaaacaggtatatcaaatattagtgaagtgtatggagacacgccagaatggatacaaagacaaacagtattacaacatagttttaatgattgtacatttgaattatcacagatggtacaatgggcctacgataatgacatagtagacgatagtgaaattgcatataaatatgcacaattggcagacactaatagtaatgcaagtgcctttctaaaaagtaattcacaggcaaaaattgtaaaggattgtgcaacaatgtgtagacattataaacgagcagaaaaaaaacaaatgagtatgagtcaatggataaaatatagatgtgatagggtagatgatggaggtgattggaagcaaattgttatgtttttaaggtatcaaggtgtagagtttatgtcatttttaactgcattaaaaagatttttgcaaggcatacctaaaaaaaattgcatattactatatggtgcagctaacacaggtaaatcattatttggtatgagtttaatgaaatttctgcaagggtctgtaatatgttttgtaaattctaaaagccatttttggttacaaccattagcagatgccaaaataggtatgttagatgatgctacagtgccctgttggaactacatagatgacaatttaagaaatgcattggatggaaatttagtttctatggatgtaaagcatagaccattggtacaactaaaatgccctccattattaattacatctaacattaatgctggtacagattctaggtggccttatttacataatagattggtggtgtttacatttcctaatgagtttccatttgacgaaaacggaaatccagtgtatgagcttaatgataagaactggaaatcctttttctcaaggacgtggtccagattaagtttgcacgaggacgaggacaaggaaaacgatggagactctttgccaacgtttaaatgtgtgtcaggacaaaatactaacacattatgaaaatgatagtacagacctacgtgaccatatagactattggaaacacatgcgcctagaatgtgctatttattacaaggccagagaaatgggatttaaacatattaaccaccaggtggtgccaacactggctgtatcaaagaataaagcattacaagcaattgaactgcaactaacgttagaaacaatatataactcacaatatagtaatgaaaagtggacattacaagacgttagccttgaagtgtatttaactgcaccaacaggatgtataaaaaaacatggatatacagtggaagtgcagtttgatggagacatatgcaatacaatgcattatacaaactggacacatatatatatttgtgaagaagcatcagtaactgtggtagagggtcaagttgactattatggtttatattatgttcatgaaggaatacgaacatattttgtgcagtttaaagatgatgcagaaaaatatagtaaaaataaagtatgggaagttcatgcgggtggtcaggtaatattatgtcctacatctgtgtttagcagcaacgaagtatcctctcctgaaattattaggcagcacttggccaaccactccgccgcgacccataccaaagccgtcgccttgggcaccgaagaaacacagacgactatccagcgaccaagatcagagccagacaccggaaacccctgccacaccactaagttgttgcacagagactcagtggacagtgctccaatcctcactgcatttaacagctcacacaaaggacggattaactgtaatagtaacactacacccatagtacatttaaaaggtgatgctaatactttaaaatgtttaagatatagatttaaaaagcattgtacattgtatactgcagtgtcgtctacatggcattggacaggacataatgtaaaacataaaagtgcaattgttacacttacatatgatagtgaatggcaacgtgaccaatttttgtctcaagttaaaataccaaaaactattacagtgtctactggatttatgtctatatgacaaatcttgatactgcatccacaacattactggcgtgctttttgctttgcttttgtgtgcttttgtgtgtctgcctattaatacgtccgctgcttttgtctgtgtctacatacacatcattaatactattggtattactattgtggataacagcagcctctgcgtttaggtgttttattgtatatattgtatttgtttatataccattatttttaatacatacacatgcacgctttttaattacataatgtatatgtacaaaatgtaattgttacatataattgttgtataccataacttactattttttcttttttattttcatatatattttttttttgtttgtttgtttgttttttaataaactgttatcacttaacaatgcgacacaaacgttctgcaaaacgcacaaaacgtgcatcggctacccaactttataaaacatgcaaacaggcaggtacatgtccacctgacattatacctaaggttgaaggcaaaactattgctgatcaaatattacaatatggaagtatgggtgtattttttggtgggttaggaattggaacagggtcgggtacaggcggacgcactgggtatattccattgggaacaaggcctcccacagctacagatacacttgctcctgtaagaccccctttaacagtagatcctgtgggcccttccgatccttctatagtttctttagtggaagaaactagttttattgatgctggtgcaccaacatctgtaccttccattcccccagatgtatcaggatttagtattactacttcaactgataccacacctgctatattagatattaataatactgttactactgttactacacataataatcccactttcactgacccatctgtattgcagcctccaacacctgcagaaactggagggcattttacactttcatcatccactattagtacacataattatgaagaaattcctatggatacatttattgttagcacaaaccctaacacagtaactagtagcacacccataccagggtctcgcccagtggcacgcctaggattatatagtcgcacaacacaacaagttaaagttgtagaccctgcttttataaccactcccactaaacttattacatatgataatcctgcatatgaaggtatagatgtggataatacattatatttttctagtaatgataatagtattaatatagctccagatcctgactttttggatatagttgctttacataggccagcattaacctctaggcgtactggcattaggtacagtagaattggtaataaacaaacactacgtactcgtagtggaaaatctataggtgctaaggtacattattattatgattttagtactattgattctgcagaagaaatagaattacaaactataacaccttctacatatactaccacttcacatgcagccttacctacttctattaataatggattatatgatatttatgcagatgactttattacagatacttctacaaccccggtaccatctgtaccctctacatctttatcaggttatattcctgcaaatacaacaattccttttggtggtgcatacaatattcctttagtatcaggtcctgatatacccattaatataactgaccaagctccttcattaattcctatagttccagggtctccacaatatacaattattgctgatgcaggtgacttttatttacatcctagttattacatgttacgaaaacgacgtaaacgtttaccatattttttttcagatgtctctttggctgcctagtgaggccactgtctacttgcctcctgtcccagtatctaaggttgtaagcacggatgaatatgttgcacgcacaaacatatattatcatgcaggaacatccagactacttgcagttggacatccctattttcctattaaaaaacctaacaataacaaaatattagttcctaaagtatcaggattacaatacagggtatttagaatacatttacctgaccccaataagtttggttttcctgacacctcattttataatccagatacacagcggctggtttgggcctgtgtaggtgttgaggtaggccgtggtcagccattaggtgtgggcattagtggccatcctttattaaataaattggatgacacagaaaatgctagtgcttatgcagcaaatgcaggtgtggataatagagaatgtatatctatggattacaaacaaacacaattgtgtttaattggttgcaaaccacctataggggaacactggggcaaaggatccccatgtaccaatgttgcagtaaatccaggtgattgtccaccattagagttaataaacacagttattcaggatggtgatatggttgatactggctttggtgctatggactttactacattacaggctaacaaaagtgaagttccactggatatttgtacatctatttgcaaatatccagattatattaaaatggtgtcagaaccatatggcgacagcttatttttttatttacgaagggaacaaatgtttgttagacatttatttaatagggctggtgctgttggtgaaaatgtaccagacgatttatacattaaaggctctgggtctactgcaaatttagccagttcaaattattttcctacacctagtggttctatggttacctctgatgcccaaatattcaataaaccttattggttacaacgagcacagggccacaataatggcatttgttggggtaaccaactatttgttactgttgttgatactacacgcagtacaaatatgtcattatgtgctgccatatctacttcagaaactacatataaaaatactaactttaaggagtacctacgacatggggaggaatatgatttacagtttatttttcaactgtgcaaaataaccttaactgcagacgttatgacatacatacattctatgaattccactattttggaggactggaattttggtctacaacctcccccaggaggcacactagaagatacttataggtttgtaacatcccaggcaattgcttgtcaaaaacatacacctccagcacctaaagaagatccccttaaaaaatacactttttgggaagtaaatttaaaggaaaagttttctgcagacctagatcagtttcctttaggacgcaaatttttactacaagcaggattgaaggccaaaccaaaatttacattaggaaaacgaaaagctacacccaccacctcatctacctctacaactgctaaacgcaaaaaacgtaagctgtaagtattgtatgtatgttgaattagtgttgtttgttgtttatatgtttgtatgtgcttgtatgtgcttgtaaatattaagttgtatgtgtgtttgtatgtatggtataataaacacgtgtgtatgtgtttttaaatgcttgtgtaactattgtgtcatgcaacataaataaacttattgtttcaacacctactaattgtgttgtggttattcattgtatataaactatatttgctacatcctgtttttgttttatatatactaaattttgtagcgccagcggccattttgtagcttcaaccgaattcggttgcatgctttttggcacaaaatgtgtttttttaaatagttctatgtcagcaactatagtttaaacttgtacgtttcctgcttgccatgcgtgccaaatccctgttttcctgacctgcactgcttgccaaccattccattgttttttacactgcactatgtgcaactactgaatcactatgtacattgtgtcatataaaataaatcactatgcgccaacgccttacataccgctgttaggcacatatttttggcttgttttaactaacctaattgcatatttggcataaggtttaaacttctaaggccaactaaatgtcaccctagttcatacatgaactgtgtaaaggttagtcatacattgttcatttgtaaaactgcacatgggtgtgtgcaaaccgttttgggttacacatttacaagcaacttatataataatactaa

(19) United States
(12) Patent Application Publication
(10) Pub. No.: US 2013/0137086 A1
(43) Pub. Date: May 30, 2013

(54) HPV DNA Methylation Patterns of Diagnostic or Prognostic Significance in Cervical Cancer Screening

(71) Applicants: Janet L. Brandsma, Guilford, CT (US); Paul M. Lizardi, Wallingford, CT (US)

(72) Inventors: Janet L. Brandsma, Guilford, CT (US); Paul M. Lizardi, Wallingford, CT (US)

(73) Assignee: YALE UNIVERSITY, New Haven, CT (US)

(21) Appl. No.: 13/696,546

(22) PCT Filed: May 9, 2011
(86) PCT No.: PCT/US11/35803

§ 371 Date: Nov. 6, 2012

Related U.S. Application Data
(60) Provisional application No. 61/332,376, filed on May 7, 2010.

(51) Int. Cl. C12Q 1/70

(52) U.S. Cl. CPC C12Q 1/708
USPC 435/5

(57) ABSTRACT

Disclosed are methods, compositions, devices, and systems for assessing cancer potential, state, stage, risk of progression, prognosis, etc. of a subject based on determining the methylation state of human papillomavirus (HPV) in a sample from the subject. The cancers assessed generally can be cancer associated with or caused by HPV. For example, cervical cancer, vulvar cancer, penile cancer, anal cancer, and head and neck cancer can be associated with HPV. It has been discovered that certain patterns, profiles, and sets of methylation of HPV genomes are correlated with different cancer potential, state, stage, risk of progression, prognosis, etc.

### CROSS-REFERENCE TO RELATED APPLICATIONS

This application claims benefit of U.S. Provisional Application No. 61/332,376, filed May 7, 2010. Application No. 61/332,376, filed May 7, 2010, is hereby incorporated herein by reference in its entirety.

### FIELD OF THE INVENTION

The disclosed invention is generally in the field of assessment of cancer risk and specifically in the area of assessment of cervical cancer risk.

### BACKGROUND

More than 50% of vulvar, vaginal and anal cancers and virtually 100% of cervical cancers arise from lesions caused by human papillomavirus (HPV) infections (zur Hausen, 1996). Although a vaccine that prevents genital HPV infection is now available, it covers only two types of cancer-associated HPV, and it is not therapeutic so women who have already acquired HPV will continue to develop anogenital cancers. The most prevalent and preventable HPV-associated cancer is cervical carcinoma. Since the introduction of public health screening programs in the 1940's (Pap smear) screening, the incidence of cervical cancer has dropped dramatically in the U.S and other developed countries. Still in countries that have not implemented screening programs, cervical cancer remains the number one cancer of women.

The malignant progression of cervical cells involves deregulation of HPV genes as well as cellular genes. As a lesion progresses from CIN-1 to carcinoma, the viral genome undergoes physical and transcriptional alterations. Normally it is maintained as a multicopy extrachromosomal episome, but during malignant progression it frequently integrates into cellular DNA. The frequency of HPV16 DNA integration is nil in CIN-1 lesions, 6% in CIN-2 lesions, 19% in CIN-3 lesions and 55% in cervical cancers (Vinokurova, 2008). The HPV transcriptional program is tightly linked to the differentiation state of the host epithelial cell. In the basal cell layer of CIN-1 lesions, the early genes E1, E2, E6 and E7 are barely transcribed, but they are dramatically and selectively upregulated in the spinous layer. In the granular layer early gene expression subsides and is replaced by high level expression of the late genes L1 and L2 that encode the viral capsid proteins, which are assembled into new virions in terminally differentiated cells. As CIN-1 progresses to CIN-2/3, cellular differentiation diminishes and the late genes are no longer expressed. Moreover, as CIN-2/3 lesions progress to carcinoma, the E6 and E7 oncogenes become overexpressed. Because the E2 protein regulates the E6/E7 promoter, loss of the E2 gene would deregulate E6/E7 expression. In fact, the E2 gene is frequently lost during integration of HPV DNA into the cellular genome. This mechanism cannot, however, be universal because many cervical cancers contain only episomal HPV DNA. Other mechanisms controlling viral gene expression are largely unknown.

A major mechanism regulating gene expression is DNA methylation. DNA methylation is an epigenetic process that is non-mutational, heritable and reversible. It is critical for normal development and for cellular differentiation, including epithelial differentiation (Paradisi, 2008). The basic mechanism of DNA methylation has been known since the 1980s, but intensive efforts to understand the role of DNA methylation in gene regulation have been more recent. The best studied aspect of DNA methylation concerns gene promoters. Many but not all studies indicate that the absence of DNA methylation allows full expression of a gene, while DNA methylation turns a gene off. These studies are only a start though, and the vast majority of CpGs occur outside promoters.

Cervical cancer screening is based on the cytologic evaluation of Papanicolau-stained cells (Pap smear). Cytology detects low grade and high grade squamous intraepithelial lesions, LSIL and HSIL, respectively. Patients with LSIL generally do not require treatment because most LSILs regress spontaneously. On the other hand, patients with HSIL are immediately referred to colposcopy for biopsy. The histologic manifestations of HPV infection are cervical intraepithelial neoplasia (CIN). Women with low grade CIN (CIN-1) are monitored more frequently but usually do not require treatment. In contrast, women with high grade lesions (CIN-2/3) are scheduled for surgical excision or laser ablation. Despite its success, the Pap test is not an ideal screening tool because it is subjective. In addition, the sensitivity of a single Pap test for detecting a high grade lesion is only about 50% (Spitzer, 2002), which is why the test is typically repeated annually. The Pap test also has a low specificity, i.e. about 20% of test results falsely show a high grade lesion (Spitzer, 2002). Consequently, many women are referred to colposcopy for biopsy on the basis of the Pap test result, only to find out later that the histology, the definitive diagnosis, shows no high grade lesion. Finally, more than 5% of cervical samples cannot be definitely diagnosed by cytology—those that contain atypical squamous cells of undetermined significance (ASCUS). ASCUS samples are routinely tested for high-risk HPV DNA and, if positive, the patient is referred for colposcopic follow-up. The frequency of Pap testing, the large number of histologic follow-ups, and the cost of treating all women with CIN-2/3 makes cervical cancer prevention an expensive proposition.

It would be useful to have a screening test that provides better and/or alternative assessment of the risk of cervical dysplasia and progression to cancer. For example, such a screening test could reduce the number of false positives, mis-assessments, and/or unnecessary follow-up and invasive testing. As another example, such a screening test could indicate whether women with a cytologic assessment of low or moderate risk nevertheless could benefit from further testing. The disclosed methods solve these and more problems and provide these desired goals.

### BRIEF SUMMARY

Disclosed are methods, compositions, devices, and systems for assessing cancer potential, state, stage, risk of progression, prognosis, etc. of a subject based on determining the methylation state of human papillomavirus (HPV) in a sample from the subject. The cancers assessed generally can be cancer associated with or caused by HPV. For example, cervical cancer, vulvar cancer, penile cancer, anal cancer, and head and neck cancer can be associated with HPV. It has been discovered that certain patterns, profiles, and sets of methylation of HPV genomes are correlated with different cancer potential, state, stage, risk of progression, prognosis, etc.

Disclosed are methods comprising determining the methylation state of a set of CpG dinucleotides in HPV sequences in a sample of a subject and identifying the cancer potential state of the subject based on the methylation state of the set of 3 or more CpG dinucleotides. The set can comprise a set of 3 or more of the CpG dinucleotides that correspond to positions 3887, 3937, 3941, 4016, 4084, 4238, 4247, 4259, 4268, 4275, 4425, 4435, 4439, 4537, 4892, 4904, 4922, 5126, 5171, 5177, 5376, 5600, 5606, 5609, 5615, 5707, 5724, 5925, 5961, 6365, 6387, 6455, 6579, 6648, 6729, 6794, 7032, and 7089 of HPV16. The HPV sequences in the sample can be, for example, HPV16 sequences or HPV18 sequences.

In some forms, a methylation site percentage for the set of 3 or more CpG dinucleotides of over 37.0% indicates that the subject should be tested further. In some forms, a methylation site percentage for the set of 3 or more CpG dinucleotides of up to 37.0% indicates that the subject need not be tested further for at least 6 months.

In some forms, the set of 3 or more CpG dinucleotides can comprise the CpG dinucleotide corresponding to position 5177 of HPV16. In some forms, the set of 3 or more CpG dinucleotides can further comprise the CpG dinucleotide corresponding to position 6455 of HPV16. In some forms, the set of 3 or more CpG dinucleotides can further comprise the CpG dinucleotides corresponding to positions 3887, 3937, 3941, 5126, 5171, 5600, 5609, 5707, 5724, 5925, and 6365 of HPV16. In some forms, the set of 3 or more CpG dinucleotides can further comprise the CpG dinucleotides corresponding to positions 5615 and 5961 of HPV16. In some forms, the set of 3 or more CpG dinucleotides can further comprise the CpG dinucleotides corresponding to positions 5126, 5171, 5600, 5609, 5707, 5724, 5925, and 6365 of HPV16. In some forms, the set of 3 or more CpG dinucleotides can further comprise the CpG dinucleotides corresponding to positions 3887, 3937, and 3941 of HPV16. In some forms, the set of 3 or more CpG dinucleotides can further comprise the CpG dinucleotides corresponding to positions 5376, 5606, 6387, and 6579 of HPV16. In some forms, the set of 3 or more CpG dinucleotides can further comprise the CpG dinucleotides corresponding to positions 5126, 5171, 5600, 5609, 5707, 5724, 5925, and 6365 of HPV16. In some forms, the set 3 or more CpG dinucleotides can comprise the CpG dinucleotides that correspond to positions 3887, 3937, 3941, 4439, 5126, 5600, 5707, 5724, and 6387 of HPV16. In some forms, the set of 3 or more CpG dinucleotides can further comprise the CpG dinucleotides corresponding to positions 4435 and 6729 of HPV16. In some forms, the set of 3 or more CpG dinucleotides for which the methylation state is determined can comprise the 113 CpG dinucleotides of HPV16.

In some forms, a methylation site percentage for the set of 3 or more CpG dinucleotides of below 20.0% indicates a low cancer potential state, a methylation site percentage for the set of 3 or more CpG dinucleotides from 20.0% to 50.0% indicates a moderate cancer potential state, and a methylation site percentage for the set of 3 or more CpG dinucleotides of over 50.0% indicates a high cancer potential state.

In some forms, identification of the cancer potential state of the subject can be further based on both cytologic screening and on the methylation state of the set of CpG dinucleotides. In some forms, a cytology assessment of low-grade squamous intraepithelial lesion (LSIL) and a methylation site percentage for the set of 3 or more CpG dinucleotides of below 20.0% indicates a low cancer potential state, a methylation site percentage for the set of 3 or more CpG dinucleotides from 20.0% to 50.0% indicates a moderate cancer potential state, and a methylation site percentage for the set of 3 or more CpG dinucleotides of over 50.0% indicates a high cancer potential state.

In some forms, a cytology assessment of atypical squamous cells (ASC) and a methylation site percentage for the set of 3 or more CpG dinucleotides of below 15.0% indicates a low cancer potential state, a cytology assessment of ASC and a methylation site percentage for the set of 3 or more CpG dinucleotides from 15.0% to 35.0% indicates a moderate cancer potential state, and a cytology assessment of ASC and a methylation site percentage for the set of 3 or more CpG dinucleotides of over 35.0% indicates a high cancer potential state.

In some forms, a cytology assessment of high-grade squamous intraepithelial lesion (HSIL) and a methylation site percentage for the set of 3 or more CpG dinucleotides of below 10.0% indicates a low cancer potential state, a cytology assessment of HSIL and a methylation site percentage for the set of 3 or more CpG dinucleotides from 10.0% to 35.0% indicates a moderate cancer potential state, and a cytology assessment of HSIL and a methylation site percentage for the set of 3 or more CpG dinucleotides of over 35.0% indicates a high cancer potential state.

In some forms, a methylation site percentage for the set of 3 or more CpG dinucleotides of over 37.0% indicates that the subject should be tested further. In some forms, a methylation site percentage for the set of 3 or more CpG dinucleotides of over 37.0% indicates that the subject should be referred for colposcopy. In some forms, a methylation site percentage for the set of 3 or more CpG dinucleotides of up to 37.0% indicates that the subject need not be tested further for at least 6 months.

In some forms, identification of the cancer potential state of the subject can be further based on both histologic screening and on the methylation state of the set of CpG dinucleotides. In some forms, a histology assessment of HPV-associated intraepithelial neoplasia grade 1 and a methylation site percentage for the set of 3 or more CpG dinucleotides of below 20.0% indicates a low cancer potential state, a methylation site percentage for the set of 3 or more CpG dinucleotides from 20.0% to 50.0% indicates a moderate cancer potential state, and a methylation site percentage for the set of 3 or more CpG dinucleotides of over 50.0% indicates a high cancer potential state.

In some forms, a histology assessment of HPV-associated intraepithelial neoplasia grade 2 and a methylation site percentage for the set of 3 or more CpG dinucleotides of below 15.0% indicates a low cancer potential state, a histology assessment of HPV-associated intraepithelial neoplasia grade 2 and a methylation site percentage for the set of 3 or more CpG dinucleotides from 15.0% to 50.0% indicates a moderate cancer potential state, and a histology assessment of HPV-associated intraepithelial neoplasia grade 2 and a methylation site percentage for the set of 3 or more CpG dinucleotides over 35.0% indicates a high cancer potential state.

In some forms, a histology assessment of HPV-associated intraepithelial neoplasia grade 3 and a methylation site percentage for the set of 3 or more CpG dinucleotides of below 35.0% indicates a moderate cancer potential state, and a histology assessment of HPV-associated intraepithelial neoplasia grade 3 and a methylation site percentage for the set of 3 or more CpG dinucleotides over 35.0% indicates a high cancer potential state.

In some forms, identification of the cancer potential state of the subject can be further based on cytologic screening, on histologic screening, and on the methylation state of the set of CpG dinucleotides. In some forms, a cytology assessment of LSIL, a histology assessment of HPV-associated intraepithelial neoplasia grade 1, and a methylation site percentage for the set of 3 or more CpG dinucleotides of below 20.0% indicates a low cancer potential state, a methylation site percentage for the set of 3 or more CpG dinucleotides from 20.0% to 50.0% indicates a moderate cancer potential state, and a methylation site percentage for the set of 3 or more CpG dinucleotides of over 50.0% indicates a high cancer potential state.

In some forms, a cytology assessment of LSIL, a histology assessment of HPV-associated intraepithelial neoplasia grade 2, and a methylation site percentage for the set of 3 or more CpG dinucleotides of below 15.0% indicates a low cancer potential state, a cytology assessment of LSIL, a histology assessment of HPV-associated intraepithelial neoplasia grade 2, and a methylation site percentage for the set of 3 or more CpG dinucleotides from 15.0% to 50.0% indicates a moderate cancer potential state, and a cytology assessment of LSIL, a histology assessment of HPV-associated intraepithelial neoplasia grade 2, and a methylation site percentage for the set of 3 or more CpG dinucleotides of over 50.0% indicates a high cancer potential state.

In some forms, a cytology assessment of LSIL, a histology assessment of HPV-associated intraepithelial neoplasia grade 3, and a methylation site percentage for the set of 3 or more CpG dinucleotides of below 10.0% indicates a low cancer potential state, a cytology assessment of LSIL, a histology assessment of HPV-associated intraepithelial neoplasia grade 3, and a methylation site percentage for the set of 3 or more CpG dinucleotides from 10.0% to 35.0% indicates a moderate cancer potential state, and a cytology assessment of LSIL, a histology assessment of HPV-associated intraepithelial neoplasia grade 3, and a methylation site percentage for the set of 3 or more CpG dinucleotides of over 35.0% indicates a high cancer potential state.

In some forms, a cytology assessment of ASC, a histology assessment of HPV-associated intraepithelial neoplasia grade 1, and a methylation site percentage for the set of 3 or more CpG dinucleotides of below 15.0% indicates a low cancer potential state, a cytology assessment of ASC, a histology assessment of HPV-associated intraepithelial neoplasia grade 1, and a methylation site percentage for the set of 3 or more CpG dinucleotides from 15.0% to 50.0% indicates a moderate cancer potential state, and a cytology assessment of ASC, a histology assessment of HPV-associated intraepithelial neoplasia grade 1, and a methylation site percentage for the set of 3 or more CpG dinucleotides of over 50.0% indicates a high cancer potential state.

In some forms, a cytology assessment of ASC, a histology assessment of HPV-associated intraepithelial neoplasia grade 2, and a methylation site percentage for the set of 3 or more CpG dinucleotides of below 15.0% indicates a low cancer potential state, a cytology assessment of ASC, a histology assessment of HPV-associated intraepithelial neoplasia grade 2, and a methylation site percentage for the set of 3 or more CpG dinucleotides from 15.0% to 35.0% indicates a moderate cancer potential state, and a cytology assessment of ASC, a histology assessment of HPV-associated intraepithelial neoplasia grade 2, and a methylation site percentage for the set of 3 or more CpG dinucleotides of over 35.0% indicates a high cancer potential state.

In some forms, a cytology assessment of ASC, a histology assessment of HPV-associated intraepithelial neoplasia grade 3, and a methylation site percentage for the set of 3 or more CpG dinucleotides of below 35.0% indicates a moderate cancer potential state, and a cytology assessment of ASC, a histology assessment of HPV-associated intraepithelial neoplasia grade 3, and a methylation site percentage for the set of 3 or more CpG dinucleotides of over 35.0% indicates a high cancer potential state.

In some forms, a cytology assessment of HSIL, a histology assessment of HPV-associated intraepithelial neoplasia grade 1, and a methylation site percentage for the set of 3 or more CpG dinucleotides of below 15.0% indicates a low cancer potential state, a cytology assessment of HSIL, a histology assessment of HPV-associated intraepithelial neoplasia grade 1, and a methylation site percentage for the set of 3 or more CpG dinucleotides from 15.0% to 35.0% indicates a moderate cancer potential state, and a cytology assessment of HSIL, a histology assessment of HPV-associated intraepithelial neoplasia grade 1, and a methylation site percentage for the set of 3 or more CpG dinucleotides of over 35.0% indicates a high cancer potential state,

In some forms, a cytology assessment of HSIL, a histology assessment of HPV-associated intraepithelial neoplasia grade 2, and a methylation site percentage for the set of 3 or more CpG dinucleotides of below 10.0% indicates a low cancer potential state, a cytology assessment of HSIL, a histology assessment of HPV-associated intraepithelial neoplasia grade 2, and a methylation site percentage for the set of 3 or more CpG dinucleotides from 10.0% to 35.0% indicates a moderate cancer potential state, and a cytology assessment of HSIL, a histology assessment of HPV-associated intraepithelial neoplasia grade 2, and a methylation site percentage for the set of 3 or more CpG dinucleotides of over 35.0% indicates a high cancer potential state,

In some forms, a cytology assessment of HSIL, a histology assessment of HPV-associated intraepithelial neoplasia grade 3, and a methylation site percentage for the set of 3 or more CpG dinucleotides of below 20.0% indicates a moderate cancer potential state, and a cytology assessment of HSIL, a histology assessment of HPV-associated intraepithelial neoplasia grade 3, and a methylation site percentage for the set of 3 or more CpG dinucleotides of over 20.0% indicates a high cancer potential state.

In some forms, identification of the cancer potential state of the subject can be further based on both histologic screening and on the methylation state of the set of CpG dinucleotides. In some forms, a histology assessment of CIN-1 and a methylation site percentage for the set of 3 or more CpG dinucleotides of below 20.0% indicates a low cancer potential state, a methylation site percentage for the set of 3 or more CpG dinucleotides from 20.0% to 50.0% indicates a moderate cancer potential state, and a methylation site percentage for the set of 3 or more CpG dinucleotides of over 50.0% indicates a high cancer potential state.

In some forms, a histology assessment of CIN-2 and a methylation site percentage for the set of 3 or more CpG dinucleotides of below 15.0% indicates a low cancer potential state, a histology assessment of CIN-2 and a methylation site percentage for the set of 3 or more CpG dinucleotides from 15.0% to 50.0% indicates a moderate cancer potential state, and a histology assessment of CIN-2 and a methylation site percentage for the set of 3 or more CpG dinucleotides over 35.0% indicates a high cancer potential state.

In some forms, a histology assessment of CIN-3 and a methylation site percentage for the set of 3 or more CpG dinucleotides of below 35.0% indicates a moderate cancer potential state, and a histology assessment of CIN-3 and a methylation site percentage for the set of 3 or more CpG dinucleotides over 35.0% indicates a high cancer potential state.

In some forms, identification of the cancer potential state of the subject can be further based on cytologic screening, on histologic screening, and on the methylation state of the set of CpG dinucleotides. In some forms, a cytology assessment of LSIL, a histology assessment of CIN-1, and a methylation site percentage for the set of 3 or more CpG dinucleotides of below 20.0% indicates a low cancer potential state, a methylation site percentage for the set of 3 or more CpG dinucleotides from 20.0% to 50.0% indicates a moderate cancer potential state, and a methylation site percentage for the set of 3 or more CpG dinucleotides of over 50.0% indicates a high cancer potential state.

In some forms, a cytology assessment of LSIL, a histology assessment of CIN-2, and a methylation site percentage for the set of 3 or more CpG dinucleotides of below 15.0% indicates a low cancer potential state, a cytology assessment of LSIL, a histology assessment of CIN-2, and a methylation site percentage for the set of 3 or more CpG dinucleotides from 15.0% to 50.0% indicates a moderate cancer potential state, and a cytology assessment of LSIL, a histology assessment of CIN-2, and a methylation site percentage for the set of 3 or more CpG dinucleotides of over 50.0% indicates a high cancer potential state.

In some forms, a cytology assessment of LSIL, a histology assessment of CIN-3, and a methylation site percentage for the set of 3 or more CpG dinucleotides of below 10.0% indicates a low cancer potential state, a cytology assessment of LSIL, a histology assessment of CIN-3, and a methylation site percentage for the set of 3 or more CpG dinucleotides from 10.0% to 35.0% indicates a moderate cancer potential state, and a cytology assessment of LSIL, a histology assessment of CIN-3, and a methylation site percentage for the set of 3 or more CpG dinucleotides of over 35.0% indicates a high cancer potential state.

In some forms, a cytology assessment of ASC, a histology assessment of CIN-1, and a methylation site percentage for the set of 3 or more CpG dinucleotides of below 15.0% indicates a low cancer potential state, a cytology assessment of ASC, a histology assessment of CIN-1, and a methylation site percentage for the set of 3 or more CpG dinucleotides from 15.0% to 50.0% indicates a moderate cancer potential state, and a cytology assessment of ASC, a histology assessment of CIN-1, and a methylation site percentage for the set of 3 or more CpG dinucleotides of over 50.0% indicates a high cancer potential state.

In some forms, a cytology assessment of ASC, a histology assessment of CIN-2, and a methylation site percentage for the set of 3 or more CpG dinucleotides of below 15.0% indicates a low cancer potential state, a cytology assessment of ASC, a histology assessment of CIN-2, and a methylation site percentage for the set of 3 or more CpG dinucleotides from 15.0% to 35.0% indicates a moderate cancer potential state, and a cytology assessment of ASC, a histology assessment of CIN-2, and a methylation site percentage for the set of 3 or more CpG dinucleotides of over 35.0% indicates a high cancer potential state.

In some forms, a cytology assessment of ASC, a histology assessment of CIN-3, and a methylation site percentage for the set of 3 or more CpG dinucleotides of below 35.0% indicates a moderate cancer potential state, and a cytology assessment of ASC, a histology assessment of CIN-3, and a methylation site percentage for the set of 3 or more CpG dinucleotides of over 35.0% indicates a high cancer potential state.

In some forms, a cytology assessment of HSIL, a histology assessment of CIN-1, and a methylation site percentage for the set of 3 or more CpG dinucleotides of below 15.0% indicates a low cancer potential state, a cytology assessment of HSIL, a histology assessment of CIN-1, and a methylation site percentage for the set of 3 or more CpG dinucleotides from 15.0% to 35.0% indicates a moderate cancer potential state, and a cytology assessment of HSIL, a histology assessment of CIN-1, and a methylation site percentage for the set of 3 or more CpG dinucleotides of over 35.0% indicates a high cancer potential state,

In some forms, a cytology assessment of HSIL, a histology assessment of CIN-2, and a methylation site percentage for the set of 3 or more CpG dinucleotides of below 10.0% indicates a low cancer potential state, a cytology assessment of HSIL, a histology assessment of CIN-2, and a methylation site percentage for the set of 3 or more CpG dinucleotides from 10.0% to 35.0% indicates a moderate cancer potential state, and a cytology assessment of HSIL, a histology assessment of CIN-2, and a methylation site percentage for the set of 3 or more CpG dinucleotides of over 35.0% indicates a high cancer potential state,

In some forms, a cytology assessment of HSIL, a histology assessment of CIN-3, and a methylation site percentage for the set of 3 or more CpG dinucleotides of below 20.0% indicates a moderate cancer potential state, and a cytology assessment of HSIL, a histology assessment of CIN-3, and a methylation site percentage for the set of 3 or more CpG dinucleotides of over 20.0% indicates a high cancer potential state.

In some forms, the identified cancer potential state can indicate that follow-up and/or treatment that would otherwise be indicated for a given cytology assessment, histology assessment, or both need not be performed. For example, for lesions with a low cancer potential state, the test result could reduce the unnecessary treatment of CIN lesions. Care providers of patients with low-grade CIN-1 lesions with a low cancer potential state would have objective evidence to discourage them from unnecessarily treating CIN-1. CIN-1 is often overtreated. For CIN-2 (and eventually CIN-3) lesions with a low cancer potential state, therapeutic intervention could be safely delayed, which would enable spontaneous regression in many cases. In some forms, the identified cancer potential state can indicate that follow-up and/or treatment that otherwise would not be indicated for a given cytology assessment, histology assessment, or both should be performed.

In some forms, the method can further comprise performing a histologic screen of the subject for cervical cancer potential at an interval based on the identified cancer potential state. In some forms, the method can further comprise creating a record of the identification of the cancer potential state of the subject. In some forms, the method can further comprise testing the subject for cervical cancer potential at an interval based on the identified cancer potential state. In some forms, the method can further comprise testing the subject for cervical cancer potential in a mode or manner based on the identified cancer potential state. In some forms, the method can further comprise treating the subject at an interval based on the identified cancer potential state. In some forms, the method can further comprise treating the subject in a mode or manner based on the identified cancer potential state.

In some forms, the method can further comprise a second determination of the methylation state of a set of CpG dinucleotides in HPV sequences in a sample of the subject, wherein a change or no change in the prognosis of the subject. In some forms, an increase in methylation, a change in methylation pattern (to a more negative pattern), or both indicates a worsening prognosis of the subject. In some forms, a decrease in methylation, a change in methylation pattern (to a more positive pattern), or both indicates an improving prognosis of the subject. In some forms, no significant change in methylation indicates no change in prognosis of the subject. The second determination can be done at any suitable interval of time, for example, 6 months, 12 months, etc. Additional and multiple determinations can be made over time for the same subject. The change in methylation in second and subsequent determinations can be used, for example, to determine what further tests to perform, if any, and at what intervals. For example, a change in methylation to a more negative pattern can indicate that the subject should be referred for colposcopy. As another example, the frequency of cytological re-testing can be determined by the trend of methylation.

In some forms, the determination of methylation state can be performed following a therapeutic intervention, such as chemotherapy, surgery, etc. If the subject has residual HPV infection, the methylation state can be used to determine the frequency and aggressiveness of follow-up treatment and/or testing. For example, a subject with residual infection and a high cancer potential state could benefit from more frequent monitoring. If the HPV DNA methylation analysis showed a low cancer potential state, the lesion might have a good prognosis, e.g., might clear the infection without further intervention.

In some forms, the cancer potential state for HPV-associated adenocarcinoma can be identified by determining the methylation state of a set of CpG dinucleotides in HPV16 sequences in a sample of a subject. For example, precursor states of HPV-associated adenocarcinoma can be identified and assessed based on the methylation state of sets of CpG dinucleotides.

Also disclosed are methods comprising determining the methylation state of a set of CpG dinucleotides in HPV16 sequences in a sample of a subject and identifying the cancer potential state of the subject based on the methylation state of the set of CpG dinucleotides, where the set comprises the CpG dinucleotides that correspond to positions 3887, 3937, 3941, 4439, 5126, 5600, 5707, 5724, and 6387 of HPV16. In some forms, the set of CpG dinucleotides can further comprise the CpG dinucleotides corresponding to positions 4435 and 6729 of HPV16.

Also disclosed are methods comprising determining the methylation state of a set of CpG dinucleotides in HPV16 sequences in a sample of a subject and identifying the cancer potential state of the subject based on the pattern of methylation of the set of CpG dinucleotides, where a pattern of methylation corresponding to methylation pattern A indicates a low cancer potential state, and where a pattern of methylation corresponding to methylation pattern C indicates a high cancer potential state.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows the distribution among cervical samples of methylated and unmethylated CpGs in the HPV16 open reading frames and long control region. The diamonds represent CpGs that were unmethylated in all samples (⋄), in a single sample (grey diamond), or in multiple samples (♦). The number of CpGs in each ORF/LCR is listed to the right of each schematic.

FIG. 2 shows HPV16 DNA methylation profiles of the individual cervical samples. The HPV16 DNA methylation profile of each sample at each potential methylation site is shown. The numbers inside each plot are the sample numbers and the adjacent letters are the HPV16 DNA methylation pattern (A, B or C). Nucleotides other than the 113 CpGs are not shown. The organization of the HPV16 genome with sequential numbering of the CpGs is shown at the bottom of the figure.

FIG. 3 shows a heat map of the relationship of the thirteen HPV16 DNA methylation profiles. The heat map was derived by Cluster analysis and, above it, the corresponding dendrogram with the sample numbers vertically oriented and in black and P-values horizontally oriented and in grey. The original values are shown without scaling. Each branch is labeled with the corresponding HPV16 methylation pattern (A, B or C). The CpGs are shown in the same order as they occur in the HPV16 genome; every fourth CpG is listed on the y-axis. The reference to cytosine 4239 actually refers to the cytosine at position 4238 in the HPV16 genome. Not shown are the 52 CpGs without any methylation and the eight with partially missing data (see Table 4).

FIGS. 4A, 4B, and 4C show HPV16 DNA methylation patterns A, B and C. The panels show three HPV16 methylation patterns: A, B and C, respectively. The x-axis is the same for all panels, but only the CpGs with methylation in each pattern are labeled. The nucleotide numbers are those of the W12 isolate of HPV16 (Flores et al., 1999) (Genbank AF125673; SEQ ID NO:1). The bars represent CpGs that were methylated in only one sample per pattern (□); only two samples (patterns A and B) or two to four samples (pattern C) (grey), and all five samples of pattern C (▪). One sample in pattern A was missing data at position 1509, and one in pattern C, at position 4247. Not shown are the 52 unmethylated CpGs. The y-axis shows the mean percentage of HPV16 genomes per sample with methylation at each CpG plus the S.E.M. The reference to cytosines 4239 and 4424 actually refer to the cytosines at positions 4238 and 4425 in the HPV16 genome.

FIG. 5 shows the position of each CpG (diamond) in the context of the full-length gene or LCR. The numbers designate the start and end of an open reading frame or LCR.

FIGS. 6A and 6B show the cases with low frequency (A) and high frequency (B) methylation in the first study. Amplicons #11, 15, 16 and 17 were analyzed in the 2nd and 3rd studies.

FIGS. 7A and 7B show the cases with no or low frequency methylation (A) and high frequency methylation (B). The shading indicates cases in the first (▪), second (▪) or third (▪) study.

FIG. 8 shows methylation percentage of genomes methylated at the indicated CpG positions. The dots indicate the percentage of HPV16 genomes with methylation at each CpG, determined by averaging multiple DNA sequence reads per CpG. The pathologic diagnosis of each case is shown on the left. ICC refers to invasive cervical carcinoma.

### DETAILED DESCRIPTION

The disclosed method and compositions may be understood more readily by reference to the following detailed description of particular embodiments and the Example included therein and to the Figures and their previous and following description.

More than 50% of vulvar, vaginal and anal cancers and virtually 100% of cervical cancers arise from lesions caused by human papillomavirus (HPV) infections (zur Hausen, 1996). Cervical cancer is caused by high-risk types of human papillomavirus (HPV). HPV DNA testing is highly sensitive, and a negative test effectively rules out cervical pre/cancer. The clinical utility of HPV DNA testing is however severely limited by the non-specifically of a positive test, due to the high prevalence of innocuous HPV in the general population.

Routine cervical cytology screening (Pap testing) has greatly reduced the incidence of cervical squamous cell carcinoma (SCC) but has had minimal impact on adenocarcinoma (ADC). Yet ADC accounts for up to 25% of all cervical cancer and its incidence is rising, especially among younger women. ADC also has a worse prognosis than SCC. The Pap test is further limited by high false negative and false-positive rates. Its efficacy therefore depends on the regular rescreening of all women with (potentially false) negative tests. It also depends on the clinical follow-up of over four million women with cytologic abnormalities each year. The problem is that despite extensive follow-up, fewer than 5% of such women are diagnosed with cervical precancer and just 12,000 with invasive cervical cancer.

It has been discovered, by mapping of all 113 methylation sites in the HPV16 genomes contained in patient samples of cervical cells, that cervical samples exhibit one of three distinct DNA methylation patterns and that these methylation patterns are highly correlated with the cervical diagnoses arrived at by pathologic examination and consonant with what is known regarding the biology of HPV-associated malignant progression. It was also discovered that a specific HPV16 DNA methylation pattern distinguishes high-grade CIN-2/3 vs. low-grade CIN-1 or cytologically negative infection. It has also been discovered that a specific set of methylation sites in the HPV genome are strongly correlated to the stage and severity of dysplasia in cervical cells, thus allowing the more accurate identification of patients that would benefit from, for example, further testing, biopsy, histologic assessment, monitoring, and/or treatment. Although the relationship of HPV methylation and cervical cancer stage and development has been examined in the past, the present discovery solves the problem of providing a more accurate assessment (based on the location of methylation sites assessed) while requiring measurement at only a limited set of methylation sites (based on the significance of the correlation of these sites to the stage and severity of dysplasia in cervical cells.

In some forms, the disclosed methods also solve the problem identifying those patients with low risk cytologic assessments that nevertheless would benefit from, for example, further testing, biopsy, histologic assessment, monitoring, and/or treatment. Currently, it is recommended that patients with low risk cytologic assessments (from a Pap test, for example) be retested on a standard schedule based mostly on age. However, some patients may develop cervical dysplasia or progress sooner than average. Thus, new tests that can indicate patients that may benefit from more frequent retesting or further testing will be useful. The disclosed methods solve this problem by providing assessment of a specific set of methylation sites in the HPV genome that can indicate that the patient is at greater risk of progression in dysplasia of cervical cells and of progression to cervical cancer. For example, the disclosed methods can identify patients that might have been mis-assessed in cytologic testing; indicating that these patients are at greater risk than their cytologic assessment indicates.

In some forms, the disclosed methods also solve the problem identifying those patients moderate risk cytologic assessments that likely would not benefit from, for example, further testing, biopsy, histologic assessment, monitoring, and/or treatment. Currently, it is recommended that patients with moderate risk cytologic assessments (from a Pap test, for example) be examined by colposcopy and cervical biopsy. However, these more invasive procedures are, in hindsight, only useful for a fraction of these patients. Thus, new tests that can reduce the number of unnecessary or premature further testing will be useful. The disclosed methods solve this problem by providing assessment of a specific set of methylation sites in the HPV genome that can indicate that the patient is at greater risk of progression in dysplasia of cervical cells and of progression to cervical cancer. The disclosed methods can be used to identify those patients more likely to benefit from, for example, further testing, biopsy, histologic assessment, monitoring, and/or treatment. For example, the disclosed methods can identify patients that might have been mis-assessed in cytologic testing; indicating that these patients are at greater risk than their cytologic assessment indicates.

In some forms, the disclosed methods also solve the problem identifying those patients with high risk cytologic assessments and/or high risk histologic assessments that would benefit from, for example, more aggressive further testing, biopsy, histologic assessment, monitoring, and/or treatment. Although patients given a high risk assessment in cytologic screening and histologic screening are currently given further assessment, it can be useful to know if a patient has other indicators of cancer. Thus, new tests that can indicate patients that may benefit from more aggressive testing and treatment will be useful. The disclosed methods solve this problem by providing assessment of a specific set of methylation sites in the HPV genome that can indicate and/or confirm that the patient is at greater risk of and/or from cervical cancer.

Molecular diagnostics are the wave of the future. A commercial test for high-risk HPV DNA is FDA-approved, but it is not useful for cervical cancer screening because HPV DNA positivity occurs not only in high-grade lesions, but also in low grade lesions and subclinical infections, which are far more prevalent. Molecular biomarkers of malignant progression are actively being sought for cervical cancer, but so far none has been adequate for clinical use. A hallmark of carcinogenesis is deregulation of cellular gene expression. Deregulation causes the overexpression of genes that promote cellular proliferation and the silencing of other genes that protect cells against tumor formation. The malignant progression of cervical cells involves deregulation of HPV genes as well as cellular genes.

To better understand the role of DNA methylation in carcinogenesis, there is great desire to compare normal vs. malignant cells for differences in their DNA methylation patterns. Unfortunately, bisulfite-sequencing, the most accurate method for mapping the methylation status of each CpG in a DNA sequence, is not adequate for this endeavor due to the very large size of the human genome (Bernstein, 2007). In contrast, the method is entirely sufficient for mapping the relatively tiny genomes of viruses. Viruses cause an estimated 15% of all human cancers (zur Hausen, 1991), and their study over the past thirty years has been integral to our current understanding of molecular processes that regulate both normal and transformed cells (DiMaio, 2006).

A new generation of DNA tests based on CpG methylation patterns promises tremendous improvements in the prevention of human cancer. Tests for colorectal cancer screening are likely to become the first such tests. Disclosed herein are DNA methylation tests for improved cervical cancer screening. No new sample collection procedure is required for clinical application of the disclosed methods because cervical cells are already collected in a suitable medium (for use in Pap testing, for example). The clinical and economic benefits of the disclosed methods should be dramatic given the magnitude of the limitations of the current cervical cancer screening paradigm.

The disclosed methods analyze HPV DNA but differ from current HPV tests because the methods analyze the DNA for specific patterns of methylation. This critical additional (or substitute) step solves the problem of the non-specificity of HPV DNA testing alone. The fact that HPV DNA testing (without methylation analysis) is already approved for use in cervical cancer screening indicates clinical applicability of the discoveries described herein.

A key requirement for the development of a DNA methylation based test is the discovery of specific CpGs (sites of potential methylation) that are methylated in a given cancer and unmethylated in negative cells from the same tissue. Such sites have been discovered in the genome of HPV16, which causes half of all cervical cancers. The frequency of methylation at these sites increases from 0% in negative specimens to 83% in cervical cancers. The methylation frequency correlates with the risk of malignant progression as assessed by cervical pathology, but it measures different biologic properties because individual lesions with the same histologic diagnosis have different DNA methylation patterns. Certain DNA methylation patterns are disclosed herein to have diagnostic and/or prognostic utility. The discovered HPV16 DNA methylation patterns are diagnostic and/or prognostic of the risk of malignant progression. In different forms, the disclosed methods can (i) expedite the detection of significant cervical lesions, so that they can be treated promptly, (ii) greatly reduce the large number of innocuous cervical lesions currently receiving intensive but unnecessary clinical follow-up, and (iii) greatly reduce the high annual cost of cervical cancer prevention, estimated at $5 billion.

It was also discovered that specific patterns of HPV DNA methylation correlate with the neoplastic state of HPV-infected cervical cells. The HPV16 methylomes in 13 specimens of cervical cells were mapped by bisulfite-sequencing (Brandsma et al., 2009). The results revealed three distinct HPV16 DNA methylation patterns and a variant of one pattern. Significant concordance between the patterns and the pathologic diagnoses was also found. Finally, the most distinctive pattern—HPV16 DNA methylation pattern C—correlated with high-grade cervical intraepithelial neoplasia (CIN-2 and CIN-3), the lesions that are the most important lesions to detect, since their excision prevents cervical cancer.

One particularly interesting case was a low-grade CIN-1 lesion with a variant of pattern C designated C2. The major feature of C2, in addition to methylation at the pattern C-specific CpGs, was heavy methylation in the viral enhancer upstream of the E6/E7 promoter, suggesting that E6/E7 expression may have been compromised. Additional analyses showed that this lesion had the lowest viral load of all 13 cases. Moreover, it spontaneously regressed within one year. As HPV persistence requires continued E6/E7 expression, it was realized that high level methylation of the viral enhancer is a defensive host response that mechanistically mediates the regression of cervical lesions. Thus, the C2 pattern can be used to indicate a chance of regression.

Disclosed are methods, compositions, devices, and systems for assessing cancer potential, state, stage, risk of progression, prognosis, etc. of a subject based on determining the methylation state of human papillomavirus (HPV) in a sample from the subject. The cancers assessed generally can be cancer associated with or caused by HPV. For example, cervical cancer, vulvar cancer, penile cancer, anal cancer, and head and neck cancer can be associated with HPV. It has been discovered that certain patterns, profiles, and sets of methylation of HPV genomes are correlated with different cancer potential, state, stage, risk of progression, prognosis, etc.

Disclosed are methods comprising determining the methylation state of a set of CpG dinucleotides in HPV sequences in a sample of a subject and identifying the cancer potential state of the subject based on the methylation state of the set of 3 or more CpG dinucleotides. The set can comprise a set of 3 or more of the CpG dinucleotides that correspond to positions 3887, 3937, 3941, 4016, 4084, 4238, 4247, 4259, 4268, 4275, 4425, 4435, 4439, 4537, 4892, 4904, 4922, 5126, 5171, 5177, 5376, 5600, 5606, 5609, 5615, 5707, 5724, 5925, 5961, 6365, 6387, 6455, 6579, 6648, 6729, 6794, 7032, and 7089 of HPV16. The HPV sequences in the sample can be, for example, HPV16 sequences or HPV18 sequences.

In some forms, a methylation site percentage for the set of 3 or more CpG dinucleotides of over 37.0% indicates that the subject should be tested further. In some forms, a methylation site percentage for the set of 3 or more CpG dinucleotides of up to 37.0% indicates that the subject need not be tested further for at least 6 months.

In some forms, a methylation site percentage for the set of 3 or more CpG dinucleotides of below 20.0% indicates a low cancer potential state, a methylation site percentage for the set of 3 or more CpG dinucleotides from 20.0% to 50.0% indicates a moderate cancer potential state, and a methylation site percentage for the set of 3 or more CpG dinucleotides of over 50.0% indicates a high cancer potential state.

In some forms, the set of 3 or more CpG dinucleotides can comprise the CpG dinucleotide corresponding to position 5177 of HPV16. In some forms, the set of 3 or more CpG dinucleotides can further comprise the CpG dinucleotide corresponding to position 6455 of HPV16. In some forms, the set of 3 or more CpG dinucleotides can further comprise the CpG dinucleotides corresponding to positions 3887, 3937, 3941, 5126, 5171, 5600, 5609, 5707, 5724, 5925, and 6365 of HPV16. In some forms, the set of 3 or more CpG dinucleotides can further comprise the CpG dinucleotides corresponding to positions 5615 and 5961 of HPV16. In some forms, the set of 3 or more CpG dinucleotides can further comprise the CpG dinucleotides corresponding to positions 5126, 5171, 5600, 5609, 5707, 5724, 5925, and 6365 of HPV16. In some forms, the set of 3 or more CpG dinucleotides can further comprise the CpG dinucleotides corresponding to positions 3887, 3937, and 3941 of HPV16. In some forms, the set of 3 or more CpG dinucleotides can further comprise the CpG dinucleotides corresponding to positions 5376, 5606, 6387, and 6579 of HPV16. In some forms, the set of 3 or more CpG dinucleotides can further comprise the CpG dinucleotides corresponding to positions 5126, 5171, 5600, 5609, 5707, 5724, 5925, and 6365 of HPV16. In some forms, the set 3 or more CpG dinucleotides can comprise the CpG dinucleotides that correspond to positions 3887, 3937, 3941, 4439, 5126, 5600, 5707, 5724, and 6387 of HPV16. In some forms, the set of 3 or more CpG dinucleotides can further comprise the CpG dinucleotides corresponding to positions 4435 and 6729 of HPV16. In some forms, the set of 3 or more CpG dinucleotides for which the methylation state is determined can comprise the 113 CpG dinucleotides of HPV16.

In some forms, identification of the cancer potential state of the subject can be further based on both cytologic screening and on the methylation state of the set of CpG dinucleotides. In some forms, a cytology assessment of low-grade squamous intraepithelial lesion (LSIL) and a methylation site percentage for the set of 3 or more CpG dinucleotides of below 20.0% indicates a low cancer potential state, a methylation site percentage for the set of 3 or more CpG dinucleotides from 20.0% to 50.0% indicates a moderate cancer potential state, and a methylation site percentage for the set of 3 or more CpG dinucleotides of over 50.0% indicates a high cancer potential state.

In some forms, a cytology assessment of atypical squamous cells (ASC) and a methylation site percentage for the set of 3 or more CpG dinucleotides of below 15.0% indicates a low cancer potential state, a cytology assessment of ASC and a methylation site percentage for the set of 3 or more CpG dinucleotides from 15.0% to 35.0% indicates a moderate cancer potential state, and a cytology assessment of ASC and a methylation site percentage for the set of 3 or more CpG dinucleotides of over 35.0% indicates a high cancer potential state.

In some forms, a cytology assessment of high-grade squamous intraepithelial lesion (HSIL) and a methylation site percentage for the set of 3 or more CpG dinucleotides of below 10.0% indicates a low cancer potential state, a cytology assessment of HSIL and a methylation site percentage for the set of 3 or more CpG dinucleotides from 10.0% to 35.0% indicates a moderate cancer potential state, and a cytology assessment of HSIL and a methylation site percentage for the set of 3 or more CpG dinucleotides of over 35.0% indicates a high cancer potential state.

In some forms, a methylation site percentage for the set of 3 or more CpG dinucleotides of over 37.0% indicates that the subject should be tested further. In some forms, a methylation site percentage for the set of 3 or more CpG dinucleotides of over 37.0% indicates that the subject should be referred for colposcopy. In some forms, a methylation site percentage for the set of 3 or more CpG dinucleotides of up to 37.0% indicates that the subject need not be tested further for at least 6 months.

In some forms, identification of the cancer potential state of the subject can be further based on both histologic screening and on the methylation state of the set of CpG dinucleotides. In some forms, a histology assessment of HPV-associated intraepithelial neoplasia grade 1 and a methylation site percentage for the set of 3 or more CpG dinucleotides of below 20.0% indicates a low cancer potential state, a methylation site percentage for the set of 3 or more CpG dinucleotides from 20.0% to 50.0% indicates a moderate cancer potential state, and a methylation site percentage for the set of 3 or more CpG dinucleotides of over 50.0% indicates a high cancer potential state.

In some forms, a histology assessment of HPV-associated intraepithelial neoplasia grade 2 and a methylation site percentage for the set of 3 or more CpG dinucleotides of below 15.0% indicates a low cancer potential state, a histology assessment of HPV-associated intraepithelial neoplasia grade 2 and a methylation site percentage for the set of 3 or more CpG dinucleotides from 15.0% to 50.0% indicates a moderate cancer potential state, and a histology assessment of HPV-associated intraepithelial neoplasia grade 2 and a methylation site percentage for the set of 3 or more CpG dinucleotides over 35.0% indicates a high cancer potential state.

In some forms, a histology assessment of HPV-associated intraepithelial neoplasia grade 3 and a methylation site percentage for the set of 3 or more CpG dinucleotides of below 35.0% indicates a moderate cancer potential state, and a histology assessment of HPV-associated intraepithelial neoplasia grade 3 and a methylation site percentage for the set of 3 or more CpG dinucleotides over 35.0% indicates a high cancer potential state.

In some forms, identification of the cancer potential state of the subject can be further based on cytologic screening, on histologic screening, and on the methylation state of the set of CpG dinucleotides. In some forms, a cytology assessment of LSIL, a histology assessment of HPV-associated intraepithelial neoplasia grade 1, and a methylation site percentage for the set of 3 or more CpG dinucleotides of below 20.0% indicates a low cancer potential state, a methylation site percentage for the set of 3 or more CpG dinucleotides from 20.0% to 50.0% indicates a moderate cancer potential state, and a methylation site percentage for the set of 3 or more CpG dinucleotides of over 50.0% indicates a high cancer potential state.

In some forms, a cytology assessment of LSIL, a histology assessment of HPV-associated intraepithelial neoplasia grade 2, and a methylation site percentage for the set of 3 or more CpG dinucleotides of below 15.0% indicates a low cancer potential state, a cytology assessment of LSIL, a histology assessment of HPV-associated intraepithelial neoplasia grade 2, and a methylation site percentage for the set of 3 or more CpG dinucleotides from 15.0% to 50.0% indicates a moderate cancer potential state, and a cytology assessment of LSIL, a histology assessment of HPV-associated intraepithelial neoplasia grade 2, and a methylation site percentage for the set of 3 or more CpG dinucleotides of over 50.0% indicates a high cancer potential state.

In some forms, a cytology assessment of LSIL, a histology assessment of HPV-associated intraepithelial neoplasia grade 3, and a methylation site percentage for the set of 3 or more CpG dinucleotides of below 10.0% indicates a low cancer potential state, a cytology assessment of LSIL, a histology assessment of HPV-associated intraepithelial neoplasia grade 3, and a methylation site percentage for the set of 3 or more CpG dinucleotides from 10.0% to 35.0% indicates a moderate cancer potential state, and a cytology assessment of LSIL, a histology assessment of HPV-associated intraepithelial neoplasia grade 3, and a methylation site percentage for the set of 3 or more CpG dinucleotides of over 35.0% indicates a high cancer potential state.

In some forms, a cytology assessment of ASC, a histology assessment of HPV-associated intraepithelial neoplasia grade 1, and a methylation site percentage for the set of 3 or more CpG dinucleotides of below 15.0% indicates a low cancer potential state, a cytology assessment of ASC, a histology assessment of HPV-associated intraepithelial neoplasia grade 1, and a methylation site percentage for the set of 3 or more CpG dinucleotides from 15.0% to 50.0% indicates a moderate cancer potential state, and a cytology assessment of ASC, a histology assessment of HPV-associated intraepithelial neoplasia grade 1, and a methylation site percentage for the set of 3 or more CpG dinucleotides of over 50.0% indicates a high cancer potential state.

In some forms, a cytology assessment of ASC, a histology assessment of HPV-associated intraepithelial neoplasia grade 2, and a methylation site percentage for the set of 3 or more CpG dinucleotides of below 15.0% indicates a low cancer potential state, a cytology assessment of ASC, a histology assessment of HPV-associated intraepithelial neoplasia grade 2, and a methylation site percentage for the set of 3 or more CpG dinucleotides from 15.0% to 35.0% indicates a moderate cancer potential state, and a cytology assessment of ASC, a histology assessment of HPV-associated intraepithelial neoplasia grade 2, and a methylation site percentage for the set of 3 or more CpG dinucleotides of over 35.0% indicates a high cancer potential state.

In some forms, a cytology assessment of ASC, a histology assessment of HPV-associated intraepithelial neoplasia grade 3, and a methylation site percentage for the set of 3 or more CpG dinucleotides of below 35.0% indicates a moderate cancer potential state, and a cytology assessment of ASC, a histology assessment of HPV-associated intraepithelial neoplasia grade 3, and a methylation site percentage for the set of 3 or more CpG dinucleotides of over 35.0% indicates a high cancer potential state.

In some forms, a cytology assessment of HSIL, a histology assessment of HPV-associated intraepithelial neoplasia grade 1, and a methylation site percentage for the set of 3 or more CpG dinucleotides of below 15.0% indicates a low cancer potential state, a cytology assessment of HSIL, a histology assessment of HPV-associated intraepithelial neoplasia grade 1, and a methylation site percentage for the set of 3 or more CpG dinucleotides from 15.0% to 35.0% indicates a moderate cancer potential state, and a cytology assessment of HSIL, a histology assessment of HPV-associated intraepithelial neoplasia grade 1, and a methylation site percentage for the set of 3 or more CpG dinucleotides of over 35.0% indicates a high cancer potential state,

In some forms, a cytology assessment of HSIL, a histology assessment of HPV-associated intraepithelial neoplasia grade 2, and a methylation site percentage for the set of 3 or more CpG dinucleotides of below 10.0% indicates a low cancer potential state, a cytology assessment of HSIL, a histology assessment of HPV-associated intraepithelial neoplasia grade 2, and a methylation site percentage for the set of 3 or more CpG dinucleotides from 10.0% to 35.0% indicates a moderate cancer potential state, and a cytology assessment of HSIL, a histology assessment of HPV-associated intraepithelial neoplasia grade 2, and a methylation site percentage for the set of 3 or more CpG dinucleotides of over 35.0% indicates a high cancer potential state,

In some forms, a cytology assessment of HSIL, a histology assessment of HPV-associated intraepithelial neoplasia grade 3, and a methylation site percentage for the set of 3 or more CpG dinucleotides of below 20.0% indicates a moderate cancer potential state, and a cytology assessment of HSIL, a histology assessment of HPV-associated intraepithelial neoplasia grade 3, and a methylation site percentage for the set of 3 or more CpG dinucleotides of over 20.0% indicates a high cancer potential state.

In some forms, identification of the cancer potential state of the subject can be further based on both histologic screening and on the methylation state of the set of CpG dinucleotides. In some forms, a histology assessment of CIN-1 and a methylation site percentage for the set of 3 or more CpG dinucleotides of below 20.0% indicates a low cancer potential state, a methylation site percentage for the set of 3 or more CpG dinucleotides from 20.0% to 50.0% indicates a moderate cancer potential state, and a methylation site percentage for the set of 3 or more CpG dinucleotides of over 50.0% indicates a high cancer potential state.

In some forms, a histology assessment of CIN-2 and a methylation site percentage for the set of 3 or more CpG dinucleotides of below 15.0% indicates a low cancer potential state, a histology assessment of CIN-2 and a methylation site percentage for the set of 3 or more CpG dinucleotides from 15.0% to 50.0% indicates a moderate cancer potential state, and a histology assessment of CIN-2 and a methylation site percentage for the set of 3 or more CpG dinucleotides over 35.0% indicates a high cancer potential state.

In some forms, a histology assessment of CIN-3 and a methylation site percentage for the set of 3 or more CpG dinucleotides of below 35.0% indicates a moderate cancer potential state, and a histology assessment of CIN-3 and a methylation site percentage for the set of 3 or more CpG dinucleotides over 35.0% indicates a high cancer potential state.

In some forms, identification of the cancer potential state of the subject can be further based on cytologic screening, on histologic screening, and on the methylation state of the set of CpG dinucleotides. In some forms, a cytology assessment of LSIL, a histology assessment of CIN-1, and a methylation site percentage for the set of 3 or more CpG dinucleotides of below 20.0% indicates a low cancer potential state, a methylation site percentage for the set of 3 or more CpG dinucleotides from 20.0% to 50.0% indicates a moderate cancer potential state, and a methylation site percentage for the set of 3 or more CpG dinucleotides of over 50.0% indicates a high cancer potential state.

In some forms, a cytology assessment of LSIL, a histology assessment of CIN-2, and a methylation site percentage for the set of 3 or more CpG dinucleotides of below 15.0% indicates a low cancer potential state, a cytology assessment of LSIL, a histology assessment of CIN-2, and a methylation site percentage for the set of 3 or more CpG dinucleotides from 15.0% to 50.0% indicates a moderate cancer potential state, and a cytology assessment of LSIL, a histology assessment of CIN-2, and a methylation site percentage for the set of 3 or more CpG dinucleotides of over 50.0% indicates a high cancer potential state.

In some forms, a cytology assessment of LSIL, a histology assessment of CIN-3, and a methylation site percentage for the set of 3 or more CpG dinucleotides of below 10.0% indicates a low cancer potential state, a cytology assessment of LSIL, a histology assessment of CIN-3, and a methylation site percentage for the set of 3 or more CpG dinucleotides from 10.0% to 35.0% indicates a moderate cancer potential state, and a cytology assessment of LSIL, a histology assessment of CIN-3, and a methylation site percentage for the set of 3 or more CpG dinucleotides of over 35.0% indicates a high cancer potential state.

In some forms, a cytology assessment of ASC, a histology assessment of CIN-1, and a methylation site percentage for the set of 3 or more CpG dinucleotides of below 15.0% indicates a low cancer potential state, a cytology assessment of ASC, a histology assessment of CIN-1, and a methylation site percentage for the set of 3 or more CpG dinucleotides from 15.0% to 50.0% indicates a moderate cancer potential state, and a cytology assessment of ASC, a histology assessment of CIN-1, and a methylation site percentage for the set of 3 or more CpG dinucleotides of over 50.0% indicates a high cancer potential state.

In some forms, a cytology assessment of ASC, a histology assessment of CIN-2, and a methylation site percentage for the set of 3 or more CpG dinucleotides of below 15.0% indicates a low cancer potential state, a cytology assessment of ASC, a histology assessment of CIN-2, and a methylation site percentage for the set of 3 or more CpG dinucleotides from 15.0% to 35.0% indicates a moderate cancer potential state, and a cytology assessment of ASC, a histology assessment of CIN-2, and a methylation site percentage for the set of 3 or more CpG dinucleotides of over 35.0% indicates a high cancer potential state.

In some forms, a cytology assessment of ASC, a histology assessment of CIN-3, and a methylation site percentage for the set of 3 or more CpG dinucleotides of below 35.0% indicates a moderate cancer potential state, and a cytology assessment of ASC, a histology assessment of CIN-3, and a methylation site percentage for the set of 3 or more CpG dinucleotides of over 35.0% indicates a high cancer potential state.

In some forms, a cytology assessment of HSIL, a histology assessment of CIN-1, and a methylation site percentage for the set of 3 or more CpG dinucleotides of below 15.0% indicates a low cancer potential state, a cytology assessment of HSIL, a histology assessment of CIN-1, and a methylation site percentage for the set of 3 or more CpG dinucleotides from 15.0% to 35.0% indicates a moderate cancer potential state, and a cytology assessment of HSIL, a histology assessment of CIN-1, and a methylation site percentage for the set of 3 or more CpG dinucleotides of over 35.0% indicates a high cancer potential state,

In some forms, a cytology assessment of HSIL, a histology assessment of CIN-2, and a methylation site percentage for the set of 3 or more CpG dinucleotides of below 10.0% indicates a low cancer potential state, a cytology assessment of HSIL, a histology assessment of CIN-2, and a methylation site percentage for the set of 3 or more CpG dinucleotides from 10.0% to 35.0% indicates a moderate cancer potential state, and a cytology assessment of HSIL, a histology assessment of CIN-2, and a methylation site percentage for the set of 3 or more CpG dinucleotides of over 35.0% indicates a high cancer potential state,

In some forms, a cytology assessment of HSIL, a histology assessment of CIN-3, and a methylation site percentage for the set of 3 or more CpG dinucleotides of below 20.0% indicates a moderate cancer potential state, and a cytology assessment of HSIL, a histology assessment of CIN-3, and a methylation site percentage for the set of 3 or more CpG dinucleotides of over 20.0% indicates a high cancer potential state.

Histologic screening can be performed on any suitable sample of any suitable cells and/or tissues. The assessment made can be made in any suitable way and expressed using any known scale, system, nomenclature, etc. For example, histologic screening can identify or determine a grade or stage of a condition being assessed. For example, a grade or stage of HPV-associated lesion, HPV-associated disease, HPV-associated intraepithelial neoplasia, intraepithelial neoplasia, cervical intraepithelial neoplasia, vulvar intraepithelial neoplasia, vaginal intraepithelial neoplasia, penile intraepithelial neoplasia, anal intraepithelial neoplasia, and head and neck intraepithelial neoplasia can be assessed or identified. Examples of some such grades include cervical intraepithelial neoplasia grade 1 (CIN-1), cervical intraepithelial neoplasia grade 2 (CIN-2), and cervical intraepithelial neoplasia grade 3 (CIN-3). As used herein, “HPV-associated” refers to a disease, condition, infection, etc. that is caused by, affected by, and/or occurs in association with an HPV infection.

In some forms, the method can further comprise performing a histologic screen of the subject for cervical cancer potential at an interval based on the identified cancer potential state. In some forms, the method can further comprise creating a record of the identification of the cancer potential state of the subject. In some forms, the method can further comprise testing the subject for cervical cancer potential at an interval based on the identified cancer potential state. In some forms, the method can further comprise testing the subject for cervical cancer potential in a mode or manner based on the identified cancer potential state. In some forms, the method can further comprise treating the subject at an interval based on the identified cancer potential state. In some forms, the method can further comprise treating the subject in a mode or manner based on the identified cancer potential state.

In some forms, the method can further comprise a second determination of the methylation state of a set of CpG dinucleotides in HPV sequences in a sample of the subject, wherein a change or no change in the prognosis of the subject. In some forms, an increase in methylation, a change in methylation pattern (to a more negative pattern), or both indicates a worsening prognosis of the subject. In some forms, a decrease in methylation, a change in methylation pattern (to a more positive pattern), or both indicates an improving prognosis of the subject. In some forms, no significant change in methylation indicates no change in prognosis of the subject. The second determination can be done at any suitable interval of time, for example, 6 months, 12 months, etc. Additional and multiple determinations can be made over time for the same subject. The change in methylation in second and subsequent determinations can be used, for example, to determine what further tests to perform, if any, and at what intervals. For example, a change in methylation to a more negative pattern can indicate that the subject should be referred for colposcopy. As another example, the frequency of cytological re-testing can be determined by the trend of methylation.

In some forms, the determination of methylation state can be performed following a therapeutic intervention, such as chemotherapy, surgery, etc. If the subject has residual HPV infection, the methylation state can be used to determine the frequency and aggressiveness of follow-up treatment and/or testing. For example, a subject with residual infection and a high cancer potential state could benefit from more frequent monitoring. If the HPV DNA methylation analysis showed a low cancer potential state, the lesion might have a good prognosis, e.g., might clear the infection without further intervention.

Also disclosed are methods comprising determining the methylation state of a set of CpG dinucleotides in HPV16 sequences in a sample of a subject and identifying the cancer potential state of the subject based on the methylation state of the set of CpG dinucleotides, where the set comprises the CpG dinucleotides that correspond to positions 3887, 3937, 3941, 4439, 5126, 5600, 5707, 5724, and 6387 of HPV16. In some forms, the set of CpG dinucleotides can further comprise the CpG dinucleotides corresponding to positions 4435 and 6729 of HPV16.

Also disclosed are methods comprising determining the methylation state of a set of CpG dinucleotides in HPV16 sequences in a sample of a subject and identifying the cancer potential state of the subject based on the pattern of methylation of the set of CpG dinucleotides, where a pattern of methylation corresponding to methylation pattern A indicates a low cancer potential state, and where a pattern of methylation corresponding to methylation pattern C indicates a high cancer potential state.

Also disclosed are devices to perform the disclosed assays. Low cost devices are particularly useful. For example, the reactions can use tiny droplets of sample material and reagents, so that a low cost per assay can allow effective clinical implementation the clinic or at the point of care—which is expected to provide strong incentive for gynecologists and other care providers to use it. The instrument can also be made durable (with no mechanical parts, for example), easy to operate (using disposable cartridges, for example), and needing only electricity to operate. For example, the device can have electrical contacts as its sole external requirement. Such features can make the device ideal for implementation in developing countries where it could be linked to existing surveillance programs, such as the HIV surveillance program (HPV and HIV have the same risk factors). Because developing countries cannot afford Pap smear screening programs, cervical cancer remains the leading cause of female cancer deaths. The disclosed methods can also use self-collected clinical samples, to eliminate the need for pelvic examinations, the least acceptable part of Pap testing for patients.

The disclosed method can comprise determining the methylation state of, for example, a set of a desired number of CpG dinucleotides, a set of CpG dinucleotides in HPV16 sequences in a sample of a subject, a set comprising or consisting of a set of a desired number of CpG dinucleotides, a set consisting of a desired number of CpG dinucleotides, a desired number of CpG dinucleotides, a desired number of a set of CpG dinucleotides, a desired number of CpG dinucleotides, a set of a desired number of specific CpG dinucleotides, a set comprising or consisting of a set of a desired number of specific CpG dinucleotides, a set consisting of a desired number of specific CpG dinucleotides, a desired number of specific CpG dinucleotides, a desired number of a set of specific CpG dinucleotides, and/or a desired number of specific CpG dinucleotides.

The desired number of CpG dinucleotides can be, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 94, 96, 97, 98, 99, 100, 101, 102, 103, 104, 105, 106, 107, 108, 109, 110, 111, 112, or 113. The desired number of CpG dinucleotides can be, for example, 1 or more, 2 or more, 3 or more, etc. The desired number of CpG dinucleotides can be, for example, 2 or less, 3 or less, 4 or less, 5 or less, etc. The desired number of CpG dinucleotides can be, for example, at least 1, at least 2, at least 3, etc. It should be understood that, although numerous examples and descriptions of different forms of the disclosed methods state a specific number of CpG dinucleotides (most commonly “3 or more”), this is merely for convenience and by way of example. Any of the other desired number of CpG dinucleotides can be substituted for any of the specific number of CpG dinucleotides recited in examples and descriptions herein.

Thus, for example, the methylation state can be determined of a subset of set of specific CpG dinucleotides where the subset can be a desired number of the specific dinucleotides and the set of specific CpG dinucleotides can be a set of CpG dinucleotides determined to be of significance for assessing the cancer potential state of subjects. The subset in this example can be the only CpG dinucleotides assessed, or other CpG dinucleotides can also be assessed along with the subset of CpG dinucleotides.

The specific CpG dinucleotides can be, for example, CpG dinucleotides that correspond to positions in a nucleic acid, such as an HPV genome, such as the HPV16 genome. Useful CpG positions in the HPV16 genome include, for example, 3887, 3937, 3941, 4016, 4084, 4238, 4247, 4259, 4268, 4275, 4425, 4435, 4439, 4537, 4892, 4904, 4922, 5126, 5171, 5177, 5376, 5600, 5606, 5609, 5615, 5707, 5724, 5925, 5961, 6365, 6387, 6455, 6579, 6648, 6729, 6794, 7032, and 7089; 3887, 3937, 3941, 5126, 5171, 5177, 5376, 5600, 5606, 5609, 5615, 5707, 5724, 5925, 5961, 6365, 6387, 6455, and 6579; 3887, 3937, 3941, 4435, 4439, 5126, 5600, 5707, 5724, 6387, and 6729; and 3887, 3937, 3941, 4435, 4439, 5126, 5171, 5177, 5376, 5600, 5606, 5609, 5615, 5707, 5724, 5925, 5961, 6365, 6387, 6455, and 6579.

The disclosed methods can comprise, for example, identifying the cancer potential state of the subject, identifying the subject as having a cancer potential state of interest, identifying the subject as having a cancer potential state above a threshold, and/or identifying the subject as having a cancer potential state of moderate or above.

The disclosed identifications can be, for example, based on the methylation state of the set of CpG dinucleotides, based on the methylation state of the set of CpG dinucleotides and not based on the methylation state of any other CpG dinucleotides in the HPV16 sequences in the sample, based only on the methylation state of the set of CpG dinucleotides, based essentially on the methylation state of the set of CpG dinucleotides, and/or based on the methylation state essentially of the set of CpG dinucleotides. As used herein, “based essentially on” means that the conclusion or result is based only on the recited elements and any other elements that are not of the same class, type, significance, or a combination. Thus, for example, an identification based essentially on the methylation state of a specific set of specific CpG dinucleotides can be based on the specific set of CpG dinucleotides and other CpG dinucleotides that are not of the same class, type, significance. As used herein, “essentially of” in reference to a set means that the set can include the recited elements and any other elements that are not of the same class, type, significance, or a combination. Thus, for example, an identification based on the methylation state essentially of a set of specific CpG dinucleotides can be based on the specific set of CpG dinucleotides and other CpG dinucleotides that are not of the same class, type, significance.

The disclosed methods and the identifications, cancer potential states, methylation states, methylation patterns, etc. can be used to determine whether, at what interval, and/or in what mode or manner a subject is further tested or re-tested. For example, a methylation site percentage for the set of CpG dinucleotides of over 37.0% can indicate that the subject should be tested further, re-tested, and/or should be referred for colposcopy. As another example, a methylation site percentage for the set of CpG dinucleotides of up to 37.0% can indicate that the subject need not be tested further for at least a certain interval. As another example, identifications, cancer potential states, methylation states, methylation patterns, etc. can be used to determine at what interval a subject should be further tested or re-tested. The interval can be, for example, 3, 4, 5, 6, 7, 8, 9, 10, 11, 12, 13, 14, 15, 16, 17, 18, 19, 20, 21, 22, 23, or 24 months. Generally, a low cancer potential state indicates that a the interval can be longer, while a high cancer potential state indicates a shorter interval or immediate follow-up, treatment, further testing, etc.

In some forms, the disclosed methods can comprise determining the methylation state of a set of CpG dinucleotides in HPV16 sequences in a sample of a subject, wherein the set comprises or consists of a set of 3 or more of the CpG dinucleotides that correspond to positions 3887, 3937, 3941, 4016, 4084, 4238, 4247, 4259, 4268, 4275, 4425, 4435, 4439, 4537, 4892, 4904, 4922, 5126, 5171, 5177, 5376, 5600, 5606, 5609, 5615, 5707, 5724, 5925, 5961, 6365, 6387, 6455, 6579, 6648, 6729, 6794, 7032, and 7089 of HPV16, and identifying the cancer potential state of the subject based on the methylation state of the set of CpG dinucleotides.

In some forms, the disclosed methods can comprise determining the methylation state of a set of CpG dinucleotides in HPV16 sequences in a sample of a subject, wherein the set comprises or consists of a set of 3 or more of the CpG dinucleotides that correspond to positions 3887, 3937, 3941, 4016, 4084, 4238, 4247, 4259, 4268, 4275, 4425, 4435, 4439, 4537, 4892, 4904, 4922, 5126, 5171, 5177, 5376, 5600, 5606, 5609, 5615, 5707, 5724, 5925, 5961, 6365, 6387, 6455, 6579, 6648, 6729, 6794, 7032, and 7089 of HPV16, and identifying the cancer potential state of the subject based on the methylation state of the set of 3 or more CpG dinucleotides.

In some forms, the disclosed methods can comprise determining the methylation state of a set of 3 or more of the CpG dinucleotides that correspond to positions 3887, 3937, 3941, 4016, 4084, 4238, 4247, 4259, 4268, 4275, 4425, 4435, 4439, 4537, 4892, 4904, 4922, 5126, 5171, 5177, 5376, 5600, 5606, 5609, 5615, 5707, 5724, 5925, 5961, 6365, 6387, 6455, 6579, 6648, 6729, 6794, 7032, and 7089 of HPV16, and identifying the cancer potential state of the subject based on the methylation state of the set of CpG dinucleotides.

In some forms, the disclosed methods can comprise determining the methylation state of a set of CpG dinucleotides in HPV16 sequences in a sample of a subject, wherein the set comprises or consists of a set consisting of 3 or more of the CpG dinucleotides that correspond to positions 3887, 3937, 3941, 4016, 4084, 4238, 4247, 4259, 4268, 4275, 4425, 4435, 4439, 4537, 4892, 4904, 4922, 5126, 5171, 5177, 5376, 5600, 5606, 5609, 5615, 5707, 5724, 5925, 5961, 6365, 6387, 6455, 6579, 6648, 6729, 6794, 7032, and 7089 of HPV16, and identifying the cancer potential state of the subject based on the methylation state of the set of CpG dinucleotides.

In some forms, the disclosed methods can comprise determining the methylation state of a set of CpG dinucleotides in HPV16 sequences in a sample of a subject, wherein the set comprises or consists of a set consisting of 3 or more of the CpG dinucleotides that correspond to positions 3887, 3937, 3941, 4016, 4084, 4238, 4247, 4259, 4268, 4275, 4425, 4435, 4439, 4537, 4892, 4904, 4922, 5126, 5171, 5177, 5376, 5600, 5606, 5609, 5615, 5707, 5724, 5925, 5961, 6365, 6387, 6455, 6579, 6648, 6729, 6794, 7032, and 7089 of HPV16, and identifying the cancer potential state of the subject based on the methylation state of the set of 3 or more CpG dinucleotides.

In some forms, the disclosed methods can comprise determining the methylation state of a set consisting of 3 or more of the CpG dinucleotides that correspond to positions 3887, 3937, 3941, 4016, 4084, 4238, 4247, 4259, 4268, 4275, 4425, 4435, 4439, 4537, 4892, 4904, 4922, 5126, 5171, 5177, 5376, 5600, 5606, 5609, 5615, 5707, 5724, 5925, 5961, 6365, 6387, 6455, 6579, 6648, 6729, 6794, 7032, and 7089 of HPV16, and identifying the cancer potential state of the subject based on the methylation state of the set of CpG dinucleotides.

In some forms, the disclosed methods can comprise determining the methylation state of a set of CpG dinucleotides in HPV16 sequences in a sample of a subject, wherein the set comprises or consists of 3 or more of the CpG dinucleotides that correspond to positions 3887, 3937, 3941, 4016, 4084, 4238, 4247, 4259, 4268, 4275, 4425, 4435, 4439, 4537, 4892, 4904, 4922, 5126, 5171, 5177, 5376, 5600, 5606, 5609, 5615, 5707, 5724, 5925, 5961, 6365, 6387, 6455, 6579, 6648, 6729, 6794, 7032, and 7089 of HPV16, and identifying the cancer potential state of the subject based on the methylation state of the set of CpG dinucleotides.

In some forms, the disclosed methods can comprise determining the methylation state of a set of CpG dinucleotides in HPV16 sequences in a sample of a subject, wherein the set comprises or consists of 3 or more of the CpG dinucleotides that correspond to positions 3887, 3937, 3941, 4016, 4084, 4238, 4247, 4259, 4268, 4275, 4425, 4435, 4439, 4537, 4892, 4904, 4922, 5126, 5171, 5177, 5376, 5600, 5606, 5609, 5615, 5707, 5724, 5925, 5961, 6365, 6387, 6455, 6579, 6648, 6729, 6794, 7032, and 7089 of HPV16, and identifying the cancer potential state of the subject based on the methylation state of the 3 or more CpG dinucleotides.

In some forms, the disclosed methods can comprise determining the methylation state of 3 or more of the CpG dinucleotides that correspond to positions 3887, 3937, 3941, 4016, 4084, 4238, 4247, 4259, 4268, 4275, 4425, 4435, 4439, 4537, 4892, 4904, 4922, 5126, 5171, 5177, 5376, 5600, 5606, 5609, 5615, 5707, 5724, 5925, 5961, 6365, 6387, 6455, 6579, 6648, 6729, 6794, 7032, and 7089 of HPV16, and identifying the cancer potential state of the subject based on the methylation state of the 3 or more CpG dinucleotides.

In some forms, the disclosed methods can comprise determining the methylation state of a set of CpG dinucleotides in HPV16 sequences in a sample of a subject, wherein the set comprises or consists of 3 or more of a set of CpG dinucleotides that correspond to positions 3887, 3937, 3941, 4016, 4084, 4238, 4247, 4259, 4268, 4275, 4425, 4435, 4439, 4537, 4892, 4904, 4922, 5126, 5171, 5177, 5376, 5600, 5606, 5609, 5615, 5707, 5724, 5925, 5961, 6365, 6387, 6455, 6579, 6648, 6729, 6794, 7032, and 7089 of HPV16, and identifying the cancer potential state of the subject based on the methylation state of the set of CpG dinucleotides in HPV16 sequences in the sample.

In some forms, the disclosed methods can comprise determining the methylation state of a set of CpG dinucleotides in HPV16 sequences in a sample of a subject, wherein the set comprises or consists of 3 or more of a set of CpG dinucleotides that correspond to positions 3887, 3937, 3941, 4016, 4084, 4238, 4247, 4259, 4268, 4275, 4425, 4435, 4439, 4537, 4892, 4904, 4922, 5126, 5171, 5177, 5376, 5600, 5606, 5609, 5615, 5707, 5724, 5925, 5961, 6365, 6387, 6455, 6579, 6648, 6729, 6794, 7032, and 7089 of HPV16, and identifying the cancer potential state of the subject based on the methylation state of the 3 or more of the set of CpG dinucleotides.

In some forms, the disclosed methods can comprise determining the methylation state of 3 or more of a set of CpG dinucleotides that correspond to positions 3887, 3937, 3941, 4016, 4084, 4238, 4247, 4259, 4268, 4275, 4425, 4435, 4439, 4537, 4892, 4904, 4922, 5126, 5171, 5177, 5376, 5600, 5606, 5609, 5615, 5707, 5724, 5925, 5961, 6365, 6387, 6455, 6579, 6648, 6729, 6794, 7032, and 7089 of HPV16, and identifying the cancer potential state of the subject based on the methylation state of the 3 or more of the set of CpG dinucleotides.

In some forms, the disclosed methods can comprise determining the methylation state of a set of CpG dinucleotides in HPV16 sequences in a sample of a subject, wherein the set comprises or consists of 3 or more CpG dinucleotides that correspond to positions 3887, 3937, 3941, 4016, 4084, 4238, 4247, 4259, 4268, 4275, 4425, 4435, 4439, 4537, 4892, 4904, 4922, 5126, 5171, 5177, 5376, 5600, 5606, 5609, 5615, 5707, 5724, 5925, 5961, 6365, 6387, 6455, 6579, 6648, 6729, 6794, 7032, and 7089 of HPV16, and identifying the cancer potential state of the subject based on the methylation state of the set of CpG dinucleotides.

In some forms, the disclosed methods can comprise determining the methylation state of a set of CpG dinucleotides in HPV16 sequences in a sample of a subject, wherein the set comprises or consists of 3 or more CpG dinucleotides that correspond to positions 3887, 3937, 3941, 4016, 4084, 4238, 4247, 4259, 4268, 4275, 4425, 4435, 4439, 4537, 4892, 4904, 4922, 5126, 5171, 5177, 5376, 5600, 5606, 5609, 5615, 5707, 5724, 5925, 5961, 6365, 6387, 6455, 6579, 6648, 6729, 6794, 7032, and 7089 of HPV16, and identifying the cancer potential state of the subject based on the methylation state of the 3 or more CpG dinucleotides.

In some forms, the disclosed methods can comprise determining the methylation state of 3 or more CpG dinucleotides that correspond to positions 3887, 3937, 3941, 4016, 4084, 4238, 4247, 4259, 4268, 4275, 4425, 4435, 4439, 4537, 4892, 4904, 4922, 5126, 5171, 5177, 5376, 5600, 5606, 5609, 5615, 5707, 5724, 5925, 5961, 6365, 6387, 6455, 6579, 6648, 6729, 6794, 7032, and 7089 of HPV16, and identifying the cancer potential state of the subject based on the methylation state of the 3 or more CpG dinucleotides.

The disclosed methods of HPV DNA methylation testing are also useful for selecting an optimal therapeutic regimen for individual patient lesions. For example, methylation state, the frequency of CpG methylation, the specific pattern of methylation and/or the (relative) abundance of certain pattern(s). This is useful for, for example, selecting non-surgical therapies as alternatives if the methylation assessment indicates that such alternatives would be useful. Examples of such therapies include antiviral drugs, immunotherapies, and DNA methylation inhibitors. On the other hand, the methylation assessment can indicate lesions unlikely to respond well to such therapies, in which case the lesions may be best treated by physical removal, usually by surgery or laser ablation.

The disclosed methods are also useful for monitoring the effectiveness and/or effect of treatment or therapy. For example, a change (or lack of change) in methylation state, pattern, etc. when the subject is tested following and/or during treatment can indicate whether the treatment is having a desired effect and/or whether alternative therapy should be used. For example, an increase in methylation, a change in methylation pattern (to a more negative pattern), or both can indicate that the treatment is not working. As another example, a decrease in methylation, a change in methylation pattern (to a more positive pattern), or both can indicate that the treatment is working As another example, no significant change in methylation can indicate that the treatment is not working or that the treatment is holding the progression of the disease.

As used herein, “methylation state” refers to whether a methylation site is methylated or not methylated. In the context of naturally methylated DNA, methylation state refers to cytosines that can be methylated, usually cytosines present in CpG dinucleotides in a DNA molecule.

As used herein, “cancer potential state” can refer to likely or actual cytology of cells in the subject (for example, Negative, LSIL, ASC, ASCUS, ASC-H, AGC, HSIL), likely or actual histology of cells in the subject (for example, Negative, HPV-associated intraepithelial neoplasia grade 1, HPV-associated intraepithelial neoplasia grade 2, HPV-associated intraepithelial neoplasia grade 3, CIN-1, CIN-2, CIN-3), the risk of cancer progression in the subject (for example, None, Low, Moderate, High), the risk of the subject having or developing cancer (for example, None, Low, Moderate, High), other measures of cancer risk, potential, prognosis, etc., or any combination. The risk of cancer progression is a particularly useful measure of cancer potential state for cervical cancer screening and assessment of potential or actual cervical cancer. Cancer potential state can be based on any one or multiple measures of cancer potential state. Cancer potential state can also include as a factor the likely, possible, or expected time for cancer to develop. A low cancer potential state is an assessment that a subject has no or a low potential for having or developing cancer. Low cancer potential state can be based on any one or multiple measures of cancer potential state. A high cancer potential state is an assessment that a subject has a high potential for having or developing cancer. High cancer potential state can be based on any one or multiple measures of cancer potential state.

The cytological assessment can be, for example, negative, low-grade squamous intraepithelial lesion (LSIL), atypical squamous cells (ASC), atypical squamous cells of undetermined significance (ASCUS), atypical squamous cells unable to exclude HSIL (ASC-H), atypical glandular cells (AGC), and high-grade squamous intraepithelial lesion (HSIL). Although the actual distinction between an assessment of ASCUS and an assessment of ASC-H may not indicate any difference in the risk of cancer progression (because these assessments can be subjective), in standard protocols, patients with an assessment of ASC-H are referred for colposcopy, the same as patients with HSIL. In contrast patients with ASCUS are followed by repeat cytology, HPV DNA testing and/or colposcopy. Adenocarcinoma arises from glandular cells and in theory an assessment of AGC may indicate a precursor of adenocarcinoma. In practice however AGC is usually diagnosed on biopsy as CIN, i.e. a squamous cell abnormality. Patients with AGC are immediately referred for colposcopy, like patients with HSIL. The disclosed methods can be combined with these assessments to indicate whether the subject would or would not likely benefit from standard actions, and thus, whether to take the standard, other, or no action. In general, a cytological assessment of ASC-H or AGC can be combined with the disclosed methylation states, patterns, etc. to modify or identify the cancer potential state in the same manner as described herein for HSIL. Similarly, a cytological assessment of ASCUS can be combined with the disclosed methylation states, patterns, etc. to modify or identify the cancer potential state in the same manner as described herein for ASC.

As used herein, “based on the methylation state” refers to a conclusion, identification, etc. that is made using a methylation state. Thus, for example, identification of the cancer potential state of a subject based on the methylation state of a set of CpG dinucleotides means that the identification is made using the state of methylation of those CpG dinucleotides (as opposed to an identification based on other factors or based on both the methylation of those CpG dinucleotides and other factors).

A CpG dinucleotide that corresponds to a position in a reference nucleic acid or genome refers to the CpG dinucleotides where the cytosine residue appears at the indicated position in the reference nucleic acid or genome. Thus, for example, a CpG dinucleotide that corresponds to position 5600 in the HPV16 genome refers to the CpG dinucleotides where the cytosine residue appears at nucleotide 5600 of the HPV genome. Unless otherwise indicated, the reference genome referred to is the standard reference genome of that organism known in the art and/or to a specific reference genome identified herein. For example, the reference HPV16 genome herein is the W12 isolate of HPV16 (Flores et al., 1999). It is understood that in a particular sequence (in a sample, for example) the actual location of the CpG referred to may differ due to variations in different instances of nucleic acid that may be present in different samples and subjects.

As used herein, “methylation site percentage” refers to the percentage of CpG dinucleotides (of those CpG dinucleotides for which the methylation state is determined) that are methylated (see Table 1). “Genome methylation frequency” refers to the percentage of HPV16 genomes in a sample that are methylated. The genome methylation frequency at a particular site refers to the percentage of HPV16 genomes in a sample that are methylated at the particular CpG measured in a sample (see Table 10). The average Genome methylation frequency per site refers to the average of the percentage of HPV16 genomes in the sample methylated at each particular CpG measured in a sample. “Sample methylation frequency” refers to the percentage of samples with a methylated HPV16 genome (at interrogated CpG site(s)). The sample methylation frequency at a particular site refers to the percentage of samples with an HPV16 genome methylated at a particular CpG measured.

Cytologic screening, cytology screening, and cytological screening, refers to testing, screening, assaying, etc. a cell sample cytological features. For example, Pap smear or Pap tests involve collecting a cervical cell sample and review of the cells for abnormal cytological features. Cytology assessment, cytological assessment, and cytological assessment refers to scoring, grading, identifying, labeling, etc. a sample based on cytologic screening.

Histologic screening, histology screening, and histological screening, refers to studying the microscopic anatomy of cells and tissues a cell sample. For example, analysis of a cervical biopsy for dysplasia and carcinoma is a type of histologic screening. Histology assessment, histological assessment, and histological assessment refers to scoring, grading, identifying, labeling, etc. a sample based on histologic screening.

Testing or treating at an interval refers to the time between different testing and/or treatment. The time between is the interval. Testing or treating in a mode or manner refers to the type of treatment and/or testing or the manner in which treatment and/or testing is performed. For example, different types of tests are different modes and different manners of testing. For example, cytologic screening and histologic screening are different modes and different manners of testing. Different modes and manners of treating and testing can be chosen based on any known or suitable criteria. For example, the mode or manner of treating and/or testing can be based the cancer potential state of a subject.

A pattern of methylation is a pattern of methylation states within or between a genome, gene, nucleic acid, nucleotide segment, set of methylation sites, set of CpG dinucleotides, etc. For example, individual CpG dinucleotides in a set of CpG dinucleotides in nucleic acid in a sample can be methylated or unmethylated and the pattern of the methylation of some CpG dinucleotides and non-methylation of other CpG dinucleotides constitutes a methylation pattern. As another example, percentage of corresponding CpGs in different copies of a nucleic acid in a sample that are methylated constitutes a methylation pattern. Distinct methylation patterns, especially those identified as significant or of interest, can be referred to as methylation profiles.

As used herein, “subject” includes, but is not limited to, animals, plants, bacteria, viruses, parasites and any other organism or entity. The subject can be a vertebrate, more specifically a mammal (e.g., a human, horse, pig, rabbit, dog, sheep, goat, non-human primate, cow, cat, guinea pig or rodent), a fish, a bird or a reptile or an amphibian. The subject can be an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

### Materials

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if an assay is disclosed and discussed and a number of modifications that can be made to a number of molecules and/or methods including the assay are discussed, each and every combination and permutation of assay and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. CpG Dinucleotides in Human Papillomavirus

The HPV16 genome contains 113 CpGs (other types of HPV contain similar numbers of CpGs), and multiple CpGs occur in each gene as well as the LCR. It has been discovered that methylation patterns in all or subsets of these CpGs are correlated with different stages, grades, diagnoses, and prognoses of cervical dysplasia and cervical cancer. Particularly useful subsets of CpGs include CpGs that correspond to positions 3887, 3937, 3941, 4016, 4084, 4238, 4247, 4259, 4268, 4275, 4425, 4435, 4439, 4537, 4892, 4904, 4922, 5126, 5171, 5177, 5376, 5600, 5606, 5609, 5615, 5707, 5724, 5925, 5961, 6365, 6387, 6455, 6579, 6648, 6729, 6794, 7032, and 7089; positions 3887, 3937, 3941, 5126, 5171, 5177, 5376, 5600, 5606, 5609, 5615, 5707, 5724, 5925, 5961, 6365, 6387, 6455, and 6579; positions 3887, 3937, 3941, 4435, 4439, 5126, 5600, 5707, 5724, 6387, and 6729; and positions 3887, 3937, 3941, 4435, 4439, 5126, 5171, 5177, 5376, 5600, 5606, 5609, 5615, 5707, 5724, 5925, 5961, 6365, 6387, 6455, and 6579 in the HPV16 genome. Table 7 shows 19 CpGs that have been discovered to be significant and sufficient for assessing cancer potential state of subjects (number 1 to 19 in first column). The fifth and sixth columns in Table 7 indicate subsets of nine and thirteen CpGs that are most significant. The seventh and eighth columns in Table 7 indicate the nine CpGs that are unique to pattern C and the eleven CpGs that are significant to pattern C.

TABLE 7
Specific CpGs of Diagnostic and Prognostic Significance

| | CpG | | | | | | |
|---|---|---|---|---|---|---|---|
| | Nucle- | CpG | | Most | Most | Unique | Signif- |
| | otide | Gene | Amp- | Signif- | Signif- | to | icant to |
| CpG | Loca- | Loca- | li- | icant | icant | Pattern | Pattern |
| Number | tion | tion | con | Nine | Thirteen | C | C |
| 1 | 3887 | E5 | 11 | | * | * | * |
| 2 | 3937 | E5 | 11 | | * | * | * |
| 3 | 3941 | E5 | 11 | | * | * | * |
| | 4016 | E5 | 12 | | | | |
| | 4084 | E5 | 12 | | | | |
| | 4238 | L2 | 12 | | | * | * |
| | 4247 | L2 | 12 | | | | |
| | 4259 | L2 | 12 | | | | |
| | 4268 | L2 | 12 | | | | |
| | 4275 | L2 | 12 | | | | |
| | 4425 | L2 | 13 | | | | |
| | 4435 | L2 | 13 | | | | * |
| | 4439 | L2 | 13 | | | | * |
| | 4537 | L2 | 13 | | | | |
| | 4892 | L2 | 14 | | | | |
| | 4904 | L2 | 14 | | | | |
| | 4922 | L2 | 14 | | | | |
| 4 | 5126 | L2 | 15 | * | * | * | * |
| 5 | 5171 | L2 | 15 | * | * | | |
| 6 | 5177 | L2 | 15 | * | * | | |
| 7 | 5376 | L2 | 15 | | | | |
| 8 | 5600 | L2/L1 | 16 | * | * | * | * |
| 9 | 5606 | L2/L1 | 16 | | | | |
| 10 | 5609 | L2/L1 | 16 | * | * | | |
| 11 | 5615 | L2/L1 | 16 | | | | |
| 12 | 5707 | L1 | 16 | * | * | * | * |
| 13 | 5724 | L1 | 16 | * | * | * | * |
| 14 | 5925 | L1 | 16 | * | * | | |
| 15 | 5961 | L1 | 16 | | | | |
| 16 | 6365 | L1 | 17 | * | * | | |
| 17 | 6387 | L1 | 17 | | | * | * |
| 18 | 6455 | L1 | 17 | | * | | |
| 19 | 6579 | L1 | 17 | | | | |
| | 6648 | L1 | 18 | | | | |
| | 6729 | L1 | 18 | | | | |
| | 6794 | L1 | 18 | | | | |
| | 7032 | L1 | 18 | | | | |
| | 7089 | L1 | 18 | | | | |

Examples 1 describes methylation patterns that are useful for identifying different stages, grades, diagnoses, and prognoses of cervical dysplasia and cervical cancer. Pattern C is correlated with a high cancer potential state. CpGs unique to patterns C are listed in Table 1. The CpGs defining pattern C have been discovered to be useful for identifying different stages, grades, diagnoses, and prognoses of cervical dysplasia and cervical cancer.

The disclosed CpG dinucleotides can be grouped into different sets based on various criteria. For example, in some forms of the disclosed methods, CpGs 5177, 6455, 5625, and 5961, either alone or in any combination, are particularly useful to include in the set of CpGs assessed or on which identification is based. As another example, CpGs 5126, 5171, 5177, 5600, 5609, 5707, 5724, 5925, and 6365 are useful to include in the set of CpGs assessed or on which identification is based (these are the most significant nine CpGs of the 19 identified CpGs; see Table 7). As another example, CpGs 3887, 3937, 3941, 5126, 5171, 5177, 5600, 5609, 5707, 5724, 5925, 6365, and 6455 are useful to include in the set of CpGs assessed or on which identification is based (these are the most significant thirteen CpGs of the 19 identified CpGs; see Table 7). As another example, CpGs 5171, 5177, 5376, 5606, 5609, 5615, 5925, 5961, 6365, 6455, and 6579 are useful to include in the set of CpGs assessed or on which identification is based. As another example, CpGs 3887, 3937, 3941, 4439, 5126, 5600, 5707, 5724, and 6387 are useful to include in the set of CpGs assessed or on which identification is based (this is the subset of nine CpGs unique to HPV methylation pattern C; see Table 1). As another example, CpGs 3887, 3937, 3941, 4435, 4439, 5126, 5600, 5707, 5724, 6387, and 6729 are useful to include in the set of CpGs assessed or on which identification is based.

In some forms, the set of CpG dinucleotides can comprise the CpG dinucleotide corresponding to position 5177 of HPV16. In some forms, the set of CpG dinucleotides can further comprise the CpG dinucleotide corresponding to position 6455 of HPV16. In some forms, the set of CpG dinucleotides can further comprise the CpG dinucleotides corresponding to positions 3887, 3937, 3941, 5126, 5171, 5600, 5609, 5707, 5724, 5925, and 6365 of HPV16. In some forms, the set of CpG dinucleotides can further comprise the CpG dinucleotides corresponding to positions 5615 and 5961 of HPV16. In some forms, the set of CpG dinucleotides can further comprise the CpG dinucleotides corresponding to positions 5126, 5171, 5600, 5609, 5707, 5724, 5925, and 6365 of HPV16. In some forms, the set of CpG dinucleotides can further comprise the CpG dinucleotides corresponding to positions 3887, 3937, and 3941 of HPV16. In some forms, the set of CpG dinucleotides can further comprise the CpG dinucleotides corresponding to positions 5376, 5606, 6387, and 6579 of HPV16. In some forms, the set of CpG dinucleotides can further comprise the CpG dinucleotides corresponding to positions 5126, 5171, 5600, 5609, 5707, 5724, 5925, and 6365 of HPV16. In some forms, the set of CpG dinucleotides can comprise the CpG dinucleotides that correspond to positions 3887, 3937, 3941, 4439, 5126, 5600, 5707, 5724, and 6387 of HPV16. In some forms, the set of CpG dinucleotides can further comprise the CpG dinucleotides corresponding to positions 4435 and 6729 of HPV16. In some forms, the set of CpG dinucleotides for which the methylation state is determined can comprise the 113 CpG dinucleotides of HPV16.

### B. Samples

For the disclosed methods, samples generally can be collected and/or obtained in any of the manners and modes in which samples are collected and obtained for Pap testing. Samples can also be collected in other manners and be in other forms. For example, biopsies, bodily fluids, washes, etc. can be used. Samples can be obtained form any source of location. For the disclosed methods, samples generally can be cervical samples, uterine samples, vaginal samples, vulvar samples, anal samples, rectal samples, oral samples, or nasal samples.

By “sample” is intended any sampling of cells, tissues, or bodily fluids. Examples of such samples include but are not limited to blood, lymph, urine, gynecological fluids, biopsies, and smears. Bodily fluids can include blood, urine, saliva, nipple aspirates, or any other bodily secretion or derivative thereof. Blood can include whole blood, plasma, serum, or any derivative of blood. The sample can comprise cervical cells, particularly cervical cells from swabs and washings or cervical tissue from a biopsy. Samples can be obtained from a subject by a variety of techniques including, for example, by scraping, washing, or swabbing an area, by using a needle to aspirate bodily fluids, or by removing a tissue sample (i.e., biopsy). Methods for collecting various samples are well known in the art. Fixative and staining solutions can be applied to the cells or tissues for preserving the specimen and for facilitating examination. Body samples, particularly cervical cell or tissue samples, can be transferred to a glass slide for viewing under magnification.

### C. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for assessing cancer risk, the kit comprising HPV amplification primers. The kits also can contain, for example, reagents for bisulfite treatment, DNA sequencing, DNA amplification, etc.

The disclosed kits can also include pods or modules for carrying out one or more of the disclosed methods in a device, machine, and/or system designed to receive such pods or modules.

### D. Mixtures

Disclosed are mixtures formed by performing or preparing to perform the disclosed method. For example, disclosed are mixtures comprising a sample and HPV amplification primers, a cervical sample and HPV amplification primers, a sample and bisulfite reagents, etc.

Whenever the method involves mixing or bringing into contact compositions or components or reagents, performing the method creates a number of different mixtures. For example, if the method includes 3 mixing steps, after each one of these steps a unique mixture is formed if the steps are performed separately. In addition, a mixture is formed at the completion of all of the steps regardless of how the steps were performed. The present disclosure contemplates these mixtures, obtained by the performance of the disclosed methods as well as mixtures containing any disclosed reagent, composition, or component, for example, disclosed herein.

### E. Systems

Disclosed are systems useful for performing, or aiding in the performance of, the disclosed method. Systems generally comprise combinations of articles of manufacture such as structures, machines, devices, and the like, and compositions, compounds, materials, and the like. Such combinations that are disclosed or that are apparent from the disclosure are contemplated. For example, disclosed and contemplated are systems comprising a device for processing nucleic acid samples and detecting the methylation state of nucleic acids and a device for recording and/or assessing the methylation state. As another example, disclosed and contemplated are systems comprising a device for processing nucleic acid samples and detecting the methylation state of nucleic acids and nucleic acid samples.

### F. Data Structures and Computer Control

Disclosed are data structures used in, generated by, or generated from, the disclosed method. Data structures generally are any form of data, information, and/or objects collected, organized, stored, and/or embodied in a composition or medium. For example, a methylation pattern, methylation profile, or set of methylation states stored in electronic form, such as in RAM or on a storage disk, is a type of data structure.

The disclosed method, or any part thereof or preparation therefor, can be controlled, managed, or otherwise assisted by computer control. Such computer control can be accomplished by a computer controlled process or method, can use and/or generate data structures, and can use a computer program. Such computer control, computer controlled processes, data structures, and computer programs are contemplated and should be understood to be disclosed herein.

### Uses

The disclosed methods and compositions are applicable to numerous areas including, but not limited to, assessment of nucleic acids, nucleic acid samples, and cancer risk. Other uses include determination of methylation state, patterns, and profiles and identification of cancer potential states. Other uses are disclosed, apparent from the disclosure, and/or will be understood by those in the art.

### Methods

Disclosed are methods for assessing cancer potential, state, stage, risk of progression, prognosis, etc. of a subject based on determining the methylation state of human papillomavirus (HPV) in a sample from the subject. The cancers assessed generally can be cancers associated with or caused by HPV. For example, cervical cancer, vulvar cancer, vaginal cancer, penile cancer, anal cancer, and head and neck cancer can be associated with HPV. It has been discovered that certain patterns, profiles, and sets of methylation of HPV genomes are correlated with different cancer potential, state, stage, risk of progression, prognosis, etc.

Disclosed are methods comprising determining the methylation state of a set of CpG dinucleotides in HPV sequences in a sample of a subject and identifying the cancer potential state of the subject based on the methylation state of the set of 3 or more CpG dinucleotides. The set can comprise a set of 3 or more of the CpG dinucleotides that correspond to positions 3887, 3937, 3941, 4016, 4084, 4238, 4247, 4259, 4268, 4275, 4425, 4435, 4439, 4537, 4892, 4904, 4922, 5126, 5171, 5177, 5376, 5600, 5606, 5609, 5615, 5707, 5724, 5925, 5961, 6365, 6387, 6455, 6579, 6648, 6729, 6794, 7032, and 7089 of HPV16. The HPV sequences in the sample can be, for example, HPV16 sequences or HPV18 sequences.

In some forms, a methylation site percentage for the set of 3 or more CpG dinucleotides of over 37.0% indicates that the subject should be tested further. In some forms, a methylation site percentage for the set of 3 or more CpG dinucleotides of up to 37.0% indicates that the subject need not be tested further for at least 6 months.

In some forms, the method can further comprise performing a histologic screen of the subject for cervical cancer potential at an interval based on the identified cancer potential state. In some forms, the method can further comprise creating a record of the identification of the cancer potential state of the subject. In some forms, the method can further comprise testing the subject for cervical cancer potential at an interval based on the identified cancer potential state. In some forms, the method can further comprise testing the subject for cervical cancer potential in a mode or manner based on the identified cancer potential state. In some forms, the method can further comprise treating the subject at an interval based on the identified cancer potential state. In some forms, the method can further comprise treating the subject in a mode or manner based on the identified cancer potential state.

In some forms, the method can further comprise a second determination of the methylation state of a set of CpG dinucleotides in HPV sequences in a sample of the subject, wherein a change or no change in the prognosis of the subject. In some forms, an increase in methylation, a change in methylation pattern (to a more negative pattern), or both indicates a worsening prognosis of the subject. In some forms, a decrease in methylation, a change in methylation pattern (to a more positive pattern), or both indicates an improving prognosis of the subject. In some forms, no significant change in methylation indicates no change in prognosis of the subject. The second determination can be done at any suitable interval of time, for example, 6 months, 12 months, etc. Additional and multiple determinations can be made over time for the same subject. The change in methylation in second and subsequent determinations can be used, for example, to determine what further tests to perform, if any, and at what intervals. For example, a change in methylation to a more negative pattern can indicate that the subject should be referred for colposcopy. As another example, the frequency of cytological re-testing can be determined by the trend of methylation.

In some forms, the determination of methylation state can be performed following a therapeutic intervention, such as chemotherapy, surgery, etc. If the subject has residual HPV infection, the methylation state can be used to determine the frequency and aggressiveness of follow-up treatment and/or testing. For example, a subject with residual infection and a high cancer potential state could benefit from more frequent monitoring. If the HPV DNA methylation analysis showed a low cancer potential state, the lesion might have a good prognosis, e.g., might clear the infection without further intervention.

Also disclosed are methods comprising determining the methylation state of a set of CpG dinucleotides in HPV16 sequences in a sample of a subject and identifying the cancer potential state of the subject based on the methylation state of the set of CpG dinucleotides, where the set comprises the CpG dinucleotides that correspond to positions 3887, 3937, 3941, 4439, 5126, 5600, 5707, 5724, and 6387 of HPV16. In some forms, the set of CpG dinucleotides can further comprise the CpG dinucleotides corresponding to positions 4435 and 6729 of HPV16.

Also disclosed are methods comprising determining the methylation state of a set of CpG dinucleotides in HPV16 sequences in a sample of a subject and identifying the cancer potential state of the subject based on the pattern of methylation of the set of CpG dinucleotides, where a pattern of methylation corresponding to methylation pattern A indicates a low cancer potential state, and where a pattern of methylation corresponding to methylation pattern C indicates a high cancer potential state.

In some forms, the disclosed methods can comprise determining the methylation state of a set of CpG dinucleotides in HPV16 sequences in a sample of a subject, wherein the set comprises or consists of a set of 3 or more of the CpG dinucleotides that correspond to positions 3887, 3937, 3941, 4016, 4084, 4238, 4247, 4259, 4268, 4275, 4425, 4435, 4439, 4537, 4892, 4904, 4922, 5126, 5171, 5177, 5376, 5600, 5606, 5609, 5615, 5707, 5724, 5925, 5961, 6365, 6387, 6455, 6579, 6648, 6729, 6794, 7032, and 7089 of HPV16, and identifying the cancer potential state of the subject based on the methylation state of the set of CpG dinucleotides.

In some forms, the disclosed methods can comprise determining the methylation state of a set of CpG dinucleotides in HPV16 sequences in a sample of a subject, wherein the set comprises or consists of a set of 3 or more of the CpG dinucleotides that correspond to positions 3887, 3937, 3941, 4016, 4084, 4238, 4247, 4259, 4268, 4275, 4425, 4435, 4439, 4537, 4892, 4904, 4922, 5126, 5171, 5177, 5376, 5600, 5606, 5609, 5615, 5707, 5724, 5925, 5961, 6365, 6387, 6455, 6579, 6648, 6729, 6794, 7032, and 7089 of HPV16, and identifying the cancer potential state of the subject based on the methylation state of the set of 3 or more CpG dinucleotides.

In some forms, the disclosed methods can comprise determining the methylation state of a set of 3 or more of the CpG dinucleotides that correspond to positions 3887, 3937, 3941, 4016, 4084, 4238, 4247, 4259, 4268, 4275, 4425, 4435, 4439, 4537, 4892, 4904, 4922, 5126, 5171, 5177, 5376, 5600, 5606, 5609, 5615, 5707, 5724, 5925, 5961, 6365, 6387, 6455, 6579, 6648, 6729, 6794, 7032, and 7089 of HPV16, and identifying the cancer potential state of the subject based on the methylation state of the set of CpG dinucleotides.

In some forms, the disclosed methods can comprise determining the methylation state of a set of CpG dinucleotides in HPV16 sequences in a sample of a subject, wherein the set comprises or consists of a set consisting of 3 or more of the CpG dinucleotides that correspond to positions 3887, 3937, 3941, 4016, 4084, 4238, 4247, 4259, 4268, 4275, 4425, 4435, 4439, 4537, 4892, 4904, 4922, 5126, 5171, 5177, 5376, 5600, 5606, 5609, 5615, 5707, 5724, 5925, 5961, 6365, 6387, 6455, 6579, 6648, 6729, 6794, 7032, and 7089 of HPV16, and identifying the cancer potential state of the subject based on the methylation state of the set of CpG dinucleotides.

In some forms, the disclosed methods can comprise determining the methylation state of a set of CpG dinucleotides in HPV16 sequences in a sample of a subject, wherein the set comprises or consists of a set consisting of 3 or more of the CpG dinucleotides that correspond to positions 3887, 3937, 3941, 4016, 4084, 4238, 4247, 4259, 4268, 4275, 4425, 4435, 4439, 4537, 4892, 4904, 4922, 5126, 5171, 5177, 5376, 5600, 5606, 5609, 5615, 5707, 5724, 5925, 5961, 6365, 6387, 6455, 6579, 6648, 6729, 6794, 7032, and 7089 of HPV16, and identifying the cancer potential state of the subject based on the methylation state of the set of 3 or more CpG dinucleotides.

In some forms, the disclosed methods can comprise determining the methylation state of a set consisting of 3 or more of the CpG dinucleotides that correspond to positions 3887, 3937, 3941, 4016, 4084, 4238, 4247, 4259, 4268, 4275, 4425, 4435, 4439, 4537, 4892, 4904, 4922, 5126, 5171, 5177, 5376, 5600, 5606, 5609, 5615, 5707, 5724, 5925, 5961, 6365, 6387, 6455, 6579, 6648, 6729, 6794, 7032, and 7089 of HPV16, and identifying the cancer potential state of the subject based on the methylation state of the set of CpG dinucleotides.

In some forms, the disclosed methods can comprise determining the methylation state of a set of CpG dinucleotides in HPV16 sequences in a sample of a subject, wherein the set comprises or consists of 3 or more of the CpG dinucleotides that correspond to positions 3887, 3937, 3941, 4016, 4084, 4238, 4247, 4259, 4268, 4275, 4425, 4435, 4439, 4537, 4892, 4904, 4922, 5126, 5171, 5177, 5376, 5600, 5606, 5609, 5615, 5707, 5724, 5925, 5961, 6365, 6387, 6455, 6579, 6648, 6729, 6794, 7032, and 7089 of HPV16, and identifying the cancer potential state of the subject based on the methylation state of the set of CpG dinucleotides.

In some forms, the disclosed methods can comprise determining the methylation state of a set of CpG dinucleotides in HPV16 sequences in a sample of a subject, wherein the set comprises or consists of 3 or more of the CpG dinucleotides that correspond to positions 3887, 3937, 3941, 4016, 4084, 4238, 4247, 4259, 4268, 4275, 4425, 4435, 4439, 4537, 4892, 4904, 4922, 5126, 5171, 5177, 5376, 5600, 5606, 5609, 5615, 5707, 5724, 5925, 5961, 6365, 6387, 6455, 6579, 6648, 6729, 6794, 7032, and 7089 of HPV16, and identifying the cancer potential state of the subject based on the methylation state of the 3 or more CpG dinucleotides.

In some forms, the disclosed methods can comprise determining the methylation state of 3 or more of the CpG dinucleotides that correspond to positions 3887, 3937, 3941, 4016, 4084, 4238, 4247, 4259, 4268, 4275, 4425, 4435, 4439, 4537, 4892, 4904, 4922, 5126, 5171, 5177, 5376, 5600, 5606, 5609, 5615, 5707, 5724, 5925, 5961, 6365, 6387, 6455, 6579, 6648, 6729, 6794, 7032, and 7089 of HPV16, and identifying the cancer potential state of the subject based on the methylation state of the 3 or more CpG dinucleotides.

In some forms, the disclosed methods can comprise determining the methylation state of a set of CpG dinucleotides in HPV16 sequences in a sample of a subject, wherein the set comprises or consists of 3 or more of a set of CpG dinucleotides that correspond to positions 3887, 3937, 3941, 4016, 4084, 4238, 4247, 4259, 4268, 4275, 4425, 4435, 4439, 4537, 4892, 4904, 4922, 5126, 5171, 5177, 5376, 5600, 5606, 5609, 5615, 5707, 5724, 5925, 5961, 6365, 6387, 6455, 6579, 6648, 6729, 6794, 7032, and 7089 of HPV16, and identifying the cancer potential state of the subject based on the methylation state of the set of CpG dinucleotides in HPV16 sequences in the sample.

In some forms, the disclosed methods can comprise determining the methylation state of a set of CpG dinucleotides in HPV16 sequences in a sample of a subject, wherein the set comprises or consists of 3 or more of a set of CpG dinucleotides that correspond to positions 3887, 3937, 3941, 4016, 4084, 4238, 4247, 4259, 4268, 4275, 4425, 4435, 4439, 4537, 4892, 4904, 4922, 5126, 5171, 5177, 5376, 5600, 5606, 5609, 5615, 5707, 5724, 5925, 5961, 6365, 6387, 6455, 6579, 6648, 6729, 6794, 7032, and 7089 of HPV16, and identifying the cancer potential state of the subject based on the methylation state of the 3 or more of the set of CpG dinucleotides.

In some forms, the disclosed methods can comprise determining the methylation state of 3 or more of a set of CpG dinucleotides that correspond to positions 3887, 3937, 3941, 4016, 4084, 4238, 4247, 4259, 4268, 4275, 4425, 4435, 4439, 4537, 4892, 4904, 4922, 5126, 5171, 5177, 5376, 5600, 5606, 5609, 5615, 5707, 5724, 5925, 5961, 6365, 6387, 6455, 6579, 6648, 6729, 6794, 7032, and 7089 of HPV16, and identifying the cancer potential state of the subject based on the methylation state of the 3 or more of the set of CpG dinucleotides.

In some forms, the disclosed methods can comprise determining the methylation state of a set of CpG dinucleotides in HPV16 sequences in a sample of a subject, wherein the set comprises or consists of 3 or more CpG dinucleotides that correspond to positions 33887, 3937, 3941, 4016, 4084, 4238, 4247, 4259, 4268, 4275, 4425, 4435, 4439, 4537, 4892, 4904, 4922, 5126, 5171, 5177, 5376, 5600, 5606, 5609, 5615, 5707, 5724, 5925, 5961, 6365, 6387, 6455, 6579, 6648, 6729, 6794, 7032, and 7089 of HPV16, and identifying the cancer potential state of the subject based on the methylation state of the set of CpG dinucleotides.

In some forms, the disclosed methods can comprise determining the methylation state of a set of CpG dinucleotides in HPV16 sequences in a sample of a subject, wherein the set comprises or consists of 3 or more CpG dinucleotides that correspond to positions 3887, 3937, 3941, 4016, 4084, 4238, 4247, 4259, 4268, 4275, 4425, 4435, 4439, 4537, 4892, 4904, 4922, 5126, 5171, 5177, 5376, 5600, 5606, 5609, 5615, 5707, 5724, 5925, 5961, 6365, 6387, 6455, 6579, 6648, 6729, 6794, 7032, and 7089 of HPV16, and identifying the cancer potential state of the subject based on the methylation state of the 3 or more CpG dinucleotides.

In some forms, the disclosed methods can comprise determining the methylation state of 3 or more CpG dinucleotides that correspond to positions 3887, 3937, 3941, 4016, 4084, 4238, 4247, 4259, 4268, 4275, 4425, 4435, 4439, 4537, 4892, 4904, 4922, 5126, 5171, 5177, 5376, 5600, 5606, 5609, 5615, 5707, 5724, 5925, 5961, 6365, 6387, 6455, 6579, 6648, 6729, 6794, 7032, and 7089 of HPV16, and identifying the cancer potential state of the subject based on the methylation state of the 3 or more CpG dinucleotides.

### A. Determining Methylation States

The disclosed method can comprise determining the methylation state of, for example, a set of a desired number of CpG dinucleotides, a set of CpG dinucleotides in HPV16 sequences in a sample of a subject, a set comprising or consisting of a set of a desired number of CpG dinucleotides, a set consisting of a desired number of CpG dinucleotides, a desired number of CpG dinucleotides, a desired number of a set of CpG dinucleotides, a desired number of CpG dinucleotides, a set of a desired number of specific CpG dinucleotides, a set comprising or consisting of a set of a desired number of specific CpG dinucleotides, a set consisting of a desired number of specific CpG dinucleotides, a desired number of specific CpG dinucleotides, a desired number of a set of specific CpG dinucleotides, and/or a desired number of specific CpG dinucleotides.

The desired number of CpG dinucleotides can be, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 94, 96, 97, 98, 99, 100, 101, 102, 103, 104, 105, 106, 107, 108, 109, 110, 111, 112, or 113.

Thus, for example, the methylation state can be determined of a subset of set of specific CpG dinucleotides where the subset can be a desired number of the specific dinucleotides and the set of specific CpG dinucleotides can be a set of CpG dinucleotides determined to be of significance for assessing the cancer potential state of subjects. The subset in this example can be the only CpG dinucleotides assessed, or other CpG dinucleotides can also be assessed along with the subset of CpG dinucleotides.

The specific CpG dinucleotides can be, for example, CpG dinucleotides that correspond to positions in a nucleic acid, such as an HPV genome, such as the HPV16 genome. Useful CpG positions in the HPV16 genome include, for example, 3887, 3937, 3941, 4016, 4084, 4238, 4247, 4259, 4268, 4275, 4425, 4435, 4439, 4537, 4892, 4904, 4922, 5126, 5171, 5177, 5376, 5600, 5606, 5609, 5615, 5707, 5724, 5925, 5961, 6365, 6387, 6455, 6579, 6648, 6729, 6794, 7032, and 7089; 3887, 3937, 3941, 5126, 5171, 5177, 5376, 5600, 5606, 5609, 5615, 5707, 5724, 5925, 5961, 6365, 6387, 6455, and 6579; 3887, 3937, 3941, 4435, 4439, 5126, 5600, 5707, 5724, 6387, and 6729; and 3887, 3937, 3941, 4435, 4439, 5126, 5171, 5177, 5376, 5600, 5606, 5609, 5615, 5707, 5724, 5925, 5961, 6365, 6387, 6455, and 6579.

The methylation state of methylation sites can be determined using any known or suitable method or techniques. For example, methylation states can be determined by DNA sequencing following bisulfite treatment of the nucleic acid.

### B. Identifying Cancer Potential States and Risk of Cancer Progression

Cancer potential states of subjects can be identified based on the disclosed HPV methylation patterns, profiles, and percentages. Identification can also be based on other factors, features, and assays. The disclosed methods can comprise, for example, identifying the cancer potential state of the subject, identifying the subject as having a cancer potential state of interest, identifying the subject as having a cancer potential state above a threshold, and/or identifying the subject as having a cancer potential state of moderate or above.

The disclosed identifications can be, for example, based on the methylation state of the set of CpG dinucleotides, based on the methylation state of the set of CpG dinucleotides and not based on the methylation state of any other CpG dinucleotides in the HPV16 sequences in the sample, based only on the methylation state of the set of CpG dinucleotides, based essentially on the methylation state of the set of CpG dinucleotides, and/or based on the methylation state essentially of the set of CpG dinucleotides. As used herein, “based essentially on” means that the conclusion or result is based only on the recited elements and any other elements that are not of the same class, type, significance, or a combination. Thus, for example, an identification based essentially on the methylation state of a specific set of specific CpG dinucleotides can be based on the specific set of CpG dinucleotides and other CpG dinucleotides that are not of the same class, type, significance. As used herein, “essentially of” in reference to a set means that the set can include the recited elements and any other elements that are not of the same class, type, significance, or a combination. Thus, for example, an identification based on the methylation state essentially of a set of specific CpG dinucleotides can be based on the specific set of CpG dinucleotides and other CpG dinucleotides that are not of the same class, type, significance.

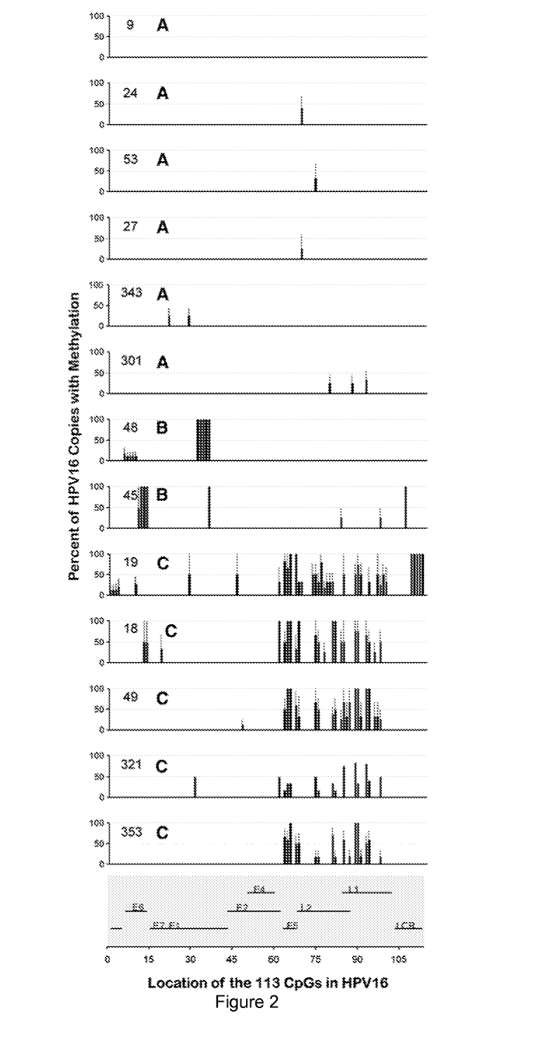

A variety methylation patterns and measures of methylation can be used for identifying the cancer potential state. For example, methylation site percentage, genome methylation frequency, genome methylation frequency at a particular site, average genome methylation frequency per site, sample methylation frequency, sample methylation frequency at a particular site can be used as measures of methylation for identifying the cancer potential state. FIGS. 2, 3, 4, 6, and 7 and Tables 6, 7, 8, 9, and 10 show different methylation patterns and different correlations of methylation states with cancer potential stages for cervical cancer. Any of these and any combination of these can be used with the disclosed methods.

In some forms, a methylation site percentage for the set of 3 or more CpG dinucleotides of below 20.0% indicates a low cancer potential state, a methylation site percentage for the set of 3 or more CpG dinucleotides from 20.0% to 50.0% indicates a moderate cancer potential state, and a methylation site percentage for the set of 3 or more CpG dinucleotides of over 50.0% indicates a high cancer potential state.

In some forms, identification of the cancer potential state of the subject can be further based on both cytologic screening and on the methylation state of the set of CpG dinucleotides. In some forms, a cytology assessment of low-grade squamous intraepithelial lesion (LSIL) and a methylation site percentage for the set of 3 or more CpG dinucleotides of below 20.0% indicates a low cancer potential state, a methylation site percentage for the set of 3 or more CpG dinucleotides from 20.0% to 50.0% indicates a moderate cancer potential state, and a methylation site percentage for the set of 3 or more CpG dinucleotides of over 50.0% indicates a high cancer potential state.

In some forms, a cytology assessment of atypical squamous cells (ASC) and a methylation site percentage for the set of 3 or more CpG dinucleotides of below 15.0% indicates a low cancer potential state, a cytology assessment of ASC and a methylation site percentage for the set of 3 or more CpG dinucleotides from 15.0% to 35.0% indicates a moderate cancer potential state, and a cytology assessment of ASC and a methylation site percentage for the set of 3 or more CpG dinucleotides of over 35.0% indicates a high cancer potential state.

In some forms, a cytology assessment of high-grade squamous intraepithelial lesion (HSIL) and a methylation site percentage for the set of 3 or more CpG dinucleotides of below 10.0% indicates a low cancer potential state, a cytology assessment of HSIL and a methylation site percentage for the set of 3 or more CpG dinucleotides from 10.0% to 35.0% indicates a moderate cancer potential state, and a cytology assessment of HSIL and a methylation site percentage for the set of 3 or more CpG dinucleotides of over 35.0% indicates a high cancer potential state.

In some forms, a methylation site percentage for the set of 3 or more CpG dinucleotides of over 37.0% indicates that the subject should be tested further. In some forms, a methylation site percentage for the set of 3 or more CpG dinucleotides of over 37.0% indicates that the subject should be referred for colposcopy. In some forms, a methylation site percentage for the set of 3 or more CpG dinucleotides of up to 37.0% indicates that the subject need not be tested further for at least 6 months.

In some forms, identification of the cancer potential state of the subject can be further based on both histologic screening and on the methylation state of the set of CpG dinucleotides. In some forms, a histology assessment of CIN-1 and a methylation site percentage for the set of 3 or more CpG dinucleotides of below 20.0% indicates a low cancer potential state, a methylation site percentage for the set of 3 or more CpG dinucleotides from 20.0% to 50.0% indicates a moderate cancer potential state, and a methylation site percentage for the set of 3 or more CpG dinucleotides of over 50.0% indicates a high cancer potential state.

In some forms, a histology assessment of CIN-2 and a methylation site percentage for the set of 3 or more CpG dinucleotides of below 15.0% indicates a low cancer potential state, a histology assessment of CIN-2 and a methylation site percentage for the set of 3 or more CpG dinucleotides from 15.0% to 50.0% indicates a moderate cancer potential state, and a histology assessment of CIN-2 and a methylation site percentage for the set of 3 or more CpG dinucleotides over 35.0% indicates a high cancer potential state.

In some forms, a histology assessment of CIN-3 and a methylation site percentage for the set of 3 or more CpG dinucleotides of below 35.0% indicates a moderate cancer potential state, and a histology assessment of CIN-3 and a methylation site percentage for the set of 3 or more CpG dinucleotides over 35.0% indicates a high cancer potential state.

In some forms, identification of the cancer potential state of the subject can be further based on cytologic screening, on histologic screening, and on the methylation state of the set of CpG dinucleotides. In some forms, a cytology assessment of LSIL, a histology assessment of CIN-1, and a methylation site percentage for the set of 3 or more CpG dinucleotides of below 20.0% indicates a low cancer potential state, a methylation site percentage for the set of 3 or more CpG dinucleotides from 20.0% to 50.0% indicates a moderate cancer potential state, and a methylation site percentage for the set of 3 or more CpG dinucleotides of over 50.0% indicates a high cancer potential state.

In some forms, a cytology assessment of LSIL, a histology assessment of CIN-2, and a methylation site percentage for the set of 3 or more CpG dinucleotides of below 15.0% indicates a low cancer potential state, a cytology assessment of LSIL, a histology assessment of CIN-2, and a methylation site percentage for the set of 3 or more CpG dinucleotides from 15.0% to 50.0% indicates a moderate cancer potential state, and a cytology assessment of LSIL, a histology assessment of CIN-2, and a methylation site percentage for the set of 3 or more CpG dinucleotides of over 50.0% indicates a high cancer potential state.

In some forms, a cytology assessment of LSIL, a histology assessment of CIN-3, and a methylation site percentage for the set of 3 or more CpG dinucleotides of below 10.0% indicates a low cancer potential state, a cytology assessment of LSIL, a histology assessment of CIN-3, and a methylation site percentage for the set of 3 or more CpG dinucleotides from 10.0% to 35.0% indicates a moderate cancer potential state, and a cytology assessment of LSIL, a histology assessment of CIN-3, and a methylation site percentage for the set of 3 or more CpG dinucleotides of over 35.0% indicates a high cancer potential state.

In some forms, a cytology assessment of ASC, a histology assessment of CIN-1, and a methylation site percentage for the set of 3 or more CpG dinucleotides of below 15.0% indicates a low cancer potential state, a cytology assessment of ASC, a histology assessment of CIN-1, and a methylation site percentage for the set of 3 or more CpG dinucleotides from 15.0% to 50.0% indicates a moderate cancer potential state, and a cytology assessment of ASC, a histology assessment of CIN-1, and a methylation site percentage for the set of 3 or more CpG dinucleotides of over 50.0% indicates a high cancer potential state.

In some forms, a cytology assessment of ASC, a histology assessment of CIN-2, and a methylation site percentage for the set of 3 or more CpG dinucleotides of below 15.0% indicates a low cancer potential state, a cytology assessment of ASC, a histology assessment of CIN-2, and a methylation site percentage for the set of 3 or more CpG dinucleotides from 15.0% to 35.0% indicates a moderate cancer potential state, and a cytology assessment of ASC, a histology assessment of CIN-2, and a methylation site percentage for the set of 3 or more CpG dinucleotides of over 35.0% indicates a high cancer potential state.

In some forms, a cytology assessment of ASC, a histology assessment of CIN-3, and a methylation site percentage for the set of 3 or more CpG dinucleotides of below 35.0% indicates a moderate cancer potential state, and a cytology assessment of ASC, a histology assessment of CIN-3, and a methylation site percentage for the set of 3 or more CpG dinucleotides of over 35.0% indicates a high cancer potential state.

In some forms, a cytology assessment of HSIL, a histology assessment of CIN-1, and a methylation site percentage for the set of 3 or more CpG dinucleotides of below 15.0% indicates a low cancer potential state, a cytology assessment of HSIL, a histology assessment of CIN-1, and a methylation site percentage for the set of 3 or more CpG dinucleotides from 15.0% to 35.0% indicates a moderate cancer potential state, and a cytology assessment of HSIL, a histology assessment of CIN-1, and a methylation site percentage for the set of 3 or more CpG dinucleotides of over 35.0% indicates a high cancer potential state,

In some forms, a cytology assessment of HSIL, a histology assessment of CIN-2, and a methylation site percentage for the set of 3 or more CpG dinucleotides of below 10.0% indicates a low cancer potential state, a cytology assessment of HSIL, a histology assessment of CIN-2, and a methylation site percentage for the set of 3 or more CpG dinucleotides from 10.0% to 35.0% indicates a moderate cancer potential state, and a cytology assessment of HSIL, a histology assessment of CIN-2, and a methylation site percentage for the set of 3 or more CpG dinucleotides of over 35.0% indicates a high cancer potential state,

In some forms, a cytology assessment of HSIL, a histology assessment of CIN-3, and a methylation site percentage for the set of 3 or more CpG dinucleotides of below 20.0% indicates a moderate cancer potential state, and a cytology assessment of HSIL, a histology assessment of CIN-3, and a methylation site percentage for the set of 3 or more CpG dinucleotides of over 20.0% indicates a high cancer potential state.

Similar identifications can be made using other measures of methylation. For example, Table 9 shows identifications based on average genome methylation frequency per CpG site.

TABLE 8
Assessment of Cancer Potential State Based on Methylation Site Percentage

| | | Histology | | | |
|---|---|---|---|---|---|
| | Cancer | | HPV-associated | HPV-associated | HPV-associated |
| | Potential | | intraepithelial | intraepithelial | intraepithelial |
| Cytology | State | No Histology | neoplasia grade 1 | neoplasia grade 2 | neoplasia grade 3 |
| No | Low | Below 20.0% | Below 20.0% | Below 15.0% | N.A. |
| Cytology | Moderate | 20.0% to 50.0% | 20.0% to 50.0% | 15.0% to 50.0% | Below 35.0% |
| | High | Over 50.0% | Over 50.0% | Over 50.0% | Over 35.0% |
| LSIL | Low | Below 20.0% | Below 20.0% | Below 15.0% | Below 10.0% |
| | Moderate | 20.0% to 50.0% | 20.0% to 50.0% | 15.0% to 50.0% | 10.0% to 35.0% |
| | High | Over 50.0% | Over 50.0% | Over 50.0% | Over 35.0% |
| ASC | Low | Below 15.0% | Below 15.0% | Below 15.0% | N.A. |
| | Moderate | 15.0% to 35.0% | 15.0% to 50.0% | 15.0% to 35.0% | Below 35.0% |
| | High | Over 35.0% | Over 50.0% | Over 35.0% | Over 35.0% |
| HSIL | Low | Below 10.0% | Below 15.0% | Below 10.0% | N.A. |
| | Moderate | 10.0% to 35.0% | 15.0% to 35.0% | 10.0% to 35.0% | Below 20.0% |
| | High | Over 35.0% | Over 35% | Over 35.0% | Over 20% |

TABLE 9
Assessment of Cancer Potential State Based on Average
Genome Methylation Frequency per CpG Site

| | | Histology | | | |
|---|---|---|---|---|---|
| | Cancer | | HPV-associated | HPV-associated | HPV-associated |
| | Potential | | intraepithelial | intraepithelial | intraepithelial |
| Cytology | State | No Histology | neoplasia grade 1 | neoplasia grade 2 | neoplasia grade 3 |
| No | Low | Below 20.0% | Below 20.0% | Below 15.0% | N.A. |
| Cytology | Moderate | 20.0% to 50.0% | 20.0% to 50.0% | 15.0% to 45.0% | Below 35.0% |
| | High | Over 50.0% | Over 50.0% | Over 45.0% | Over 35.0% |
| LSIL | Low | Below 20.0% | Below 20.0% | Below 15.0% | Below 10.0% |
| | Moderate | 20.0% to 50.0% | 20.0% to 50.0% | 15.0% to 45.0% | 10.0% to 35.0% |
| | High | Over 50.0% | Over 50.0% | Over 45.0% | Over 35.0% |
| ASC | Low | Below 15.0% | Below 15.0% | Below 15.0% | N.A. |
| | Moderate | 15.0% to 35.0% | 15.0% to 50.0% | 15.0% to 35.0% | Below 35.0% |
| | High | Over 35.0% | Over 50.0% | Over 35.0% | Over 35.0% |
| HSIL | Low | Below 10.0% | Below 15.0% | Below 10.0% | N.A. |
| | Moderate | 10.0% to 35.0% | 15.0% to 35.0% | 10.0% to 35.0% | Below 20.0% |
| | High | Over 35.0% | Over 35% | Over 35.0% | Over 20% |

### C. Actions Based on Identifications

The disclosed methods include the determination, identification, indication, correlation, diagnosis, prognosis, etc. (which can be referred to collectively as “identifications”) of subjects, diseases, conditions, states, etc. based on measurements, detections, comparisons, analyses, assays, screenings, etc. For example, disclosed are methods that identify the cancer potential state, risk of cancer progression, diagnosis, prognosis, etc. of subjects based on HPV methylation states and the identification of subjects having particular cancer potential state, risk of cancer progression, diagnosis, prognosis, etc. based on HPV methylation states. Such identifications are useful for many reasons. For example, and in particular, such identifications allow specific actions to be taken based on, and relevant to, the particular identification made. For example, diagnosis of a particular disease or condition in particular subjects (and the lack of diagnosis of that disease or condition in other subjects) has the very useful effect of identifying subjects that would benefit from treatment, actions, behaviors, etc. based on the diagnosis. For example, treatment for a particular disease or condition in subjects identified is significantly different from treatment of all subjects without making such an identification (or without regard to the identification). Subjects needing or that could benefit from the treatment will receive it and subjects that do not need or would not benefit from the treatment will not receive it.

Accordingly, also disclosed herein are methods comprising taking particular actions following and based on the disclosed identifications. For example, disclosed are methods comprising creating a record of an identification (in physical—such as paper, electronic, or other—form, for example). Thus, for example, creating a record of an identification based on the disclosed methods differs physically and tangibly from merely performing a measurement, detection, comparison, analysis, assay, screen, etc. Such a record is particularly substantial and significant in that it allows the identification to be fixed in a tangible form that can be, for example, communicated to others (such as those who could treat, monitor, follow-up, advise, etc. the subject based on the identification); retained for later use or review; used as data to assess sets of subjects, treatment efficacy, accuracy of identifications based on different measurements, detections, comparisons, analyses, assays, screenings, etc., and the like. For example, such uses of records of identifications can be made, for example, by the same individual or entity as, by a different individual or entity than, or a combination of the same individual or entity as and a different individual or entity than, the individual or entity that made the record of the identification. The disclosed methods of creating a record can be combined with any one or more other methods disclosed herein, and in particular, with any one or more steps of the disclosed methods of identification.

As another example, disclosed are methods comprising making one or more further identifications based on one or more other identifications. For example, particular treatments, monitorings, follow-ups, advice, etc. can be identified based on the other identification. For example, identification of subject as having a disease or condition with, for example, a high level of a particular component or characteristic can be further identified as a subject that could or should be treated with a therapy based on or directed to the high level component or characteristic. A record of such further identifications can be created (as described above, for example) and can be used in any suitable way. Such further identifications can be based, for example, directly on the other identifications, a record of such other identifications, or a combination. Such further identifications can be made, for example, by the same individual or entity as, by a different individual or entity than, or a combination of the same individual or entity as and a different individual or entity than, the individual or entity that made the other identifications. The disclosed methods of making a further identification can be combined with any one or more other methods disclosed herein, and in particular, with any one or more steps of the disclosed methods of identification.

As another example, disclosed are methods comprising treating, monitoring, following-up with, advising, etc. a subject identified in any of the disclosed methods. Also disclosed are methods comprising treating, monitoring, following-up with, advising, etc. a subject for which a record of an identification from any of the disclosed methods has been made. For example, particular treatments, monitorings, follow-ups, advice, etc. can be used based on an identification and/or based on a record of an identification. For example, a subject identified as having a disease or condition with, for example, a high level of a particular component or characteristic (and/or a subject for which a record has been made of such an identification) can be treated with a therapy based on or directed to the high level component or characteristic. Such treatments, monitorings, follow-ups, advice, etc. can be based, for example, directly on identifications, a record of such identifications, or a combination. Such treatments, monitorings, follow-ups, advice, etc. can be performed, for example, by the same individual or entity as, by a different individual or entity than, or a combination of the same individual or entity as and a different individual or entity than, the individual or entity that made the identifications and/or record of the identifications. The disclosed methods of treating, monitoring, following-up with, advising, etc. can be combined with any one or more other methods disclosed herein, and in particular, with any one or more steps of the disclosed methods of identification.

The disclosed measurements, detections, comparisons, analyses, assays, screenings, etc. can be used in other ways and for other purposes than those disclosed. For example, the disclosed measurements, detections, comparisons, analyses, assays, screenings, etc. can be used categorize populations of subjects, monitor historic trends in the measurements, collect epidemiological data, etc. Thus, the disclosed measurements, detections, comparisons, analyses, assays, screenings, etc. do not encompass all uses of such measurements, detections, comparisons, analyses, assays, screenings, etc.

### D. Monitoring and Testing

An important result of the discoveries and the methods disclosed herein is the more accurate assessments, diagnoses, and prognoses they make possible. For example, by more accurately identifying low risk patients from cell collection, the disclosed methods can reduce the number of patients subjected to biopsy and other more invasive testing when it is not likely to be needed. As another example, by more accurately identifying low risk patients from cell collection, the disclosed methods can increase the number of patients that could benefit from more frequent re-testing to be re-tested at shorter intervals.

### E. Treatment

Subjects can be treated based on or following identification of cancer potential state in the disclosed methods. Treatment can also follow additional testing and assessment of the subject. The choice to treat or not to treat, as well as the selection of the mode, manner, and/or timing of treatment can be based on the identification of cancer potential state. Any treatment identified or developed for treating subjects indentified with a cancer or a potential for cancer of the type identified can be used. Many such treatments are known.

By “treating” or “treatment” is meant the medical management of a patient with the intent to cure, ameliorate, stabilize, or prevent a disease, pathological condition, or disorder. These terms include active treatment, that is, treatment directed specifically toward the improvement of a disease, pathological condition, or disorder, and also include causal treatment, that is, treatment directed toward removal of the cause of the associated disease, pathological condition, or disorder. These terms can mean that the symptoms of the underlying disease are reduced, and/or that one or more of the underlying cellular, physiological, or biochemical causes or mechanisms causing the symptoms are reduced. It is understood that reduced, as used in this context, means relative to the state of the disease, including the molecular state of the disease, not just the physiological state of the disease. In certain situations a treatment can inadvertently cause harm. In addition, these terms include palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. These terms do not require that the treatment in fact be effective to produce any of the intended results. It is enough that the results are intended.

The term “in need of treatment” as used herein refers to a judgment made by a caregiver (e.g. physician, nurse, nurse practitioner, or individual in the case of humans; veterinarian in the case of animals, including non-human mammals) that a subject requires or will benefit from treatment. This judgment is made based on a variety of factors that are in the realm of a care giver's expertise, but that include the knowledge that the subject is ill, or will be ill, as the result of a condition that is treatable by the compounds of the invention.

### EXAMPLES

### A. Example 1

### Distinct HPV16 Methylomes in Cervical Cancer Cells at Different Stages of Premalignancy

This example describes the comprehensive mapping of all 113 methylation sites in the HPV16 genomes contained in patient samples of cervical cells and the discovery of methylation patterns of biologic significance. HPV16 is the most common HPV type in anogenital cancer. The analysis of thirteen HPV16-positive premalignant samples from women undergoing routine cervical cancer screening showed that each cervical sample had one of three distinct DNA methylation patterns. The patterns agreed well with the cervical diagnoses arrived at by pathologic examination. They are also consonant with what is known regarding the biology of HPV-associated malignant progression.

Human papillomavirus (HPV) gene expression is dramatically altered during cervical carcinogenesis. Because dysregulated genes frequently show abnormal patterns of DNA methylation, it was realized that comprehensive mapping of the HPV methylomes in cervical samples at different stages of progression would reveal patterns of clinical significance. To establish this, thirteen HPV16-positive samples were obtained from women undergoing routine cervical cancer screening. Complete methylation data were obtained for 98.7% of the HPV16 CpGs in all samples by bisulfite-sequencing. Most HPV16 CpGs were unmethylated or methylated in only one sample. The other CpGs were methylated at levels ranging from 11% to 100% of the HPV16 copies per sample. The results showed three major patterns and two variants of one pattern. The patterns showed minimal or no methylation (A), low level methylation in the E1 and E6 genes (B), and high level methylation at many CpGs in the E5/L2/L1 region (C). Generally, pattern A was associated with negative cytology, pattern B with low-grade lesions, and pattern C with high-grade lesions. The severity of the cervical lesions was then ranked by the HPV16 DNA methylation patterns and, independently, by the pathologic diagnoses. Statistical analysis of the two rating methods showed highly significant agreement. Thus, analysis of the HPV16 DNA methylomes in clinical samples of cervical cells led to the identification of distinct methylation patterns which have utility as biomarkers of neoplastic cervical progression.

Persistent high-risk human papillomavirus (HPV) infection is a necessary cause of virtually all cases of cervical cancer. Routine cytologic screening of Papanicolau-stained cervical cells has dramatically reduced the incidence of cervical cancer, but cytology is not an ideal screening tool due to its low sensitivity for high grade lesions (Spitzer, 2002). This is why screening is typically repeated annually and why more than 5% of cervical cytology results without an obvious high-grade lesion (those with “atypical squamous cells of undetermined significance”) require close follow-up. The high cost of cervical cancer prevention is due to the frequency of Pap testing, the large number of cytologic and histologic follow-ups, and the expense of treating high grade CIN. A new HPV prophylactic vaccine is expected to further reduce the incidence of cervical cancer, although not for several years. Cervical cancer will continue to develop in unvaccinated women, the 3% of American women already infected with cancer-associated HPVs (Dunne et al., 2007) and women infected with high-risk types of HPV not in the vaccine, which cause about 30% of cervical cancer. The importance of continued cervical screening cannot be overemphasized, because inadequate screening in the post-vaccination era could negatively impact on the control of cervical cancer (Goldhaber-Fiebert et al., 2008)). At the same time however the sensitivity of cervical cytology will decline (Goldhaber-Fiebert et al., 2008). This situation urgently called for the discovery of novel biomarkers of cervical oncogenesis (Kiviat, Hawes, and Feng, 2008).

The hallmark of carcinogenesis is deregulation of cellular gene expression. A major mechanism controlling gene expression is DNA methylation. DNA methylation is a nonmutational, heritable and reversible epigenetic process. It is critical for normal development and cellular differentiation (Ballestar and Esteller, 2008), including epithelial differentiation (Paradisi et al., 2008). While the basic mechanism of DNA methylation has been known since the 1980s, intensive efforts to understand its role in gene regulation are more recent. Many studies indicate that the absence of DNA methylation in a gene promoter allows full expression of a gene, while its presence is correlated with gene silencing (Esteller, 2002). These studies are only a start though, as the vast majority of CpGs occur outside promoters and have not been surveyed systematically.

To better understand the role of DNA methylation in carcinogenesis, there is great desire to compare normal vs. malignant cells for differences in DNA methylation. Unfortunately the most accurate method for mapping the methylation status of each CpG in a DNA sequence, bisulfite-sequencing, is not cost-effective for this endeavor due to the very large size of the human genome (Bernstein, Meissner, and Lander, 2007). In contrast, it was realized that bisulfite-sequencing is entirely sufficient for mapping the relatively tiny genomes of viruses. Viruses cause an estimated 15% of all human cancers (zur Hausen, 1991), and their study over the past thirty years has been integral to understanding molecular processes that regulate both normal and transformed cells (DiMaio and Miller, 2006).

Previous studies of HPV16 DNA methylation (Badal et al., 2003; Kalantari et al., 2004) showed that the viral long control region (LCR) and early region were relatively unmethylated in most cervical lesions. In contrast the late gene L1, encoding the major viral capsid protein, was methylated at several CpGs in cervical carcinoma cell lines and most cervical carcinoma tissues, but not in most asymptomatic infections. More variable results were reported for premalignant lesions although low- and high-grade cervical intraepithelial neoplasia were generally not analyzed individually (Badal et al., 2003; Kalantari et al., 2004). Similar results have been reported for HPV18 in cervical lesions (Badal et al., 2004; Kalantari et al., 2008a; Turan et al., 2006; Turan et al., 2007) and for HPV16 in anal intraepithelial neoplasias (Wiley et al., 2005), penile carcinomas (Kalantari et al., 2008b) and oral squamous cell carcinomas (Balderas-Loaeza et al., 2007). These studies established a trend for increasing HPV 16/18 DNA methylation, particularly in the L1 gene, with increasing lesion severity but they did not identify individual CpGs whose methylation status specifically correlated with the pathology. On the other hand, only a small region the HPV 16 or HPV 18 genome was previously mapped by bisulfite-sequencing (Badal et al., 2004; Kalantari et al., 2008a; Turan et al., 2006; Turan et al., 2007). It was realized that comprehensive mapping of all 113 sites of potential DNA methylation in the HPV16 genomes contained in patient samples of non-malignant cervical cells at different stages of progression would reveal patterns of diagnostic and prognostic significance.

This example describes the analysis of thirteen HPV16-positive non-malignant samples from women undergoing routine cervical cancer screening. HPV16 is the most common HPV type in anogenital cancer. The results show three principal HPV16 DNA methylation patterns, the heaviest of which has two variants. The patterns are consonant with what is known regarding the biology of HPV-associated malignant progression given that the DNA methylation observed represses HPV16 gene expression. Furthermore, the patterns show significant agreement with the cervical diagnoses arrived at by pathologic examination.

1. Results

i. Identification of HPV16-Positive Cervical Samples

To identify samples of cervical cells containing HPV16 DNA, 72 samples collected for routine cervical screening were evaluated by PCR using two primer pairs specific for different regions of the HPV16 genome. Thirteen samples that generated clear bands of the appropriate sizes on agarose gels were selected for further study.

ii. Bisulfite-Conversion and Primer Design

The most accurate method for determining the methylation status of every CpG in a DNA sequence is bisulfite-sequencing. In this method, sodium bisulfite converts all unmethylated cytosines (C) to uracils but leaves methylated Cs intact. PCR is then performed to amplify the bisulfite-treated DNA and convert the uracils to thymines (T). Finally the original status of each C in the PCR product is determined by DNA sequencing, which shows C if the original C was methylated (meCpG) or T if it was not (CpG). Preliminary studies assessed the completeness of bisulfite-conversion using two substrates. One was a plasmid containing the full-length genome of the W12 isolate of HPV16 (Flores et al., 1999), which was methylated in vitro and amplified with primers described below. The other was the Universal Methylated DNA Standard, amplified with its own primers. Both control assays demonstrated complete bisulfite-conversion. As bisulfite-converted DNA contains only adenine, thymine and guanosine, except at methylated cytosines, the HPV16 genome was bisulfite-converted (in silico) for primer design. Twenty primer pairs were designed to collectively amplify all 113 CpGs in the HPV16 genome (Table 3). PCR amplification conditions were then optimized for each primer pair using the molecular clone of the W12 isolate of HPV16 (Alazawi et al., 2002) after actual in vitro DNA methylation and bisulfite-conversion.

TABLE 3
PCR primers for amplification of bisulfite-converted HPV16 DNA

| | Forward | Reverse | Product |
|---|---|---|---|
| Pair | Primer^{a} | Primer^{a} | Length^{b} |
| 1 | 7835-7859 | 86-115 | 185 |
| 2 | 99-124 | 435-462 | 364 |
| 3 | 416-444 | 666-691 | 276 |
| 4 | 666-692 | 791-816 | 151 |
| 5 | 822-849 | 1262-1288 | 467 |
| 6 | 1281-1310 | 1568-1595 | 315 |
| 7 | 1774-1800 | 2243-2272 | 499 |
| 8 | 2624-2648 | 3009-3026 | 403 |
| 9 | 3006-3035 | 3372-3398 | 393 |
| 10 | 3386-3411 | 3703-3725 | 340 |
| 11 | 3719-3748 | 3942-3966 | 248 |
| 12 | 3991-4015 | 4278-4307 | 317 |
| 13 | 4326-4355 | 4538-4563 | 238 |
| 14 | 4804-4833 | 5139-5168 | 365 |
| 15 | 5066-5095 | 5420-5449 | 384 |
| 16 | 5556-5585 | 5962-5986 | 431 |
| 17 | 6329-6358 | 6601-6627 | 299 |
| 18 | 6605-6632 | 7048-7077 | 473 |
| 19 | 7061-7088 | 7460-7484 | 424 |
| 20 | 7465-7492 | 7749-7778 | 314 |
| 12-1 | 7893-3917 | 4105-4140 | 248 |
| 12-2 | 4133-4162 | 4289-4319 | 187 |

^{a}Nucleotide number in the HPV16 genome.
^{b}Length in base-pairs.

iii. PCR Amplification

The DNAs from the thirteen cervical samples were bisulfite-converted and amplified with each of the first twenty primer pairs (Table 3). This work used further optimization of the PCR conditions for some products and some samples. Ultimately, all PCR products were amplified from all samples, indicating that each sample contained all parts of the HPV16 genome. The data suggest the presence of episomal HPV16 DNA but do not exclude the possibility of integrated HPV16 DNA in the form of concatamers or partially deleted/rearranged genomes, alone or in combination with episomal copies. The assessment of methylation state does not depend on integration or not of HPV. Integrated HPV16 DNA is found in about 6% of CIN-2 lesions and 19% of CIN-3 lesions, while low-grade lesions contain only episomal viral genomes (Vinokurova et al., 2008).

iv. Complete and Missing Data

To determine which HPV16 CpGs were methylated, the PCR products from each cervical sample were sequenced in both directions. In some cases, the DNA sequences were not fully readable. In those cases and others, the relevant DNA samples were reamplified and again sequenced in both directions. The data showed that the HPV16 sequences in all samples were essentially identical to the W12 variant (Flores et al., 1999) and that virtually all cytosines that were not part of CpGs had been converted to thymines, i.e. methylated CpAs, CpTs and CpGs were very rare.

Some data were initially missing, most frequently in PCR product 12 (Table 4). The difficulty obtaining readable sequence from product 12 was most likely due to secondary structure resulting from an extraordinarily high concentration of A+T nucleotides, the frequency of which would range from 86 to 88%, depending the methylation frequency. In order to obtain additional data, two new primer pairs were designed that together amplified a region containing the sequence of product 12 plus additional upstream and downstream sequences (primers 12-1 and 12-2, Table 3). DNA sequencing of the new PCR products provided new data for 12 CpGs with previously missing data. A subset of samples still had partially missing data at eight CpGs (Table 4).

TABLE 4
HPV16 CpGs with partially missing data

| | |
|---|---|
| No. of | DNA methylation in samples |
| samples with | with complete data |

| ORF | CpG^{a} | missing data | Frequency^{b} | Level^{c} |
|---|---|---|---|---|
| E1 | 1509 | 1/13 | 2/12 | 6.3 ± 4.5 |
| E1 | 1559 | 1/13 | 0/12 | N/A |
| E1 | 1566 | 2/13 | 1/11 | 4.5 ± 4.5 |
| E5 | 4016 | 2/13 | 0/11 | N/A |
| L2 | 4247 | 1/13 | 3/12 | 8.2 ± 4.4 |
| L2 | 4259 | 1/13 | 0/12 | N/A |
| L2 | 4268 | 5/13 | 0/8 | N/A |
| L2 | 4275 | 6/13 | 0/7 | N/A |

^{a}Position of the cytosine.
^{b}Number of samples with methylation/number of samples with data.
^{c}Percent of HPV16 copies with methylated CpG (mean ± S.E.M.).

Among the samples with complete data, five of the eight CpGs were unmethylated and three were methylated at low levels, ranging from 4.5 to 8.2% of the HPV16 copies. All together, complete methylation data were obtained for 1450 of the 1469 CpGs in the thirteen samples (98.7%).

v. Distribution and Methylation Frequency of all HPV16 CpGs

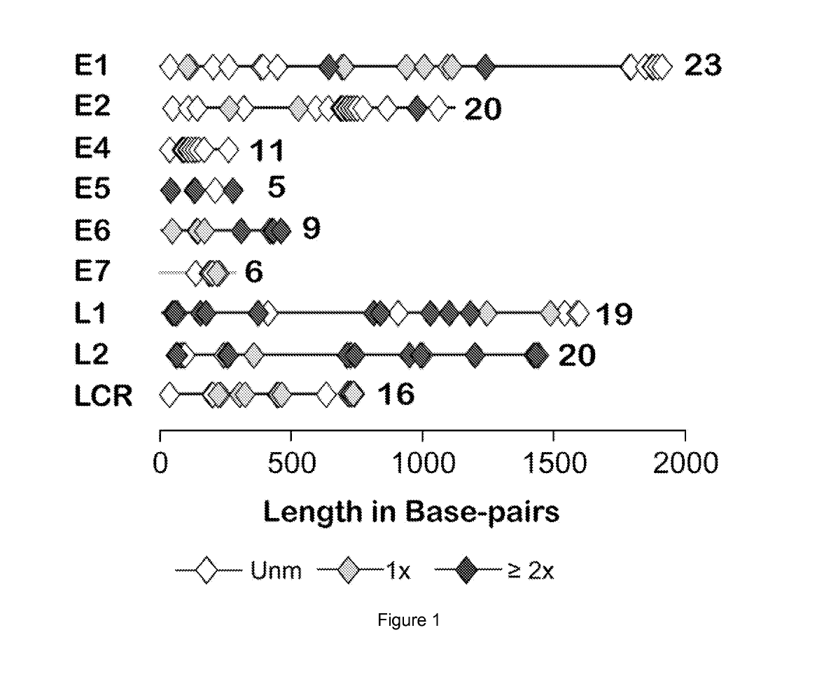

The HPV16 genome contains 113 CpGs, and multiple CpGs occur in each gene as well as the LCR. Direct sequencing of the individual PCR products frequently showed the presence of C and T in different reactions and/or C in one sequencing direction and T in the other. The variation was probably due to the heterogeneity of the population of HPV16 molecules in the original sample, and hence PCR product, and not to hemimethylation. This interpretation was also supported by peaks that contained both C and T in some chromatograms. To estimate the frequencies of methylation among the HPV16 copies per sample, the mean frequencies per sample were averaged, calculated by averaging all the sequence data (2.7±0.1 readable sequences per CpG per sample (mean±S.E.M.)). The frequency of methylation at the individual meCpGs ranged from a mean of 11.1% (at four CpGs with nine data points) to 100% of the HPV16 genomes per sample. Fifty-two CpGs, including all in the E4 ORF, all but one in the E7 ORF, and most in the E2 ORF were not methylated in any sample (FIG. 1). Thirty-two CpGs were methylated in just one sample, and only 29 were methylated in multiple samples. FIG. 5 shows the position of each CpG (diamond) in the context of the full-length gene or LCR. The numbers designate the start and end of an open reading frame or LCR.

vi. HPV16 DNA Methylation Patterns in Clinical Samples

The profiles of the HPV16 methylomes within each sample were also examined. As shown in FIG. 2, most methylated CpGs were heterogeneously methylated, as indicated by levels greater than 0 and less than 100% and by the error bars. From visual inspection of the individual profiles three patterns were deduced (FIG. 2). The first six samples were completely or nearly completely unmethylated (pattern A). The next two were methylated at limited numbers of CpGs, primarily in the early region (pattern B). The last five were heavily methylated at several CpGs, primarily in the late region (pattern C). Overall, the number of CpGs methylated per sample was 1.3±0.4 in pattern A, 9.0±1.0 in pattern B, and 22.2±3 in pattern C (mean±S.E.M.). While some meCpGs occurred in only one sample per pattern and therefore were not part of the pattern per se, the different levels indicate different degrees of susceptibility to DNA methylation (or demethylation) at different stages of disease.

vii. Cluster Analysis: Identification of Related Subgroups of HPV16 DNA Methylation Profiles

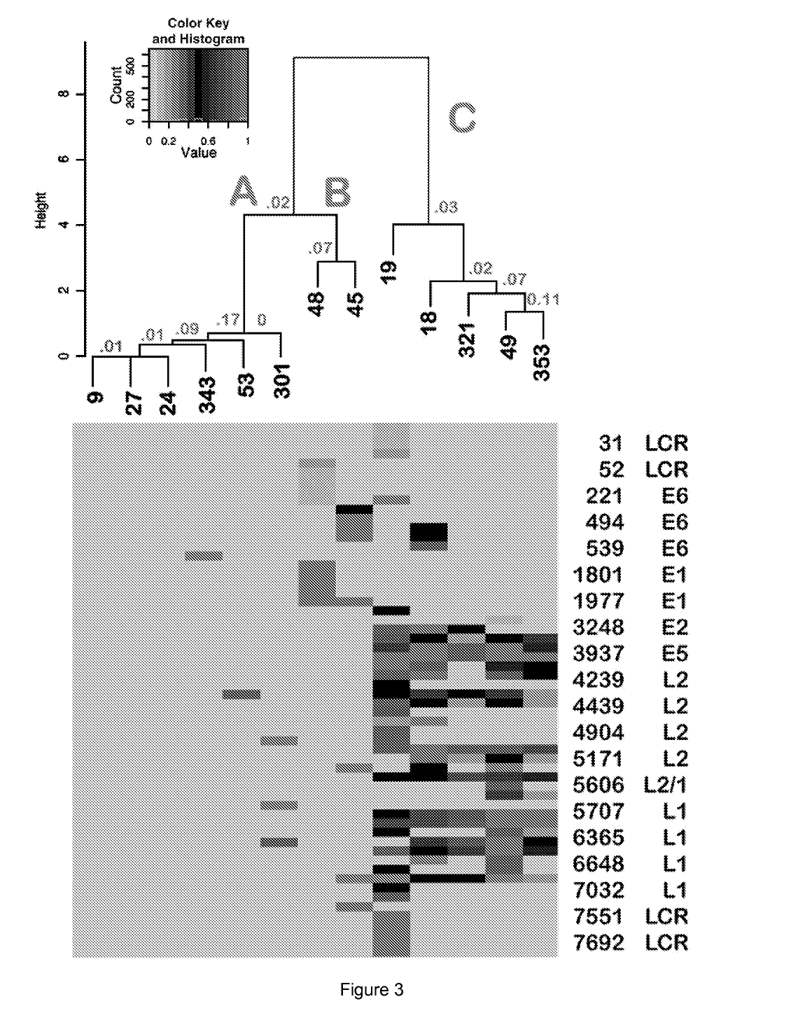

To further examine the relationships among the thirteen cervical samples, the data were subjected to cluster analysis. As shown in FIG. 3, the samples with patterns A and B formed one major branch with two closely related subgroups, one consisting of the six samples with pattern A, and the other, the two samples with pattern B. The second major branch contained the five samples with pattern C. As the probabilities of the clusters occurring by chance were small (FIG. 3), the visual impressions were validated.

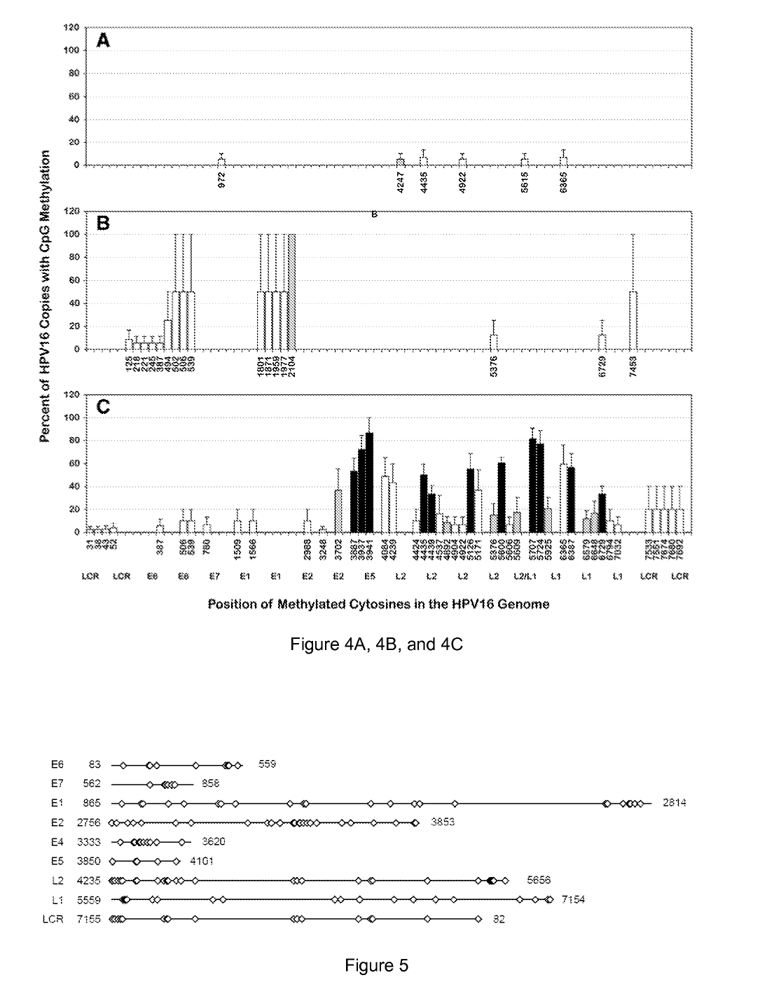

The mean data for each pattern was plotted to relate its major features to the HPV16 genome. CpGs that were methylated in multiple samples (potentially part of a pattern) vs. only one were distinguished. In pattern A, up to three CpGs were methylated, at low frequency, and they were located variously in the E1, L2 or L1 ORFs (FIG. 4A). Only one CpG in pattern A was methylated in two samples (in the L2 gene) and none in more than two. In pattern B, both samples were methylated at four or five contiguous CpGs in the E6 open reading frame (ORF) (from position 125 to 387 in sample #45, or position 494 to 539 in sample #48) (FIG. 4B). One (sample #45) also was methylated in the LCR at the CpG in E2 binding site 4 (E2BS#4). In pattern C, all five samples were heavily methylated at eleven CpGs located in the E5/L2/L1 region (black bars in FIG. 4C; Table 7), nine of which were pattern C-specific, i.e. not methylated in any other sample (3887, 3937, 3941, 4439, 5126, 5600, 5707, 5724, and 6387; Table 1; Table 7). The nine pattern C-specific CpGs included three of five in the E5 ORF, three of twenty in the L2 gene and four of nineteen in the L1 gene (one overlapping the L2 gene) (FIG. 4C) (for positions see Table 1). In summary, pattern A was characterized by minimal methylation, pattern B by methylation in the E1 and E6 ORFs, and pattern C by heavy methylation at nine specific CpGs located at the end of the early region (E5) and in the L2 and L1 ORFs but not including the L1 terminus.

viii. Correlation of Pathology with HPV16 DNA Methylation

The cervical cytology results and pathologic diagnoses were reviewed by an expert cytopathologist and two expert histopathologists. Five samples were diagnosed by cytology alone: two as negative, two as atypical squamous cells of undetermined significance (ASC-US, an equivocal diagnosis), and one as a low-grade squamous intraepithelial lesion (LSIL). Four samples with histologic follow-up were diagnosed as low-grade cervical intraepithelial neoplasia (CIN-1), and four others as high grade CIN (CIN-2/3). It was discovered that the high-grade lesions (CIN-2/3) generally have pattern C, which is consistent with DNA methylation silencing gene expression and the observation that heavily methylated HPV genes in pattern C are silenced during malignant progression. It was also discovered that the negative samples generally have pattern A, i.e. are the most different from pattern C, and that the CIN-1 lesions, being intermediate in severity, have an intermediate but functionally distinct level of methylation, i.e. pattern B. Three samples with ASC-US or LSIL and no follow-up had insufficient data for definitive diagnosis. Of the other samples, two with pattern A had negative cytology, both samples with pattern B showed signs of HPV infection and cervical intraepithelial neoplasia grade 1 (CIN-1), i.e. low-grade lesions, and three with pattern C were high-grade lesions (CIN-2/3) (FIG. 4). Thus seven of the ten cases with definitive diagnoses had the discovered correlation.

One apparently discordant low-grade lesion with pattern C (#19) was found upon re-review of the bisulfite sequencing data to be methylated at 15 CpGs that were not methylated in any other sample (Table 1). It was also completely unmethylated at two CpGs that were methylated in every other pattern C sample (Table 1). Re-review of the cluster analysis reinforced the importance of the differentially methylated CpGs because it showed that sample #19 was more distantly related to the other pattern C samples than they were to each other, by a factor of approximately two (FIG. 3). Together, the data led to the conclusion that sample #19 had a distinct variant of pattern C. Pattern C was therefore divided into a C-2 variant represented by sample #19 and a C-1 variant

TABLE 1
Methylated CpGs specific to pattern
C and unique to the C-2 variant

| | | |
|---|---|---|
| | | Methylation |
| Feature Location | Cytosine ^{a} | Frequency ^{b} |

Specific to pattern C (variants C-1 and C-2) ^{c}

| | | |
|---|---|---|
| E5 | 3887 | 53.3 ± 11.1 |
| E5 | 3937 | 72.0 ± 12.7 |
| E5 | 3941 | 86.7 ± 13.3 |
| L2 | 4439 | 33.3 ± 7.5 |
| L2 | 5126 | 55.0 ± 13.3 |
| L2/L1 | 5600 | 60.3 ± 4.8 |
| L1 | 5707 | 81.7 ± 9.3 |
| L1 | 5724 | 76.7 ± 12.2 |
| L1 | 6387 | 56.7 ± 11.7 |

Unique to variant C-2 ^{d}

| | | |
|---|---|---|
| Enhancer | 7533 | 100.0 |
| Enhancer | 7551 | 100.0 |
| Enhancer | 7674 | 100.0 |
| Enhancer ^{e} | 7680 | 100.0 |
| Enhancer | 7692 | 100.0 |
| Promoter | 31 | 12.5 |
| Promoter ^{e} | 38 | 12.5 |
| Promoter | 43 | 14.3 |
| Promoter ^{e} | 52 | 20.0 |
| E2 | 2988 | 50.0 |
| L2 | 4424 | 50.0 |
| L2 | 4537 | 80.0 |
| L2 | 4904 | 33.3 |
| L2 | 5171 | 0.0 ^{f} |
| L1 | 6365 | 0.0 ^{g} |
| L1 | 6794 | 50.0 |
| L1 | 7032 | 33.3 |

^{a} Position of the cytosine in the HPV16 genome.
^{b} Percent of HPV16 genomes per sample with methylation (mean ± S.E.M.).
^{c} Each CpG was sequenced 3.9 ± 0.3 times per sample (mean ± S.E.M.).
^{d} Each CpG was sequenced 4.7 ± 0.4 times (mean S.E.M.) in sample #19, with variant pattern C-2.
^{e} Cytosine located within an HPV16 E2 protein-binding site (E2BS).
^{f} Methylated in all pattern C samples except #19 at 45.8% ± 19.7% of the HPV16 copies per sample (mean ± S.E.M.)
^{g} Methylated in all pattern C samples except #19 at 74.2% ± 10.6% of the HPV16 copies per sample (mean ± S.E.M.)

represented by the other samples. The uniquely methylated CpGs in pattern C-2 (sample #19) included nine in the LCR. Five of six in the keratinocyte-specific enhancer were completely methylated and four of the five in the E6/E7 promoter were lightly methylated (Table 1). Since DNA methylation in the 5′ regulatory regions of genes, including the HPV16 LCR (Kim et al., 2003), usually represses expression of the gene(s) it controls, E6/E7 expression likely was compromised in sample #19. The reference to cytosine 4424 in Table 1 actually refers to the cytosine at position 4425 in the HPV16 genome.

The remaining two cases in which the HPV16 DNA methylation pattern did not agree with the pathologic diagnoses showed no meaningful differences in HPV16 methylation. One sample (#343) clearly had HPV16 DNA methylation pattern A but

TABLE 2
Lesion severity ranked by HPV16 DNA
methylation pattern vs. pathologicg

| | | | Pathologic diagnosis | | |
|---|---|---|---|---|---|
| | HPV16 DNA | | Neg. ^{a} | CIN1 | CIN2/3 |
| Pap | methylation | | Rank | | |
| result | pattern | Rank | 1 | 2 | 3 |
| Neg. ^{a} | A | 1 | #27 ^{f} | | |
| Neg. | A | 1 | #53 | | |
| HSIL ^{b} | A | 1 | | | #343 |
| ASC-US ^{c} | B | 2 | | #48 | |
| ASC-H ^{d} | B | 2 | | #45 | |
| LSIL ^{e} | C-2 | 2 | | #19 | |
| ASC-US | C-1 | 3 | | #353 | |
| ASC-US | C-1 | 3 | | | #18 |
| ASC-US | C-1 | 3 | | | #49 |
| ASC-US | C-1 | 3 | | | #321 |

^{a} Cytologically negative for intraepithelial lesion or malignancy.
^{b} High-grade squamous intraepithelial lesion.
^{c} Atypical squamous cells of undetermined significance.
^{d} ASC-US cannot rule out HSIL.
^{e} Low-grade squamous intraepithelial lesion (diagnosed as ASC-US before expert review).
^{f} Number of the cervical sample.

showed a high grade lesion by both cytology and histology. The other one (sample #353) had the high-risk variant of pattern C but was diagnosed as CIN-1. The early region of HPV16 in sample #353 was completely devoid of methylation, while the same region of the other pattern C samples, including sample #19, had one to four methylated CpGs. This tiny difference was however insufficient to consider the pattern of sample #353 as a variant. Finally, the extent of agreement between the HPV16 DNA methylation patterns and the pathologic diagnoses was evaluated after classifying pattern C-2 as a low-grade variant and excluding the three samples without definitive diagnoses (Table 2). Statistical analysis showed highly significant concordance between the two methods (P=0.005, Cohen's Kappa statistic).

2. Discussion

While it has been known for 25 years that papillomavirus genomes are highly methylated in carcinomas (Wettstein and Stevens, 1983), mapping the methylation status of specific sites began only recently. Previous studies of HPV16 DNA methylation mapped up to 19 CpGs at the 3′ end of the L1 ORF and the LCR (Badal et al., 2004; Kalantari et al., 2004). This example describes the precise mapping of 98.7% of all CpGs in the HPV16 genomes contained in thirteen cellular samples of cervical cells with pathologic diagnoses ranging from negative to CIN-3. This is the first comprehensive DNA methylation mapping for the entire genome of any virus. It is also the first to identify unique HPV16 DNA methylation marks that distinguish high-grade lesions from low-grade lesions and asymptomatic infections.

Most of the 113 CpGs in the HPV16 genome were unmethylated or methylated in only one sample. The methylated CpGs were located mainly in the bodies of HPV16 genes. CpG methylation within gene bodies has been previously reported, but little is known about the prevalence of such events or their biologic significance. Methylation within a gene body might repress expression of the gene in which it resides, another gene(s) via regulatory elements contained within the first gene, or merely reflect the stage of malignant progression in neoplastic cells.

Repeat sequencing of the HPV16 PCR products revealed heterogeneous methylation at most methylated CpGs, as previously reported for smaller genomic regions by molecular cloning (Kalantari et al., 2004; Turan et al., 2006; Turan et al., 2007). Despite the heterogeneity, the HPV16 methylomes in each sample showed one of three distinct patterns of HPV16 DNA methylation or a variant of the most highly methylated pattern. The existence of a limited number of patterns indicates that transfer and/or removal of methyl groups to CpGs in the HPV16 genome is not a random process, that cells with methylation (or not) at particular HPV16 CpGs have a selective growth advantage, and that the methylation of certain CpGs is incompatible with continued infection.

Pathologically, the samples with almost no HPV16 DNA methylation (pattern A) were the least severe, those with several methylated CpGs in the E1 and E6 ORFs were intermediate in severity (pattern B), and those with high frequency methylation, particularly in the E5/L2/L1 region (pattern C), the most severe. Excluding three samples with insufficient pathology, the HPV16 DNA methylation patterns ranked the severity of eight of ten lesions identically to the pathologic diagnoses. Moreover the agreement was statistically significant (P=0.005). Thus neoplastic progression was generally associated with increasing numbers of methylated CpGs and increasing proportions of methylated HPV16 molecules, in agreement with previous findings (Badal et al., 2004; Badal et al., 2003; Kalantari et al., 2004; Turan et al., 2006; Turan et al., 2007). The new discovery here identifies specific patterns of CpG methylation correlated with pathological state and nine specific CpGs in the E5 ORF, L2 ORF and 5′ two-thirds of the L1 gene that are highly methylated in high-grade lesions (Table 1).

High levels of HPV16 CpG methylation do not however necessarily indicate a high grade lesion, because the sample with by far the most methylation was a low-grade lesion with the C-2 variant pattern. In this lesion expression of the E6/E7 oncogenes were repressed due to the methylation of more than 80% of the CpGs in the enhancer/promoter region. Interestingly, the lesion spontaneously regressed as shown by follow-up cytology one year after biopsy. As the growth of transformed cervical cells relies on continued E6/E7 expression (DeFilippis et al., 2003), it was concluded that methylation of the HPV16 enhancer/promoter region was correlated with regression. It is worth noting in this context that complete methylation of the HPV16 promoter/enhancer region has previously been reported in a subset of asymptomatic infections (Badal et al., 2003) that might have been regressing.

The 3′ terminus of the L1 ORF was completely unmethylated in all samples. Previous bisulfite-sequencing studies have similarly reported the absence of methylation at the L1 terminus in most asymptomatic and low-grade cervical lesions as well as some high-grade cervical lesions (Kalantari et al., 2004; Turan et al., 2006; Turan et al., 2007). Other high-grade lesions however were highly methylated at the L1 terminus (Kalantari et al., 2004; Turan et al., 2006; Turan et al., 2007). Since DNA methylation and HPV gene expression both change dramatically during epithelial differentiation (Paradisi et al., 2008; Zheng and Baker, 2006), and the L1 terminus of episomal HPV16 transits from a hypermethylated state in undifferentiated cervical cells to a hypomethylated state upon the induction of differentiation (Kalantari et al., 2008a), the absence of methylation at the L1 terminus in at least some of our high grade lesions may reflect the absence of undifferentiated keratinocytes in cytology samples (this example) vs. tissues samples (previous studies).

There were two discordant cases. The low-grade lesion (CIN-1) with the C-1 methylation pattern was persistent as shown by follow-up cytology (LSIL) seven months after the biopsy (sample #353). This outcome indicates that the C-1 variant in a patient with CIN-1 indicates persistence. Persistent lesions are at greatly increased risk of malignant progression (Schiffman et al., 2005). The other case was a high-grade lesion (HSIL and CIN-2) with pattern A (#343). In this case it is that the high grade pathology was caused by co-infection with a high-risk HPV type(s) other than HPV16. Since concurrent HPV infections are common (Trottier et al., 2006), the evaluation of HPV co-infection and the assessment of additional HPV methylomes can be used for diagnostic and prognostic assessment of cervical disease.

In summary, HPV16 DNA methylation patterns can be used as biomarkers of cervical carcinogenesis.

3. Materials and Methods

i. Origin of Samples

Seventy-two residual samples of exfoliated cervical cells were obtained from patients being routinely screened for cervical cancer by the Department of Pathology at Yale University. The cervical cells were collected in either PreservCyt® solution and held at room temperature for approximately one month prior to use. The samples were obtained with approval from the Yale Human Investigation Committee.

ii. DNA Purification

High molecular weight DNAs were extracted from cervical samples using MasterPure™ DNA purification kits (EPICENTRE®Biotechnologies, Madison, Wis. 85201).

iii. HPV16 DNA Screening

Sample DNAs were screened for HPV16 DNA by PCR using two primer pairs that amplified fragments containing nucleotides (nts) 79 to 559 or nts 1800 to 1942. PCR reactions were performed using Taq PCR Master Mix Kits (Qiagen Inc., Valencia, Calif. 91355) with primers at 10 μm concentration. The PCR profile was: 94° C.×5 minutes, followed by 35 cycles of 94° C.×20 seconds, 55° C.×45 seconds×72° C. for 1 minute, with a final incubation at 72° C. for 10 minutes.

iv. Bisulfite Modification

Sample DNAs were modified using the DNA Methylation-Gold Kit™ (catalog number D5006, Zymo Research Corp., Orange, Calif. 92867) according to the manufacturer's instructions. To control for complete bisulfite-conversion, a plasmid containing the complete genome of the W12 isolate of HPV16 was methylated in vitro using the CpG Methyltransferase SssI (New England Biolabs, Ipswich, Mass.) and used as a substrate. Other control reactions used the Universal Methylated DNA Standard (catalog number D5010, (ZYMO Research, Orange, Calif. 92867).

v. Amplification of Bisulfite-Treated DNAs

Twenty-two pairs of primers were designed to amplify bisulfite-modified HPV16 DNA using the MethPrimer Design program (see web site urogene.org/methprimer/indexl.html) (Li). The primers are listed in Table 1 without M13 tails, which were added to facilitate DNA sequencing. The PCR amplification conditions for each primer set were optimized for MgCl2 concentration (1.5 to 4.0 μm) and annealing and elongation temperature (50° C. to 68° C.). Each PCR reaction contained 1.25 units of AmpliTaq Gold (catalog number 808-0241) (Roche Applied Science, Indianapolis, Ind. 46250) and 0.5 units of PfuTurbo® polymerase (Stratagene, La Jolla, Calif.). The standard optimized PCR profile was 95° C.×10 minutes, followed by five cycles of 95° C.×1 minute, 54° C. to 60° C.×2 minutes, 72° C.×3 minutes, and 35 cycles at 95° C.×1 minute, 60° C.×2 minutes, 72° C.×2 minutes, with a final incubation at 72° C. for 10 minutes. PCR reactions were performed in a MasterCycler® Gradient (Eppendorf Scientific Inc., Westbury, N.Y. 11590). The production of each PCR product was confirmed by electrophoresis in ethidium-bromide stained agarose gels. Further optimization was used to amplify some PCR products from several patient samples.

vi. DNA Sequencing

PCR products were purified and sequenced by Agencourt Bioscience Corporation (Beverly, Mass.), and the DNA sequencing data were analyzed using the multiple sequence alignment program Clustal W (see web site ebi.ac.uk/Tools/clustalw/).

vii. Statistical Analysis

Hierarchical clustering was performed using the hclust library in the R statistical package (Team, 2007) using Ward's minimum variance method with Euclidean distance metric. Average expression is shown with dark and black shagging, above-average expression in moderate shading, and below-average expression in light shading. The dendrograms were generated as defined for hierarchical clustering. Cluster stability was evaluated and permutation-based cluster stability P-values calculated using the multi-scale permutation clustering (R package ‘pvclust’ (Suzuki and Shimodaira, 2006)).

### B. Example 2

### Correlation of the Methylation State of 19 HPV16 Methylation Sites with Cervical Cancer and Risk of Cervical Cancer

1. Methods

Specimens of cervical cells for the first (N=13) and second (N=10) studies were obtained from the Cytology Service of the Department of Pathology at Yale, from women being routinely screened for cervical cancer by private physicians in the greater New Haven area. This population is primarily white and of middle or upper-middle socioeconomic status. For the third study, collaborators at the Fred Hutchinson Cancer Institute provided archived DNAs extracted from cervical specimens previously collected from African women who presented to community based clinics for non-cervical cancer related problems and had not been previously screened for cervical cancer (N=12). DNA methylation was analyzed by Sanger DNA bisulfite-sequencing as described in Example 1.

2. Results

i. Differential HPV16 CpG Methylation.

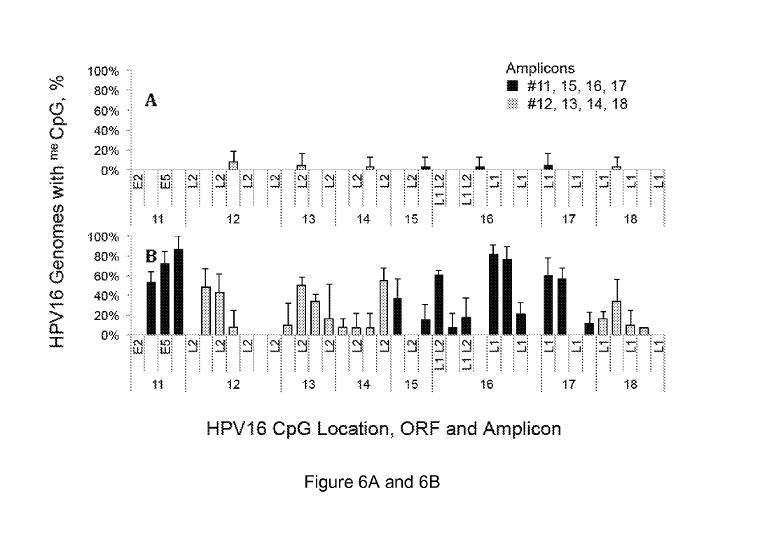

The results of the first study (Example 1) showed differential methylation in a region of the HPV16 genome containing the E5, L1 and L2 genes. In five of the thirteen cases, 18 of the 38 CpGs in this region were heavily methylated (18.4±1.7) vs. a single CpG in the other eight cases (1.0±0.4), a highly significant difference (P<1×10-7) (FIG. 6; Example 1). Further analysis by hierarchical clustering revealed two distinct branches, corresponding to the five or eight cases, respectively. Thus the cervical HPV16 infections were divided into two subgroups based differential methylation of CpGs in the E5/L2/L1 region. It was realized that analysis of fewer CpGs could provide good correlations since multiple HPV16 CpGs were differentially methylated in the first study. To demonstrate this, subsequent studies analyzed HPV16 DNA methylation in four PCR amplicons containing 19 of the 38 HPV16 CpGs (FIG. 6; Example 1). In all three studies, the majority of cases were either completely unmethylated or methylated at high frequency, while a minority were methylated at low frequency (Table 5). The difference in each study was highly significant (P<1.0×10-5, for each study). These results confirm the reproducibility of the first study (Example 1). The new results also extend the original findings by showing that differential HPV16 DNA methylation is not limited to socioeconomically advantaged white women being routinely screened in the United States (1st and 2nd studies) but also occurs in poor African women who were not screened previously (3rd study). The great disparities between these two populations of women indicate that differential HPV16 DNA methylation is a general feature of cervical infections.

TABLE 5
Three Studies Show Differentiation HPV16 Methylation

| Methylation Frequency | | |
|---|---|---|
| None | Low | High |

| Study | No.^{a} | % | No. | % | No. | % |
|---|---|---|---|---|---|---|
| 1 | 0/19 | 0 | 1/19 | 5 | 9/19 | 47 |
| | 0/19 | 0 | 2/29 | 11 | 10/19 | 53 |
| | 0/19 | 0 | | 0 | 11/19 | 58 |
| | 0/19 | 0 | | 0 | 11/19 | 58 |
| | 0/19 | 0 | | 0 | 15/19 | 79 |
| | 0/19 | 0 | | 0 | | 0 |
| 2 | 0/19 | 0 | 1/19 | 5 | 8/19 | 42 |
| | 0/19 | 0 | 4/19 | 21 | 11/19 | 58 |
| | 0/19 | 0 | | 0 | 16/19 | 84 |
| | 0/19 | 0 | | 0 | | 0 |
| | 0/19 | 0 | | 0 | | 0 |
| 3 | 0/19 | 0 | 3/18 | 17 | 13/19 | 72 |
| | 0/19 | 0 | 3/18 | 17 | 13/19 | 72 |
| | 0/18 | 0 | | 0 | 14/17 | 82 |
| | 0/19 | 0 | | 0 | 16/19 | 84 |
| | | | | 0 | 15/19 | 94 |
| | | | | 0 | 18/19 | 95 |

^{a}Number of meCpGs/number of CpGs with data, by case.

ii. Analysis of Individual CpGs.

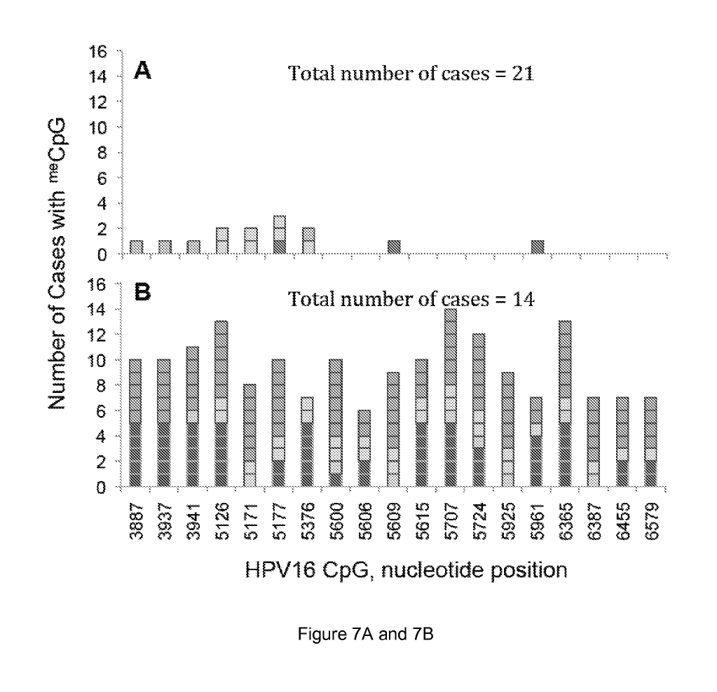

To establish that certain of the 19 CpGs were preferentially methylated, the number of cases with methylation at the individual CpGs was analyzed. The results show that every CpG was methylated in six to fourteen of the high frequency cases (42% to 100%) versus a maximum of three low frequency cases (14%) (FIG. 7). Since four of the 19 CpGs were unmethylated in the first study (Example 1), these data extend the original findings.

iii. Correlation Between CpG Methylation and Pathology.

The relationship between the methylation frequency and the cervical pathology was also assessed. Table 6 shows the frequencies of methylated CpGs, stratified by the pathology of the lesions. Among the cases with cytology only (no biopsy), the negative specimens were completely unmethylated and the low-grade LSIL lesions were minimally methylated, whereas the group of atypical ASCUS lesions was methylated at a frequency of 17% (Table 6). The substantially higher level of methylation among the ASCUS lesions correlates with their substantially greater likelihood of being diagnosed as high-grade CIN or cancer on biopsy. Among the cases with biopsy-confirmed diagnoses, the frequency of methylation increased progressively and dramatically with the severity of the pathology (Table 6). It was 1.6-fold higher in the CIN-2 vs. CIN-1 lesions, and 2.6-fold higher in the CIN-3 vs. CIN-2 lesions. In contrast, there was no difference between the invasive cervical cancers and the high-grade CIN-3 lesions.

TABLE 6
Correlation Between Methylation and Cervical Pathology

| | | | |
|---|---|---|---|
| | Risk of | | Methylation |
| Pathology | Progression^{a} | N^{b} | Frequency^{c} |

| | | | | |
|---|---|---|---|---|
| Cytology | Neg | None | 2 | 0 |
| | LSIL | Low | 6 | 1.8 ± 1.8 |
| | ASCUS | Equivocal | 5 | 16.8 ± 11.0 |
| Histology | CIN-1 | Low | 7 | 18.9 ± 9.7 |
| | CIN-2 | Moderate | 4 | 31.0 ± 11.2 |
| | CIN-3 | High | 2 | 81.6 ± 2.6 |
| | Cancer | — | 5 | 83.1 ± 4.9 |

^{a}Assessed by natural history studies based on pathology.
^{b}Number of cases per diagnosis.
^{c}Percent of 19 CpGs with methylation (mean ± SEM).

iv. Individual Patterns of HPV16 DNA Methylation.

To take full advantage of the DNA bisulfite sequencing data, the individual HPV16 DNA methylation profiles of the 35 cases were examined (Table 10). Nine specific CpGs were methylated in all cervical cancers (Table 10, open and shaded squares, □ ▪) and four other CpGs were methylated in four of the five cancers (the fifth lacked data) (Table 10, open and shaded circles, ∘ ). The same CpGs were also methylated in both high-grade CIN-3 lesions. The nearly identical profiles of the CIN-3 lesions and cervical cancers indicate that the CIN-3 lesions are actually invasive but that the invasive area was not biopsied or not properly interpreted on histology. Some CIN-1 and CIN-2/3 lesions were completely unmethylated, whereas others were methylated at various CpGs and at various levels (Table 10). This variability parallels the variability in clinical outcomes associated with CIN-1 and especially CIN-2 lesions. Thus, different HPV16 DNA methylation profiles correlate with different risks of malignant progression and the three unmethylated CIN-2/3 cases could have been assessed at minimal risk of short-term progression and could have been safely managed by conservative follow-up, sparing the patient unnecessary cervical surgery. Similarly, the CIN-1 case with relatively high level methylation indicates that this patient had a significantly elevated risk of malignant progression and could have best been managed by more frequent clinical follow-up than other CIN-1 lesions (case 7, Table 10). Finally, one unbiopsied case with “atypical” (ASCUS) cytology was highly methylated (case 5, Table 10), indicating that this patient may have had a high-grade cervical lesion lost to follow-up. Tables 8 and 9 assess risk of cancer progression (cancer potential state) based on combination of pathology assessments and methylation states.

Table 10 shows the methylation profile of 35 cases. The CpG number is listed in the top, middle, and bottom rows and refers to the CpG number indicated in Table 7. The numbers in the left column indicate the sample (patient) number. The percentage of genomes methylated at the indicated position is depicted by shading. No shading (□, ∘, ⋄) indicates 0% methylation; light shading ( [symbol] , [symbol] , [symbol] ) indicates 1-49% methylation; dark shading ( [symbol] , [symbol] , [symbol] ) indicates 50-99% methylation; black (▪, , ♦) indicates 100% methylation.

TABLE 10
Methylation Profiles of 35 Cases

| | 1 | 2 | 3 | 4 | 5 | 6 | 7 | 8 | 9 | 10 | 11 | 12 | 13 | 14 | 15 | 16 | 17 | 18 | 19 |
|---|---|---|---|---|---|---|---|---|---|---|---|---|---|---|---|---|---|---|---|
| | Negative | | | | | | | | | | | | | | | | | | |
| 1 | ◯ | ◯ | ◯ | □ | □ | □ | ⋄ | □ | ⋄ | □ | ⋄ | □ | □ | □ | ⋄ | ◯ | ⋄ | ◯ | ⋄ |
| 2 | ◯ | ◯ | ◯ | □ | □ | □ | ⋄ | □ | ⋄ | □ | ⋄ | □ | □ | □ | ⋄ | ◯ | ⋄ | ◯ | ⋄ |
| | LSIL | | | | | | | | | | | | | | | | | | |
| 1 | ◯ | ◯ | ◯ | □ | □ | □ | ⋄ | □ | ⋄ | □ | ⋄ | □ | □ | □ | ⋄ | □ | ⋄ | ◯ | ⋄ |
| 2 | ◯ | ◯ | ◯ | □ | □ | □ | ⋄ | □ | ⋄ | □ | ⋄ | □ | □ | □ | ⋄ | □ | ⋄ | ◯ | ⋄ |
| 3 | ◯ | ◯ | ◯ | □ | □ | □ | ⋄ | □ | ⋄ | □ | ⋄ | □ | □ | □ | ⋄ | □ | ⋄ | ◯ | ⋄ |
| 4 | ◯ | ◯ | ◯ | □ | □ | □ | ⋄ | □ | ⋄ | □ | ⋄ | □ | □ | □ | ⋄ | □ | ⋄ | ◯ | ⋄ |
| 5 | ◯ | ◯ | ◯ | □ | □ | □ | ⋄ | □ | ⋄ | □ | ⋄ | □ | □ | □ | ⋄ | □ | ⋄ | ◯ | ⋄ |
| 6 | ◯ | ◯ | ◯ | □ | □ | □ | ⋄ | □ | ⋄ | [symbol] | ⋄ | □ | □ | □ | [symbol] | □ | ⋄ | ◯ | ⋄ |
| | ACSUS | | | | | | | | | | | | | | | | | | |
| 1 | ◯ | ◯ | ◯ | □ | □ | □ | ⋄ | □ | ⋄ | □ | ⋄ | □ | □ | □ | ⋄ | □ | ⋄ | ◯ | ⋄ |
| 2 | ◯ | ◯ | ◯ | □ | □ | □ | ⋄ | □ | ⋄ | □ | ⋄ | □ | □ | □ | ⋄ | □ | ⋄ | ◯ | ⋄ |
| 3 | ◯ | ◯ | ◯ | □ | □ | □ | [symbol] | □ | ⋄ | □ | ⋄ | □ | □ | □ | ⋄ | □ | ⋄ | ◯ | ⋄ |
| 4 | [symbol] | [symbol] | [symbol] | □ | □ | □ | [symbol] | □ | ⋄ | □ | ⋄ | □ | □ | □ | ⋄ | □ | ⋄ | ◯ | ⋄ |
| 5 | ◯ | ◯ | ◯ | [symbol] | ▪ | [symbol] | [symbol] | [symbol] | [symbol] | [symbol] | ♦ | ▪ | ▪ | [symbol] | ⋄ | □ | ⋄ | ◯ | ⋄ |
| | 1 | 2 | 3 | 4 | 5 | 6 | 7 | 8 | 9 | 10 | 11 | 12 | 13 | 14 | 15 | 16 | 17 | 18 | 19 |
| | CIN-1 | | | | | | | | | | | | | | | | | | |
| 1 | ◯ | ◯ | ◯ | □ | □ | □ | ⋄ | □ | ⋄ | □ | ⋄ | □ | □ | □ | ⋄ | □ | ⋄ | ◯ | ⋄ |
| 2 | ◯ | ◯ | ◯ | □ | □ | □ | ⋄ | □ | ⋄ | □ | ⋄ | □ | □ | □ | ⋄ | □ | ⋄ | ◯ | ⋄ |
| 3 | ◯ | ◯ | ◯ | □ | □ | □ | ⋄ | □ | ⋄ | □ | ⋄ | □ | □ | □ | ⋄ | □ | ⋄ | ◯ | ⋄ |
| 4 | ◯ | ◯ | ◯ | □ | □ | [symbol] | ⋄ | □ | ⋄ | □ | ⋄ | □ | □ | □ | ⋄ | □ | ⋄ | ◯ | ⋄ |
| 5 | ◯ | ◯ | ◯ | ▪ | ▪ | ▪ | | □ | ⋄ | □ | ⋄ | □ | □ | □ | ⋄ | □ | ⋄ | ◯ | ⋄ |
| 6 | | | | | □ | □ | | □ | ⋄ | □ | | | | □ | ⋄ | [symbol] | ⋄ | ◯ | |
| 7 | | | | [symbol] | □ | □ | | □ | [symbol] | □ | ♦ | ▪ | [symbol] | □ | | | ⋄ | ◯ | ⋄ |
| | CIN-2 or CIN-2/3 | | | | | | | | | | | | | | | | | | |
| 1 | ◯ | ◯ | ◯ | □ | □ | □ | ⋄ | □ | ⋄ | □ | ⋄ | □ | □ | □ | ⋄ | □ | ⋄ | ◯ | ⋄ |
| 2 | ◯ | ◯ | ◯ | □ | □ | □ | | □ | ⋄ | □ | ⋄ | □ | □ | □ | ⋄ | □ | ⋄ | ◯ | ⋄ |
| 3 | ◯ | ◯ | ◯ | □ | □ | □ | ⋄ | □ | ⋄ | □ | ⋄ | □ | □ | □ | ⋄ | □ | ⋄ | ◯ | ⋄ |
| 4 | ◯ | ◯ | ◯ | ▪ | ▪ | [symbol] | | □ | ⋄ | □ | ⋄ | □ | □ | □ | ⋄ | □ | ⋄ | ◯ | ⋄ |
| 5 | [symbol] | [symbol] | [symbol] | [symbol] | □ | □ | [symbol] | □ | ⋄ | □ | [symbol] | [symbol] | □ | □ | [symbol] | [symbol] | ⋄ | ◯ | ⋄ |
| 6 | ◯ | ◯ | [symbol] | □ | □ | □ | ⋄ | ▪ | ⋄ | [symbol] | ⋄ | ▪ | ▪ | [symbol] | ⋄ | [symbol] | ⋄ | [symbol] | ⋄ |
| 7 | [symbol] | | | ▪ | □ | [symbol] | [symbol] | □ | ⋄ | □ | [symbol] | [symbol] | □ | □ | [symbol] | [symbol] | [symbol] | ◯ | ⋄ |
| 8 | [symbol] | | | [symbol] | □ | [symbol] | [symbol] | [symbol] | [symbol] | □ | ♦ | ▪ | [symbol] | □ | ♦ | ▪ | [symbol] | ◯ | [symbol] |
| | CIN-3 | | | | | | | | | | | | | | | | | | |
| 1 | ◯ | ◯ | ◯ | [symbol] | ▪ | ▪ | ♦ | ▪ | ♦ | ▪ | ♦ | ▪ | ▪ | [symbol] | [symbol] | [symbol] | [symbol] | | [symbol] |
| 2 | | | | ▪ | ▪ | ▪ | ⋄ | ▪ | ⋄ | ▪ | [symbol] | ▪ | ▪ | ▪ | ⋄ | ▪ | ♦ | | ♦ |
| | Invasive Carcinoma | | | | | | | | | | | | | | | | | | |
| 1 | | | | [symbol] | [symbol] | [symbol] | | ▪ | ⋄ | ▪ | ⋄ | ▪ | ▪ | [symbol] | ⋄ | ▪ | ⋄ | | ⋄ |
| 2 | | | | ▪ | ▪ | ▪ | | ▪ | ⋄ | ▪ | ⋄ | ▪ | ▪ | [symbol] | ⋄ | [symbol] | ⋄ | [symbol] | ⋄ |
| 3 | | | | ▪ | ▪ | ▪ | | ▪ | ⋄ | ▪ | ⋄ | ▪ | ▪ | ▪ | ⋄ | ▪ | [symbol] | | ♦ |
| 4 | | | | ▪ | ▪ | ▪ | ⋄ | ▪ | ♦ | ▪ | ♦ | ▪ | ▪ | ▪ | ♦ | ▪ | ♦ | | ♦ |
| 5 | | | | ▪ | ▪ | ▪ | ⋄ | ▪ | ♦ | ▪ | ♦ | ▪ | ▪ | ▪ | ♦ | ▪ | ♦ | | ♦ |
| | 1 | 2 | 3 | 4 | 5 | 6 | 7 | 8 | 9 | 10 | 11 | 12 | 13 | 14 | 15 | 16 | 17 | 18 | 19 |

v. Study 1

Of the 19 selected CpGs in the E5/L2/L1 ORFs, 2.0%±4.0% were methylated in eight cases, which can be compared to 61.1%±12.1% in the other five cases (mean±1 standard deviation (SD)). This difference was highly significant (P=0.00023, unpaired two-tailed t-test with unequal variance). The mean minus two SDs for the heavily methylated subset of cases is 36.8%. Using 37% as the cut-off value for significant cervical pathology (our rule), the only high-grade CIN-3 lesion (100% sensitivity) and two of the three moderate-grade CIN-2 lesions (67% sensitivity) were detected. The third CIN-2 lesion, which had 0% methylation, would be considered at minimal risk of short-term malignant progression.

TABLE 11
Study 1 Results for 19 CpGs in the E5/L2/L1 region of HPV16

| Sub- | | HPV16 Met | | Methylation Frequency ^{c} | |
|---|---|---|---|---|---|
| group | Case | Pattern ^{a} | Diagnosis ^{b} | by case | by subgroup |
| 1 | 1 | A | Neg | 0 | 2.0 ± 4.0 |
| | 2 | A | Neg | 0 | |
| | 3 | A | ASC-H | 0 | |
| | 4 | A | ASC | 0 | |
| | 5 | A | CIN-1 | 0 | |
| | 6 | A | CIN-2 | 0 | |
| | 7 | B | CIN-1 | 5 | |
| | 8 | B | LSIL | 11 | |
| 2 | 9 | C | CIN-1 | 47 | 61.1 ± 12.1 |
| | 10 | C | CIN-2 | 53 | |
| | 11 | C | CIN-1 | 63 | |
| | 12 | C | CIN-2 | 63 | |
| | 13 | C | CIN-3 | 79 | |

^{a} The patterns reflected minimal (A or B) or heavy (C) methylation in the E5/L2/L1 region.
^{b} The histologic diagnosis if biopsied or the cytologic interpretation.
^{c} Methylation of 19 CpGs in the E5/L2/L1 region (percent).

VI. Study 2

In study 2, ten new HPV16 DNA-positive cervical cytology samples were analyzed for methylation at the 19 HPV16 CpGs. The results showed methylation frequencies of 3.8%±7.9% in seven cases and 61.4%±21.3% in three cases (mean±SD). Using the 37% rule, both moderate/high-grade lesions were detected (one CIN-2, one CIN-3). Thus study 2 confirmed the results of study 1.

vii. Study 3

Study 3 was performed in collaboration with investigators at another institution who provided the specimens. The specimens had been collected in previous studies of HPV and cervical cancer and archived. The population of women in study 3 was entirely different from the population in studies 1 and 2 (Table 12).

TABLE 12
Sources of Cervical Cytology Specimens

| | | | | | | Cervical cancer | | |
|---|---|---|---|---|---|---|---|---|
| | | | | | Socio-economic | Screening | Health Care | Geographic |
| Study | Specimen | N | Diagnoses | Ethnicity | Status | history | Provider | Location |
| 1, 2 | Recent | 13, | Premalignant | Mostly | Middle to upper | Routinely | Private MD | New |
| | | 10 | | white | | screened | | Haven |
| 3 | Archived | 12 | Premalig. & | African | Disadvantaged | Not screened | Community- | Senegal |
| | | | malignant | (black) | | before | based | |

The methylation status of the 19 HPV16 CpGs was analyzed using coded specimens, i.e. blinded. The results identified six of the 12 cases with methylation frequencies of >37%. After breaking the code, these cases were found to include 5 of 5 invasive cervical cancers and one of two CIN-3 lesions. Given that the study population was entirely different in study 3, the results show that differential HPV16 DNA methylation is a general characteristic of cervical HPV16 infections. The results further validated the 37% rule. They also extended our prior results by demonstrating that the 19 CpGs which were selected on the basis of high frequency methylation in CIN-2/3 lesions were also methylated at high frequency in all invasive cervical cancers.

viii. Correlation of HPV16 DNA Methylation Frequency with Cervical Diagnoses

The frequency of HPV16 DNA methylation at the 19 CpGs increases progressively and dramatically as the severity of the pathology increases. As shown in Table 13, the percentage of cases with >37% CpG methylation is significantly greater among lesions diagnosed as CIN-2 or worse vs. CIN-1 or less (P=0.001, Fisher's Exact Test). The actual CpG methylation frequency is also significantly greater for cases diagnosed as CIN-2, CIN-3 or ICC vs. CIN-1 or negative (P=0.002, t-test with unequal variance).

TABLE 13
HPV16 DNA methylation results stratified
by the pathologic diagnoses

| | HPV16 DNA Methylation | | |
|---|---|---|---|
| | Cases ^{a} | | CpGs ^{c} |
| Dx | N | % ^{b} | Mean % ± SEM |
| Neg | 2 | 0 | 0 |
| CIN-1 | 7 | 29 | 19 ± 10 |
| CIN-2 | 6 | 50 | 29 ± 11 |
| CIN-3 | 4 | 75 | 62 ± 21 |
| Cancer | 5 | 100 | 83 ± 5 |

^{a} Cases with negative cytology or a biopsy-confirmed diagnosis from the three studies.
^{b} Percent with >37% methylation.
^{c} The 19 selected CpGs.

ix. Prognostic Potential of HPV16 DNA Methylation Analysis

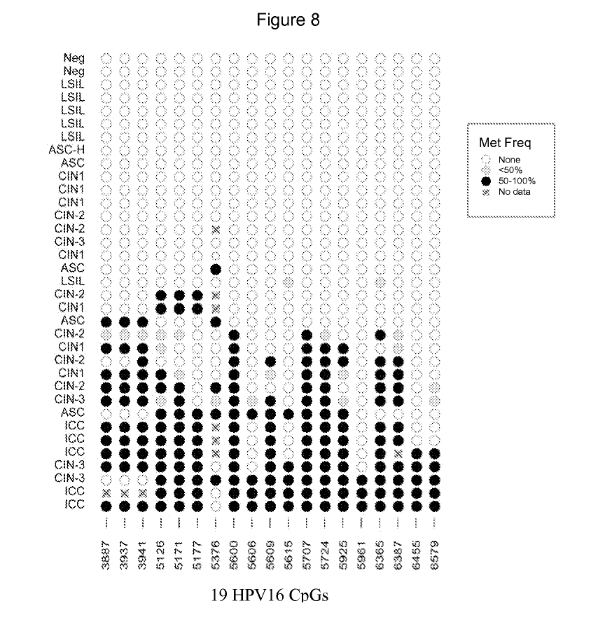

Natural history studies of CIN show that the risk of malignant progression is greatest for CIN-3 and least for CIN-1. Thus, the frequency of CpG methylation at the 19 CpGs correlates with the risk of malignant progression as assessed by histology. HPV16 DNA methylation analysis however measures biologic attributes other than conventional pathology because individual lesions with the same histologic diagnosis can have different patterns of HPV16 DNA methylation as shown in FIG. 8.

Mouse models of conditional transgene expression demonstrate that microscopic changes in cellular morphology (abnormal cytology/histology) are detectable only weeks to months after the transgene is transcriptionally activated or silenced. Cellular changes in transcription are immediately accompanied by corresponding changes in DNA methylation. It therefore follows that certain DNA methylation differences between normal and neoplastic cells, such as HPV-infected cervical cells, will be prognostic of the clinical outcome. Our data indicate that differences in the frequency and pattern of DNA methylation at the 19 HPV16 CpGs correlate with the risk of malignant progression and the likelihood of spontaneous regression.

x. Triage to Colposcopy

Four cytology specimens were unsatisfactory for cytologic evaluation (Unsat) and five were equivocal (ASCUS) (Table 14). In contrast, none of the HPV16 DNA methylation assays were either unsatisfactory or equivocal.

If the 22 cases with biopsy-confirmed diagnoses (Table 14) had been triaged on the basis of HPV16 DNA-positivity plus abnormal cytology, 21/22 cases (95.5%) would be referred for colposcopy (under existing protocols). In contrast, only 13/22 cases (59.0%) would be referred on the basis of HPV16 DNA methylation analysis, representing a highly significant reduction in unnecessary colposcopy referrals (P=0.0002, Fisher's exact test). At the same time, HPV DNA methylation analysis would have detected 100% of the cervical cancers, 75% of the CIN-3 lesions and 50% of the CIN-2 lesions. The undetected CIN-2/3 lesions may well have been at minimal risk of short-term progression to invasive cancer. Thus, a 12-month delay in therapeutic intervention, for example, would enable many of the lesions to spontaneously regress, thereby providing the patient with strong immunologic memory and almost certainly superior protection against recurrent CIN. Up to 20% to 25% of CIN lesions recur following therapeutic intervention, sometimes as a higher grade than the treated lesion.

In practice, HPV DNA methylation analysis is expected to have a greater impact on the number of unnecessary colposcopy referrals, because cervical cancer is a rare diagnosis. For example, if the five invasive cervical cancers were removed from the 22 cases, 16/17 cases (94%) would be referred on the basis of a combination of HPV16 DNA-positivity plus abnormal cytology vs. only 8/17 (47%) on the basis of HPV16 DNA methylation, representing a 47% reduction.

TABLE 14
Colposcopy Referral: Comparison of HPV16 DNA Methylation
Analysis vs. the Combination of HPV16 DNA+ plus Cytology

| | | | HPV16 | | |
|---|---|---|---|---|---|
| Case | Cytology | Colposcopy ^{a} | Met (%) ^{b} | Colposcopy | Diagnosis |
| 1 | Neg | ◯ | 0 | ◯ | CIN-1 |
| 2 | LSIL | | 0 | ◯ | CIN-1 |
| 3 | LSIL | | 0 | ◯ | CIN-1 |
| 4 | LSIL-H | | 63 ± 11 | | CIN-1 |
| 5 | ASC-H | | 5 ± 5 | ◯ | CIN-1 |
| 6 | ASCUS | | 47 ± 12 | | CIN-1 |
| 7 | ASCUS | | 53 ± 12 | | CIN-2 |
| 8 | ASCUS | | 63 ± 11 | | CIN-2 |
| 9 | ASCUS | | 79 ± 10 | | CIN-3 |
| 10 | HSIL | | 0 | ◯ | CIN-2 |
| 11 | HSIL | | 17 ± 9 | ◯ | CIN-2 |
| 12 | HSIL | | 42 ± 12 | | CIN-2 |
| 13 | HSIL | | 84 ± 9 | | CIN-3 |
| 14 | HSIL | | 94 ± 6 | | Cancer |
| 15 | Cancer | | 17 ± 9 | ◯ | CIN-1 |
| 16 | Cancer | | 72 ± 11 | | Cancer |
| 17 | Cancer | | 84 ± 9 | | CIN-3 |
| 18 | Cancer | | 95 ± 5 | | Cancer |
| 19 | Unsat ^{c} | | 0 | ◯ | CIN-2 |
| 20 | Unsat | | 0 | ◯ | CIN-3 |
| 21 | Unsat | | 72 ± 11 | | Cancer |
| 22 | Unsat | | 82 ± 10 | | Cancer |

^{a} Colposcopy: ◯ no referral, immediate referral unless <21 years.
^{b} Methylation at 19 CpGs (mean percent ± SEM).
^{c} Unsatisfactory for cytologic evaluation.

```
Human papillomavirus type 16 isolate 16W12E, complete genome
(Genbank AF125673.1; SEQ ID NO: 1):
      1 actacaataa ttcatgtata aaactaaggg cgtaaccgaa atcggttgaa ccgaaaccgg
    61 ttagtataaa agcagacatt ttatgcacca aaagagaact gcaatgtttc aggacccaca
  121 ggagcgaccc agaaagttac cacagttatg cacagagctg caaacaacta tacatgatat
  181 aatattagaa tgtgtgtact gcaagcaaca gttactgcga cgtgaggtat atgactttgc
  241 ttttcgggat ttatgtatag tatatagaga tgggaatcca tatgctgtat gtgataaatg
  301 tttaaagttt tattctaaaa ttagtgagta tagacattat tgttatagtg tgtatggaac
  361 aacattagaa cagcaataca acaaaccgtt gtgtgatttg ttaattaggt gtattaactg
  421 tcaaaagcca ctgtgtcctg aagaaaagca aagacatctg gacaaaaagc aaagattcca
  481 taatataagg ggtcggtgga ccggtcgatg tatgtcttgt tgcagatcat caagaacacg
  541 tagagaaacc cagctgtaat catgcatgga gatacaccta cattgcatga atatatgtta
  601 gatttgcaac cagagacaac tgatctctac tgttatgagc aattaaatga cagctcagag
  661 gaggaggatg aaatagatgg tccagctgga caagcagaac cggacagagc ccattacaat
  721 attgtaacct tttgttgcaa gtgtgactct acgcttcggt tgtgcgtaca aagcacacac
  781 gtagacattc gtactttgga agacctgtta atgggcacac taggaattgt gtgccccatc
  841 tgttctcaga aaccataatc taccatggct gatcctgcag gtaccaatgg ggaagagggt
  901 acgggatgta atggatggtt ttatgtagag gctgtagtgg aaaaaaaaac aggggatgct
  961 atatcagatg acgagaacga aaatgacagt gatacaggtg aagatttggt agattttata
1021 gtaaatgata atgattattt aacacaggca gaaacagaga cagcacatgc gttgtttact
1081 gcacaggaag caaaacaaca tagagatgca gtacaggttc taaaacgaaa gtatttgggt
1141 agtccactta gtgatattag tggatgtgta gacaataata ttagtcctag attaaaagct
1201 atatgtatag aaaaacaaag tagagctgca aaaaggagat tatttgaaag cgaagacagc
1261 gggtatggca atactgaagt ggaaactcag cagatgttac aggtagaagg gcgccatgag
1321 actgaaacac catgtagtca gtatagtggt ggaagtgggg gtggttgcag tcagtacagt
1381 agtggaagtg ggggagaggg tgttagtgaa agacacacta tatgccaaac accacttaca
1441 aatattttaa atgtactaaa aactagtaat gcaaaggcag caatgttagc aaaatttaaa
1501 gagttatacg gggtgagttt ttcagaatta gtaagaccat ttaaaagtaa taaatcaacg
1561 tgttgcgatt ggtgtattgc tgcatttgga cttacaccca gtatagctga cagtataaaa
1621 acactattac aacaatattg tttatattta cacattcaaa gtttagcatg ttcatgggga
1681 atggttgtgt tactattagt aagatataaa tgtggaaaaa atagagaaac aattgaaaaa
1741 ttgctgtcta aactattatg tgtgtctcca atgtgtatga tgatagagcc tccaaaattg
1801 cgtagtacag cagcagcatt atattggtat aaaacaggta tatcaaatat tagtgaagtg
1861 tatggagaca cgccagaatg gatacaaaga caaacagtat tacaacatag ttttaatgat
1921 tgtacatttg aattatcaca gatggtacaa tgggcctacg ataatgacat agtagacgat
1981 agtgaaattg catataaata tgcacaattg gcagacacta atagtaatgc aagtgccttt
2041 ctaaaaagta attcacaggc aaaaattgta aaggattgtg caacaatgtg tagacattat
2101 aaacgagcag aaaaaaaaca aatgagtatg agtcaatgga taaaatatag atgtgatagg
2161 gtagatgatg gaggtgattg gaagcaaatt gttatgtttt taaggtatca aggtgtagag
2221 tttatgtcat ttttaactgc attaaaaaga tttttgcaag gcatacctaa aaaaaattgc
2281 atattactat atggtgcagc taacacaggt aaatcattat ttggtatgag tttaatgaaa
2341 tttctgcaag ggtctgtaat atgttttgta aattctaaaa gccatttttg gttacaacca
2401 ttagcagatg ccaaaatagg tatgttagat gatgctacag tgccctgttg gaactacata
2461 gatgacaatt taagaaatgc attggatgga aatttagttt ctatggatgt aaagcataga
2521 ccattggtac aactaaaatg ccctccatta ttaattacat ctaacattaa tgctggtaca
2581 gattctaggt ggccttattt acataataga ttggtggtgt ttacatttcc taatgagttt
2641 ccatttgacg aaaacggaaa tccagtgtat gagcttaatg ataagaactg gaaatccttt
2701 ttctcaagga cgtggtccag attaagtttg cacgaggacg aggacaagga aaacgatgga
2761 gactctttgc caacgtttaa atgtgtgtca ggacaaaata ctaacacatt atgaaaatga
2821 tagtacagac ctacgtgacc atatagacta ttggaaacac atgcgcctag aatgtgctat
2881 ttattacaag gccagagaaa tgggatttaa acatattaac caccaggtgg tgccaacact
2941 ggctgtatca aagaataaag cattacaagc aattgaactg caactaacgt tagaaacaat
3001 atataactca caatatagta atgaaaagtg gacattacaa gacgttagcc ttgaagtgta
3061 tttaactgca ccaacaggat gtataaaaaa acatggatat acagtggaag tgcagtttga
3121 tggagacata tgcaatacaa tgcattatac aaactggaca catatatata tttgtgaaga
3181 agcatcagta actgtggtag agggtcaagt tgactattat ggtttatatt atgttcatga
3241 aggaatacga acatattttg tgcagtttaa agatgatgca gaaaaatata gtaaaaataa
3301 agtatgggaa gttcatgcgg gtggtcaggt aatattatgt cctacatctg tgtttagcag
3361 caacgaagta tcctctcctg aaattattag gcagcacttg gccaaccact ccgccgcgac
3421 ccataccaaa gccgtcgcct tgggcaccga agaaacacag acgactatcc agcgaccaag
3481 atcagagcca gacaccggaa acccctgcca caccactaag ttgttgcaca gagactcagt
3541 ggacagtgct ccaatcctca ctgcatttaa cagctcacac aaaggacgga ttaactgtaa
3601 tagtaacact acacccatag tacatttaaa aggtgatgct aatactttaa aatgtttaag
3661 atatagattt aaaaagcatt gtacattgta tactgcagtg tcgtctacat ggcattggac
3721 aggacataat gtaaaacata aaagtgcaat tgttacactt acatatgata gtgaatggca
3781 acgtgaccaa tttttgtctc aagttaaaat accaaaaact attacagtgt ctactggatt
3841 tatgtctata tgacaaatct tgatactgca tccacaacat tactggcgtg ctttttgctt
3901 tgcttttgtg tgcttttgtg tgtctgccta ttaatacgtc cgctgctttt gtctgtgtct
3961 acatacacat cattaatact attggtatta ctattgtgga taacagcagc ctctgcgttt
4021 aggtgtttta ttgtatatat tgtatttgtt tatataccat tatttttaat acatacacat
4081 gcacgctttt taattacata atgtatatgt acaaaatgta attgttacat ataattgttg
4141 tataccataa cttactattt tttctttttt attttcatat atattttttt tttgtttgtt
4201 tgtttgtttt ttaataaact gttatcactt aacaatgcga cacaaacgtt ctgcaaaacg
4261 cacaaaacgt gcatcggcta cccaacttta taaaacatgc aaacaggcag gtacatgtcc
4321 acctgacatt atacctaagg ttgaaggcaa aactattgct gatcaaatat tacaatatgg
4381 aagtatgggt gtattttttg gtgggttagg aattggaaca gggtcgggta caggcggacg
4441 cactgggtat attccattgg gaacaaggcc tcccacagct acagatacac ttgctcctgt
4501 aagaccccct ttaacagtag atcctgtggg cccttccgat ccttctatag tttctttagt
4561 ggaagaaact agttttattg atgctggtgc accaacatct gtaccttcca ttcccccaga
4621 tgtatcagga tttagtatta ctacttcaac tgataccaca cctgctatat tagatattaa
4681 taatactgtt actactgtta ctacacataa taatcccact ttcactgacc catctgtatt
4741 gcagcctcca acacctgcag aaactggagg gcattttaca ctttcatcat ccactattag
4801 tacacataat tatgaagaaa ttcctatgga tacatttatt gttagcacaa accctaacac
4861 agtaactagt agcacaccca taccagggtc tcgcccagtg gcacgcctag gattatatag
4921 tcgcacaaca caacaagtta aagttgtaga ccctgctttt ataaccactc ccactaaact
4981 tattacatat gataatcctg catatgaagg tatagatgtg gataatacat tatatttttc
5041 tagtaatgat aatagtatta atatagctcc agatcctgac tttttggata tagttgcttt
5101 acataggcca gcattaacct ctaggcgtac tggcattagg tacagtagaa ttggtaataa
5161 acaaacacta cgtactcgta gtggaaaatc tataggtgct aaggtacatt attattatga
5221 ttttagtact attgattctg cagaagaaat agaattacaa actataacac cttctacata
5281 tactaccact tcacatgcag ccttacctac ttctattaat aatggattat atgatattta
5341 tgcagatgac tttattacag atacttctac aaccccggta ccatctgtac cctctacatc
5401 tttatcaggt tatattcctg caaatacaac aattcctttt ggtggtgcat acaatattcc
5461 tttagtatca ggtcctgata tacccattaa tataactgac caagctcctt cattaattcc
5521 tatagttcca gggtctccac aatatacaat tattgctgat gcaggtgact tttatttaca
5581 tcctagttat tacatgttac gaaaacgacg taaacgttta ccatattttt tttcagatgt
5641 ctctttggct gcctagtgag gccactgtct acttgcctcc tgtcccagta tctaaggttg
5701 taagcacgga tgaatatgtt gcacgcacaa acatatatta tcatgcagga acatccagac
5761 tacttgcagt tggacatccc tattttccta ttaaaaaacc taacaataac aaaatattag
5821 ttcctaaagt atcaggatta caatacaggg tatttagaat acatttacct gaccccaata
5881 agtttggttt tcctgacacc tcattttata atccagatac acagcggctg gtttgggcct
5941 gtgtaggtgt tgaggtaggc cgtggtcagc cattaggtgt gggcattagt ggccatcctt
6001 tattaaataa attggatgac acagaaaatg ctagtgctta tgcagcaaat gcaggtgtgg
6061 ataatagaga atgtatatct atggattaca aacaaacaca attgtgttta attggttgca
6121 aaccacctat aggggaacac tggggcaaag gatccccatg taccaatgtt gcagtaaatc
6181 caggtgattg tccaccatta gagttaataa acacagttat tcaggatggt gatatggttg
6241 atactggctt tggtgctatg gactttacta cattacaggc taacaaaagt gaagttccac
6301 tggatatttg tacatctatt tgcaaatatc cagattatat taaaatggtg tcagaaccat
6361 atggcgacag cttatttttt tatttacgaa gggaacaaat gtttgttaga catttattta
6421 atagggctgg tgctgttggt gaaaatgtac cagacgattt atacattaaa ggctctgggt
6481 ctactgcaaa tttagccagt tcaaattatt ttcctacacc tagtggttct atggttacct
6541 ctgatgccca aatattcaat aaaccttatt ggttacaacg agcacagggc cacaataatg
6601 gcatttgttg gggtaaccaa ctatttgtta ctgttgttga tactacacgc agtacaaata
6661 tgtcattatg tgctgccata tctacttcag aaactacata taaaaatact aactttaagg
6721 agtacctacg acatggggag gaatatgatt tacagtttat ttttcaactg tgcaaaataa
6781 ccttaactgc agacgttatg acatacatac attctatgaa ttccactatt ttggaggact
6841 ggaattttgg tctacaacct cccccaggag gcacactaga agatacttat aggtttgtaa
6901 catcccaggc aattgcttgt caaaaacata cacctccagc acctaaagaa gatcccctta
6961 aaaaatacac tttttgggaa gtaaatttaa aggaaaagtt ttctgcagac ctagatcagt
7021 ttcctttagg acgcaaattt ttactacaag caggattgaa ggccaaacca aaatttacat
7081 taggaaaacg aaaagctaca cccaccacct catctacctc tacaactgct aaacgcaaaa
7141 aacgtaagct gtaagtattg tatgtatgtt gaattagtgt tgtttgttgt ttatatgttt
7201 gtatgtgctt gtatgtgctt gtaaatatta agttgtatgt gtgtttgtat gtatggtata
7261 ataaacacgt gtgtatgtgt ttttaaatgc ttgtgtaact attgtgtcat gcaacataaa
7321 taaacttatt gtttcaacac ctactaattg tgttgtggtt attcattgta tataaactat
7381 atttgctaca tcctgttttt gttttatata tactaaattt tgtagcgcca gcggccattt
7441 tgtagcttca accgaattcg gttgcatgct ttttggcaca aaatgtgttt ttttaaatag
7501 ttctatgtca gcaactatag tttaaacttg tacgtttcct gcttgccatg cgtgccaaat
7561 ccctgttttc ctgacctgca ctgcttgcca accattccat tgttttttac actgcactat
7621 gtgcaactac tgaatcacta tgtacattgt gtcatataaa ataaatcact atgcgccaac
7681 gccttacata ccgctgttag gcacatattt ttggcttgtt ttaactaacc taattgcata
7741 tttggcataa ggtttaaact tctaaggcca actaaatgtc accctagttc atacatgaac
7801 tgtgtaaagg ttagtcatac attgttcatt tgtaaaactg cacatgggtg tgtgcaaacc
7861 gttttgggtt acacatttac aagcaactta tataataata ctaa
```

### REFERENCES

Alazawi W, Pett M, Arch B, Scott L, Freeman T, Stanley Mass., Coleman N. Changes in cervical keratinocyte gene expression associated with integration of human papillomavirus 16. Cancer Research 2002; 62(23):6959-65. [PubMed: 12460913]
American Cancer Society. Cancer Facts & FIGS. 2007. ACS; 2007.
Badal S, Badal V, Calleja-Macias I E, Kalantari M, Chuang L S H, Li B F L, Bernard H-U. The human papillomavirus-18 genome is efficiently targeted by cellular DNA methylation. Virology 2004; 324(2):483-92. [PubMed: 15207633]
Badal V, Chuang LSH, Tan E H-H, Badal S, Villa L L, Wheeler C M, Li B F L, Bernard H-U. CpG methylation of human papillomavirus type 16 DNA in cervical cancer cell lines and in clinical specimens: genomic hypomethylation correlates with carcinogenic progression. Journal of Virology 2003; 77(11):6227-34. [PubMed: 12743279]
Balderas-Loaeza A, Anaya-Saavedra G, Ramirez-Amador V A, Guido-Jimenez M C, Kalantari M, Calleja-Macias I E, Bernard H-U, Garcia-Carranca A. Human papillomavirus-16 DNA methylation patterns support a causal association of the virus with oral squamous cell carcinomas. International Journal of Cancer 2007; 120(10):2165-9.
Ballestar E, Esteller M. Epigenetic gene regulation in cancer. Advances in Genetics 2008; 61:247-67. [PubMed: 18282509]
Bernstein B E, Meissner A, Lander E S. The mammalian epigenome. Cell 2007; 128(4):669-81. [PubMed: 17320505]
Brandsma J L, Sun Y, Lizardi P M, Tuck D P, Zelterman D, et al. (2009) Distinct Human Papillomavirus Type 16 Methylomes in Cervical Cells at Different Stages of Premalignancy. Virology 389:100-107.
DeFilippis R, Goodwin E, Wu L, DiMaio D. Endogenous human papillomavirus E6 and E7 proteins differentially regulate proliferation, senescence, and apoptosis in HeLa cervical carcinoma cells. Journal of Virology 2003; 77(2):1551-63. [PubMed: 12502868]
DiMaio D, Miller G. Thirty years into the genomics era: tumor viruses led the way. Yale Journal of Biology & Medicine 2006; 79(3-4):179-85. [PubMed: 17940630]
Dunne E F, Unger E R, Sternberg M, McQuillan G, Swan D C, et al. (2007) Prevalence of HPV infection among females in the United States. JAMA 297:813-819. [5] [PubMed: 17327523]
Esteller M. CpG island hypermethylation and tumor suppressor genes: a booming present, a brighter future. Oncogene 2002; 21(35):5427-40. [PubMed: 12154405]
Esteller, M. (2007). Cancer epigenomics: DNA methylomes and histone-modification maps. Nature Reviews Genetics 8(4):286-98.
Feng, Q., Hawes, S. E., Stern, J. E., Wiens, L., Lu, H., Dong, Z. M., Jordan, C. D., Kiviat, N. B., and Vesselle, H. (2008). DNA methylation in tumor and matched normal tissues from non-small cell lung cancer patients. Cancer Epidemiology, Biomarkers & Prevention 17(3):645-54.
Flores E R, Allen-Hoffmann B L, Lee D, Sattler C A, Lambert P F. Establishment of the human papillomavirus type 16 (HPV-16) life cycle in an immortalized human foreskin keratinocyte cell line. Virology 1999; 262(2):344-54. [PubMed: 10502513]
Frühwald M C (2003) DNA Methylation Patterns in Cancer: Novel Prognostic Indicators? American Journal of PharmacoGenomics 3:245-26Q.
Goldhaber-Fiebert J D, Stout N K, Salomon J A, Kuntz K M, Goldie S J. Cost-effectiveness of cervical cancer screening with human papillomavirus DNA testing and HPV-16,18 vaccination. Journal of the National Cancer Institute 2008; 100(5):308-20. [PubMed: 18314477]
Gopalakrishnan, S., Van Emburgh, B. O., and Robertson, K. D. (2008). DNA methylation in development and human disease. Mutation Research 647(1-2):30-8.
Henk H J, lnsinga R P, Singhal P K, Darkow T (2010) Incidence and Costs of Cervical Intraepithelial Neoplasia in a US Commercially Insured Population. Journal of Lower Genital Tract Disease 14:29-36.
Insinga R P, Glass A G, Rush B B (2004) The health care costs of cervical human papillomavirus:related disease. American Journal of Obstetrics & Gynecology 191:114-120.
Insinga, R. P., Dasbach, E. J., and Elbasha, E. H. (2005). Assessing the annual economic burden of preventing and treating anogenital human papillomavirus-related disease in the US: analytic framework and review of the literature. Pharmacoeconomics 23(11), 1107-22.
Kalantari M, Calleja-Macias I E, Tewari D, Hagmar B, Lie K, Barrera-Saldana H A, Wiley D J, Bernard H-U. Conserved methylation patterns of human papillomavirus type 16 DNA in asymptomatic infection and cervical neoplasia. Journal of Virology 2004; 78(23):12762-72. [PubMed: 15542628]
Kalantari M, Lee D, Calleja-Macias I E, Lambert P F, Bernard H-U. Effects of cellular differentiation, chromosomal integration and 5-aza-2′-deoxycytidine treatment on human papillomavirus-16 DNA methylation in cultured cell lines. Virology 2008a; 374(2):292-303. [PubMed: 18242658]
Kalantari M, Villa L L, Calleja-Macias I E, Bernard H-U. Human papillomavirus-16 and -18 in penile carcinomas: DNA methylation, chromosomal recombination and genomic variation. International Journal of Cancer 2008b; 123(8):1832-40.
Kim K, Garner-Hamrick P A, Fisher C, Lee D, Lambert P F. Methylation patterns of papillomavirus DNA, its influence on E2 function, and implications in viral infection. Journal of Virology 2003; 77(23):12450-9. [PubMed: 14610169]
Kiviat N B, Hawes S E, Feng Q. Screening for cervical cancer in the era of the HPV vaccine—the urgent need for both new screening guidelines and new biomarkers. Journal of the National Cancer Institute 2008; 100(5):290-1. [PubMed: 18314471]
Laird P W (2003) The power and the promise of DNA methylation markers. Nat Rev Cancer 3: 253-266.
Li, L-C. MethPrimer—Design Primers for Methylation PCRs.
Paradisi A, Pasquariello N, Barcaroli D, Maccarrone M. Anandamide regulates keratinocyte differentiation by inducing DNA methylation in a CB1 receptor-dependent manner. Journal of Biological Chemistry 2008; 283(10):6005-12. [PubMed: 18165231]
Schiffman M, Herrero R, Desalle R, Hildesheim A, Wacholder S, Rodriguez A C, Bratti M C, Sherman M E, Morales J, Guillen D, Alfaro M, Hutchinson M, Wright T C, Solomon D, Chen Z, Schussler J, Castle P E, Burk R D. The carcinogenicity of human papillomavirus types reflects viral evolution. Virology 2005; 337(1):76-84. [PubMed: 15914222]
Schorge J O, Knowles L M, Lea J S (2004) Adenocarcinoma of the cervix. Current Treatment Options in Oncology 5:119-127.
Spitzer M. In vitro conventional cytology historical strengths and current limitations. Obstetrics & Gynecology Clinics of North America 2002; 29(4):673-83. [PubMed: 12509091]
Suzuki R, Shimodaira H. Pvclust: an R package for assessing the uncertainty in hierarchical clustering. Bioinformatics 2006; 22(12):1540-2. [PubMed: 16595560]
Team, R D C. R: A Language and Environment for Statistical Computing. R Foundation for Statistical Computing; Vienna, Austria: 2007.
Trottier H, Mahmud S, Costa M C, Sobrinho J P, Duarte-Franco E, Rohan T E, Ferenczy A, Villa L L, Franco E L. Human papillomavirus infections with multiple types and risk of cervical neoplasia. Cancer Epidemiology, Biomarkers & Prevention 2006; 15(7):1274-80.
Turan T, Kalantari M, Calleja-Macias I E, Cubie H A, Cuschieri K, Villa L L, Skomedal H, Barrera-Saldana H A, Bernard H-U. Methylation of the human papillomavirus-18 L1 gene: a biomarker of neoplastic progression? Virology 2006; 349(1):175-83. [PubMed: 16472835]
Turan T, Kalantari M, Cuschieri K, Cubie H A, Skomedal H, Bernard H-U. High-throughput detection of human papillomavirus-18 L1 gene methylation, a candidate biomarker for the progression of cervical neoplasia. Virology 2007; 361(1):185-93. [PubMed: 17175003]
Vinokurova S, Wentzensen N, Kraus I, Klaes R, Driesch C, Melsheimer P, Kisseljov F, Durst M, Schneider A, von Knebel Doeberitz M. Type-dependent integration frequency of human papillomavirus genomes in cervical lesions. Cancer Research 2008; 68(1):307-13. [PubMed: 18172324]
Wade N (2010) DNA Test May Speed Colon Cancer Diagnosis. The New York Times. New York: The New York Times.
Wettstein F O, Stevens J G. Shope papilloma virus DNA is extensively methylated in non-virusproducing neoplasms. Virology 1983; 126(2):493-504. [PubMed: 6305000]
Wiley D J, Huh J, Rao J Y, Chang C, Goetz M, Poulter M, Masongsong E, Chang C I, Bernard H-U. Methylation of human papillomavirus genomes in cells of anal epithelia of HIV-infected men. Journal of Acquired Immune Deficiency Syndromes: JAIDS 2005; 39(2):143-51.
Zheng Z-M, Baker C C. Papillomavirus genome structure, expression, and post-transcriptional regulation. Frontiers in Bioscience 2006; 11:2286-302. [PubMed: 16720315]
zur Hausen H. Viruses in human cancers. Science 1991; 254(5035):1167-73. [PubMed: 1659743]

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a CpG dinucleotide” includes a plurality of such CpG dinucleotides, reference to “the CpG dinucleotide” is a reference to one or more CpG dinucleotides and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

Those skilled in the art will recognize, or be able to ascertain using no more than routine experimentation, many equivalents to the specific embodiments of the method and compositions described herein. Such equivalents are intended to be encompassed by the following claims.

1. A method comprising
determining the methylation state of a set of CpG dinucleotides in HPV16 sequences in a sample of a subject, wherein the set comprises a set of 3 or more of the CpG dinucleotides that correspond to positions 3887, 3937, 3941, 4016, 4084, 4238, 4247, 4259, 4268, 4275, 4425, 4435, 4439, 4537, 4892, 4904, 4922, 5126, 5171, 5177, 5376, 5600, 5606, 5609, 5615, 5707, 5724, 5925, 5961, 6365, 6387, 6455, 6579, 6648, 6729, 6794, 7032, and 7089 of human papillomavirus 16 (HPV16),
identifying the cancer potential state of the subject based on the methylation state of the set of 3 or more CpG dinucleotides.

2. The method of claim 1, wherein a methylation site percentage for the set of 3 or more CpG dinucleotides of over 37.0% indicates that the subject should be tested further.

3. The method of claim 1, wherein a methylation site percentage for the set of 3 or more CpG dinucleotides of up to 37.0% indicates that the subject need not be tested further for at least 6 months.

4. The method of claim 1, wherein the set of 3 or more CpG dinucleotides comprises the CpG dinucleotide corresponding to position 5177 of HPV16.

5. The method of claim 4, wherein the set of 3 or more CpG dinucleotides further comprises the CpG dinucleotide corresponding to position 6455 of HPV16.

6. The method of claim 5, wherein the set of 3 or more CpG dinucleotides further comprises the CpG dinucleotides corresponding to positions 3887, 3937, 3941, 5126, 5171, 5600, 5609, 5707, 5724, 5925, and 6365 of HPV16.

7. The method of claim 5, wherein the set of 3 or more CpG dinucleotides further comprises the CpG dinucleotides corresponding to positions 5615 and 5961 of HPV16.

8. The method of claim 7, wherein the set of 3 or more CpG dinucleotides further comprises the CpG dinucleotides corresponding to positions 5126, 5171, 5600, 5609, 5707, 5724, 5925, and 6365 of HPV16.

9. The method of claim 8, wherein the set of 3 or more CpG dinucleotides further comprises the CpG dinucleotides corresponding to positions 3887, 3937, and 3941 of HPV16.

10. The method of claim 9, wherein the set of 3 or more CpG dinucleotides further comprises the CpG dinucleotides corresponding to positions 5376, 5606, 6387, and 6579 of HPV16.

11. The method of claim 4, wherein the set of 3 or more CpG dinucleotides further comprises the CpG dinucleotides corresponding to positions 5126, 5171, 5600, 5609, 5707, 5724, 5925, and 6365 of HPV16.

12. The method of claim 1, wherein the set 3 or more CpG dinucleotides comprises the CpG dinucleotides that correspond to positions 3887, 3937, 3941, 4439, 5126, 5600, 5707, 5724, and 6387 of HPV16.

13. The method of claim 12, wherein the set of 3 or more CpG dinucleotides further comprises the CpG dinucleotides corresponding to positions 4435 and 6729 of HPV16.

14. The method of claim 1, wherein a methylation site percentage for the set of 3 or more CpG dinucleotides of below 20.0% indicates a low cancer potential state,
wherein a methylation site percentage for the set of 3 or more CpG dinucleotides from 20.0% to 50.0% indicates a moderate cancer potential state, and
wherein a methylation site percentage for the set of 3 or more CpG dinucleotides of over 50.0% indicates a high cancer potential state.

15. The method of claim 1, wherein identification of the cancer potential state of the subject is further based on both cytologic screening and on the methylation state of the set of CpG dinucleotides.

16. The method of claim 15, wherein a cytology assessment of low-grade squamous intraepithelial lesion (LSIL) and a methylation site percentage for the set of 3 or more CpG dinucleotides of below 20.0% indicates a low cancer potential state,
wherein a methylation site percentage for the set of 3 or more CpG dinucleotides from 20.0% to 50.0% indicates a moderate cancer potential state, and
wherein a methylation site percentage for the set of 3 or more CpG dinucleotides of over 50.0% indicates a high cancer potential state.

17. The method of claim 15, wherein a cytology assessment of atypical squamous cells (ASC) and a methylation site percentage for the set of 3 or more CpG dinucleotides of below 15.0% indicates a low cancer potential state,
wherein a cytology assessment of ASC and a methylation site percentage for the set of 3 or more CpG dinucleotides from 15.0% to 35.0% indicates a moderate cancer potential state, and
wherein a cytology assessment of ASC and a methylation site percentage for the set of 3 or more CpG dinucleotides of over 35.0% indicates a high cancer potential state.

18. The method of claim 15, wherein a cytology assessment of high-grade squamous intraepithelial lesion (HSIL) and a methylation site percentage for the set of 3 or more CpG dinucleotides of below 10.0% indicates a low cancer potential state,
wherein a cytology assessment of HSIL and a methylation site percentage for the set of 3 or more CpG dinucleotides from 10.0% to 35.0% indicates a moderate cancer potential state, and
wherein a cytology assessment of HSIL and a methylation site percentage for the set of 3 or more CpG dinucleotides of over 35.0% indicates a high cancer potential state.

19. The method of claim 15, wherein a methylation site percentage for the set of 3 or more CpG dinucleotides of over 37.0% indicates that the subject should be tested further.

20. The method of claim 19, wherein a methylation site percentage for the set of 3 or more CpG dinucleotides of over 37.0% indicates that the subject should be referred for colposcopy.

21. The method of claim 15, wherein a methylation site percentage for the set of 3 or more CpG dinucleotides of up to 37.0% indicates that the subject need not be tested further for at least 6 months.

22. The method of claim 1, wherein identification of the cancer potential state of the subject is further based on both histologic screening and on the methylation state of the set of CpG dinucleotides.

23. The method of claim 22, wherein a histology assessment of HPV-associated intraepithelial neoplasia grade 1 and a methylation site percentage for the set of 3 or more CpG dinucleotides of below 20.0% indicates a low cancer potential state,
wherein a methylation site percentage for the set of 3 or more CpG dinucleotides from 20.0% to 50.0% indicates a moderate cancer potential state, and
wherein a methylation site percentage for the set of 3 or more CpG dinucleotides of over 50.0% indicates a high cancer potential state.

24. The method of claim 22, wherein a histology assessment of HPV-associated intraepithelial neoplasia grade 2 and a methylation site percentage for the set of 3 or more CpG dinucleotides of below 15.0% indicates a low cancer potential state,
wherein a histology assessment of HPV-associated intraepithelial neoplasia grade 2 and a methylation site percentage for the set of 3 or more CpG dinucleotides from 15.0% to 50.0% indicates a moderate cancer potential state, and
wherein a histology assessment of HPV-associated intraepithelial neoplasia grade 2 and a methylation site percentage for the set of 3 or more CpG dinucleotides over 35.0% indicates a high cancer potential state.

25. The method of claim 22, wherein a histology assessment of HPV-associated intraepithelial neoplasia grade 3 and a methylation site percentage for the set of 3 or more CpG dinucleotides of below 35.0% indicates a moderate cancer potential state, and
wherein a histology assessment of HPV-associated intraepithelial neoplasia grade 3 and a methylation site percentage for the set of 3 or more CpG dinucleotides over 35.0% indicates a high cancer potential state.

26. The method of claim 1, wherein identification of the cancer potential state of the subject is further based on cytologic screening, on histologic screening, and on the methylation state of the set of CpG dinucleotides.

27. The method of claim 26, wherein a cytology assessment of LSIL, a histology assessment of HPV-associated intraepithelial neoplasia grade 1, and a methylation site percentage for the set of 3 or more CpG dinucleotides of below 20.0% indicates a low cancer potential state,
wherein a methylation site percentage for the set of 3 or more CpG dinucleotides from 20.0% to 50.0% indicates a moderate cancer potential state, and
wherein a methylation site percentage for the set of 3 or more CpG dinucleotides of over 50.0% indicates a high cancer potential state.

28. The method of claim 26, wherein a cytology assessment of LSIL, a histology assessment of HPV-associated intraepithelial neoplasia grade 2, and a methylation site percentage for the set of 3 or more CpG dinucleotides of below 15.0% indicates a low cancer potential state,
wherein a cytology assessment of LSIL, a histology assessment of HPV-associated intraepithelial neoplasia grade 2, and a methylation site percentage for the set of 3 or more CpG dinucleotides from 15.0% to 50.0% indicates a moderate cancer potential state, and
wherein a cytology assessment of LSIL, a histology assessment of HPV-associated intraepithelial neoplasia grade 2, and a methylation site percentage for the set of 3 or more CpG dinucleotides of over 50.0% indicates a high cancer potential state.

29. The method of claim 26, wherein a cytology assessment of LSIL, a histology assessment of HPV-associated intraepithelial neoplasia grade 3, and a methylation site percentage for the set of 3 or more CpG dinucleotides of below 10.0% indicates a low cancer potential state,
wherein a cytology assessment of LSIL, a histology assessment of HPV-associated intraepithelial neoplasia grade 3, and a methylation site percentage for the set of 3 or more CpG dinucleotides from 10.0% to 35.0% indicates a moderate cancer potential state, and
wherein a cytology assessment of LSIL, a histology assessment of HPV-associated intraepithelial neoplasia grade 3, and a methylation site percentage for the set of 3 or more CpG dinucleotides of over 35.0% indicates a high cancer potential state.

30. The method of claim 26, wherein a cytology assessment of ASC, a histology assessment of HPV-associated intraepithelial neoplasia grade 1, and a methylation site percentage for the set of 3 or more CpG dinucleotides of below 15.0% indicates a low cancer potential state,
wherein a cytology assessment of ASC, a histology assessment of HPV-associated intraepithelial neoplasia grade 1, and a methylation site percentage for the set of 3 or more CpG dinucleotides from 15.0% to 50.0% indicates a moderate cancer potential state, and
wherein a cytology assessment of ASC, a histology assessment of HPV-associated intraepithelial neoplasia grade 1, and a methylation site percentage for the set of 3 or more CpG dinucleotides of over 50.0% indicates a high cancer potential state.

31. The method of claim 26, wherein a cytology assessment of ASC, a histology assessment of HPV-associated intraepithelial neoplasia grade 2, and a methylation site percentage for the set of 3 or more CpG dinucleotides of below 15.0% indicates a low cancer potential state,
wherein a cytology assessment of ASC, a histology assessment of HPV-associated intraepithelial neoplasia grade 2, and a methylation site percentage for the set of 3 or more CpG dinucleotides from 15.0% to 35.0% indicates a moderate cancer potential state, and
wherein a cytology assessment of ASC, a histology assessment of HPV-associated intraepithelial neoplasia grade 2, and a methylation site percentage for the set of 3 or more CpG dinucleotides of over 35.0% indicates a high cancer potential state.

32. The method of claim 26, wherein a cytology assessment of ASC, a histology assessment of HPV-associated intraepithelial neoplasia grade 3, and a methylation site percentage for the set of 3 or more CpG dinucleotides of below 35.0% indicates a moderate cancer potential state, and
wherein a cytology assessment of ASC, a histology assessment of HPV-associated intraepithelial neoplasia grade 3, and a methylation site percentage for the set of 3 or more CpG dinucleotides of over 35.0% indicates a high cancer potential state.

33. The method of claim 26, wherein a cytology assessment of HSIL, a histology assessment of HPV-associated intraepithelial neoplasia grade 1, and a methylation site percentage for the set of 3 or more CpG dinucleotides of below 15.0% indicates a low cancer potential state,
wherein a cytology assessment of HSIL, a histology assessment of HPV-associated intraepithelial neoplasia grade 1, and a methylation site percentage for the set of 3 or more CpG dinucleotides from 15.0% to 35.0% indicates a moderate cancer potential state, and
wherein a cytology assessment of HSIL, a histology assessment of HPV-associated intraepithelial neoplasia grade 1, and a methylation site percentage for the set of 3 or more CpG dinucleotides of over 35.0% indicates a high cancer potential state,

34. The method of claim 26, wherein a cytology assessment of HSIL, a histology assessment of HPV-associated intraepithelial neoplasia grade 2, and a methylation site percentage for the set of 3 or more CpG dinucleotides of below 10.0% indicates a low cancer potential state,
wherein a cytology assessment of HSIL, a histology assessment of HPV-associated intraepithelial neoplasia grade 2, and a methylation site percentage for the set of 3 or more CpG dinucleotides from 10.0% to 35.0% indicates a moderate cancer potential state, and
wherein a cytology assessment of HSIL, a histology assessment of HPV-associated intraepithelial neoplasia grade 2, and a methylation site percentage for the set of 3 or more CpG dinucleotides of over 35.0% indicates a high cancer potential state,

35. The method of claim 26, wherein a cytology assessment of HSIL, a histology assessment of HPV-associated intraepithelial neoplasia grade 3, and a methylation site percentage for the set of 3 or more CpG dinucleotides of below 20.0% indicates a moderate cancer potential state, and
wherein a cytology assessment of HSIL, a histology assessment of HPV-associated intraepithelial neoplasia grade 3, and a methylation site percentage for the set of 3 or more CpG dinucleotides of over 20.0% indicates a high cancer potential state.

36. The method of claim 1 further comprising performing a histologic screen of the subject for cervical cancer potential at an interval based on the identified cancer potential state.

37. The method of claim 1 further comprising creating a record of the identification of the cancer potential state of the subject.

38. The method of claim 1 further comprising testing the subject for cervical cancer potential at an interval based on the identified cancer potential state.

39. The method of claim 1 further comprising testing the subject for cervical cancer potential in a mode or manner based on the identified cancer potential state.

40. The method of claim 1 further comprising treating the subject at an interval based on the identified cancer potential state.

41. The method of claim 1 further comprising treating the subject in a mode or manner based on the identified cancer potential state.

42. A method comprising
determining the methylation state of a set of CpG dinucleotides in HPV16 sequences in a sample of a subject,
identifying the cancer potential state of the subject based on the pattern of methylation of the set of CpG dinucleotides,
wherein a pattern of methylation corresponding to methylation pattern A indicates a low cancer potential state, and
wherein a pattern of methylation corresponding to methylation pattern C indicates a high cancer potential state.

43. The method of claim 1, wherein the set of CpG dinucleotides for which the methylation state is determined comprises the 113 CpG dinucleotides of HPV16.
